(19) United States
(12) Patent Application Publication
(10) Pub. No.: US 2025/0230429 A1
(43) Pub. Date: Jul. 17, 2025

(54) Continuous Multiplexed Phage Genome Engineering Using a Retron Editing Template

(71) Applicant: The J. David Gladstone Institutes, a testamentary trust established under the Will of J. David Glads, San Francisco, CA (US)

(72) Inventors: Seth Shipman, San Francisco, CA (US); Chloe Fishman, San Francisco, CA (US); Santi Bhattarai-Kline, San Francisco, CA (US)

(21) Appl. No.: 18/853,847

(22) PCT Filed: Mar. 9, 2023
(86) PCT No.: PCT/US2023/064014

§ 371 Date: Oct. 3, 2024

Related U.S. Application Data
(60) Provisional application No. 63/328,387, filed on Apr. 7, 2022.

(51) Int. Cl. C12N 15/10; C12N 1/20; C12N 7/00; C12N 9/12; C12N 9/22; C12N 15/73; C12N 15/90

(52) U.S. Cl. CPC C12N 15/102; C12N 1/20; C12N 7/00; C12N 9/1276; C12N 9/22; C12N 15/73; C12N 15/902; C12N 2795/00022; C12N 2795/00051; C12Y 207/07049

(57) ABSTRACT

Systems and methods for editing bacteriophages are described herein.

### PRIORITY APPLICATION

This application is U.S. National Stage Filing under 35 U.S.C. 371 from International Patent Application Serial No. PCT/US2023/064014, filed Mar. 9, 2023, published on Oct. 12, 2023 as WO 2023/196725, which application claims the benefit of priority to U.S. Provisional Patent Application Ser. No. 63/328,387, filed Apr. 7, 2022, the content of which are incorporated herein by reference in their entirety.

### INCORPORATION BY REFERENCE OF SEQUENCE LISTING

A Sequence Listing is provided herewith as an xml file, “3730211US1.xml” created on Nov. 4, 2024, and having a size of 63,991 bytes. The content of the xml file is incorporated by reference herein in its entirety.

### BACKGROUND

Antibiotic resistance is increasing in all parts of the world. New resistance mechanisms are emerging and spreading globally, threatening our ability to treat common infectious diseases. A growing list of infections—such as pneumonia, tuberculosis, blood poisoning, gonorrhea, and foodborne diseases—are becoming harder, and sometimes almost impossible, to treat as antibiotics become less effective.

While there are some new antibiotics in development, few if any of them are expected to be effective against the most dangerous forms of antibiotic-resistant bacteria. Moreover, given the ease and frequency with which people now travel, antibiotic resistance is a global problem. New innovations and efforts to combat this problem are needed.

### SUMMARY

Described herein are compositions and methods for genetically modifying bacteriophage. Such compositions and methods can be used to generate bacteriophage antimicrobials. Bacteriophages can kill bacteria, but natural bacteriophages are limited in their host range and are subject to a myriad of bacterial anti-phage systems. However, the compositions and methods described herein can be used to engineer purpose-built bacteriophages that target the bacteria causing disease and that can escape the anti-phage defenses of the targeted bacteria. Hence, the compositions and methods can generate engineered bacteriophage for use as therapeutics to cure resistant bacterial infections.

The engineered phage antimicrobials can also be used industrially. For example, engineered phage can be used for clearing unwanted bacteria from industrial processes, instead of harsh chemicals. Such industrial uses can reduce the need for human intervention. In another example, the engineered phage can be used as delivery agents that serve as vectors to deliver cargo such as DNA, RNA, or protein to cells.

The modifications to a bacteriophage (or phage) genome can be provided either as single (e.g., one-at-a time) modifications or multiple changes can be introduced into the phage genomes simultaneously. The modifications to the phage genome can be introduced by components harbored in at least one bacterial host, including a reverse-transcribed editing template, which edits the phage genome during replication.

The bacteriophages can be modified in one type of bacterial host cells or by more than one type of bacterial host cells. In some cases, the modifications to the phage genome can be introduced by a single mixed culture of bacterial host cells, each host cell having one or more types of reverse-transcribed editing templates. In other cases, the modifications to the phage genome can be introduced by incubating phage through a series of host cell populations, where the host cell populations have different reverse-transcribed editing templates. For example, the phage do not need to be removed after one cycle of editing. Instead, the phage can be incubated with host cells through several cycles of editing. One or more populations or types of bacterial hosts can therefore be used that harbor different editing components to edit the phage genome at multiple locations. Additional host cells can be added to the phage/host cell mixtures if desired or needed. For example, the phage can be incubated through several cycles of editing in a single population or a mixed population of host cells. In some cases, phages propagated through one host cell population can be isolated and then sequentially introduced to a series of bacterial host cell populations to generate multiple engineered modifications at distinct, even separate locations on their genome. However, such isolation may not be necessary or needed.

By propagating phage through these editing hosts, the proportion of edited phages can build up over time, enabling high-efficiency phage genome engineering. Use of the methods for editing phage genomes as described herein provides important tools for expanding the utility of phage in biotechnology (e.g., for the delivery of molecular cargo to cells) or as engineered antimicrobials for therapeutic or industrial uses.

Hence, described herein are methods that involve incubating a population of bacteriophages through at least one bacterial host cell population to generate a population of genomically edited bacteriophages. The methods can include incubating the bacteriophages in a mixed population of bacterial host cells and/or through several rounds of editing. Such methods can generate populations of phages with higher percentages of genomic edits and/or a greater diversity of genomic edits than a control population of bacteriophages incubated for just one cycle of editing in a bacterial host cell population (e.g., through one lytic cycle).

The bacterial host cells can have one or more recombinantly expressed types of retron non-coding RNAs (ncRNAs), each encoding one or more donor DNAs having at least one segment adapted for editing phage genomes, as well as one or more endogenously or recombinantly expressed reverse transcriptases. Each bacterial host cell population can also include one or more types of endogenously or recombinantly expressed single strand annealing proteins (SSAPs, e.g., one or more RecT recombinases), single-stranded DNA binding proteins (SSBs), mutant mismatch repair proteins, or combinations thereof. Use of guide RNAs and the cas nucleases (often used for editing cellular genomes) are generally not needed or desired for editing phage genomes.

Hence described herein are bacterial host cells and bacterial host cell populations that include: (a) one or more recombinantly expressed types of retron non-coding RNAs (ncRNAs), each ncRNA encoding at least one donor DNA adapted for editing phage genomes; and (b) one or more recombinantly expressed reverse transcriptases. The bacterial host cells and populations of bacterial host cells can also include at least one endogenously or recombinantly expressed single strand annealing protein (SSAP, e.g., a RecT recombinase), single-stranded DNA binding protein (SSB), mutant mismatch repair protein, or a combinations thereof.

The ncRNAs that can be employed and that are modified, can be modified versions of one or more types of retron nucleic acids (e.g., where the retrons are named by various naming systems as Eco1, Eco2, Ec48, E67, Ec73, Ec78, EC83, EC86, EC107, Ec107, Mx65, Mx162, Sa163, Vc81, Vc95, Vc137, Vc96, or Ne144; combinations of such retron nucleic acids can also be used).

### DESCRIPTION OF THE FIGURES

FIG. 1A-1B illustrate the components and methods for genomic editing of bacteriophages. FIG. 1A is a schematic of the phage genome editing system in which a retron reverse-transcribed DNA template produced in a bacterial host is used to introduce a precise modification to the phage genome via recT-mediated recombination. FIG. 1B is a schematic diagram illustrating multiplexed editing in which phage is propagated through a mixed population of multiple bacterial strains, each harboring components as illustrated in FIG. 1A. to edit different sites on the phage genome. Over time these mutations accumulate on individual phage genomes.

FIG. 2 graphically illustrates the percent of lambda phage genomes containing a precise edit to the tail tip L gene (A698G) after 16 hours of culture through bacteria expressing the retron ncRNA encoding the edit, along with a retron reverse transcriptase (RT, from Eco1), CspRecT, and mutL E32K. The CspRecT is a RecT protein from Collinsella stercoris phage. As illustrated, the precise edits obtained depended on reverse transcription of the ncRNA, because such editing did not occur in a system expressing a mutated (catalytically dead) reverse transcriptase (dead RT). The editing was also improved by expression of recT in the host cells-little or no editing occurred in the absence of recT expression.

FIG. 3A-3B illustrate use of additional rounds of infection for editing of two sites in lambda phage within a single bacterial host, where a single editing ncRNA was used that contained editing templates for two nearby, but not directly adjacent, sites in the phage genome. FIG. 3A graphically illustrates editing rates for either of the two edits alone, or for the two edits provided by the retron ncRNA. Phage was propagated through freshly induced bacterial cultures harboring the editing components three times to demonstrate that the proportion of edited phage genomes increases with additional rounds of infection. FIG. 3B illustrates verification of edited, infectious phage sequences as detected by Sanger sequencing of individual phage plaques isolated after serial incubations in host cells to allow several rounds of editing.

FIG. 4 illustrates editing of two sites in lambda phage by two independent bacterial strains (strain 1 and strain 2), each expressing a different retron ncRNA. Phage were independently edited by strain 1 or strain 2 when separately propagated alone only through one of the two strains. When the strains are mixed together and phage is propagated through the mixed culture, both edits are made in individual phage genomes. The proportion of doubly edited phage genomes in practice is nearly identical to the predicted rate based on the editing rate of each strain alone.

FIG. 5 illustrates editing of T5 phage genome as a function of phage multiplicity of infection (MOI) at the onset of culture infection.

FIG. 6A-6B illustrate editing of T2 (a) and T7 (b) phages as a function of reverse transcribed editing template direction. FIG. 6A graphically illustrates editing of T2 phages as a function of reverse transcribed editing template direction. FIG. 6B graphically illustrates editing of T7 phages as a function of reverse transcribed editing template direction.

FIG. 7A-7G illustrate various retron ncRNA/RT-DNA structural features as well as retron similarities and differences. FIG. 7A shows a schematic of Eco1 and Eco4 ncRNAs, illustrating a difference between them in the loop identified as having positions 1-3. Both have a1/a2 and stem-loop regions that can be modified as described herein (the a1/a2 regions are labeled and the stem-loop regions are shown in blue). FIG. 7B illustrates the relative abundance of RT-DNA from Eco1 variants having modified loop bases at positions 1-3 of the loop shown in FIG. 7A. Deeper red shades indicate more RT-DNA production. As illustrated, use of thymine in the DNA encoding the ncRNA (before expression), or uracil in the ncRNA, at position 2 in the loop indicated in FIG. 7A of the Eco1 retron ncRNA provides improved RT-DNA (donor DNA) production. FIG. 7C illustrates the relative abundance of RT-DNA from Eco4 variants having modified loop bases at the positions indicated in FIG. 7A. Deeper red shades indicate more RT-DNA production. As illustrated, use of thymine in the DNA encoding the ncRNA, or uracil in the ncRNA, at position 2 in the loop indicated in FIG. 7A does not improve RT-DNA (donor DNA) production in the Eco4 ncRNA as much as it does for the Eco1 ncRNA. Eco4 is less vulnerable than Eco1 to sequence alterations in this loop. FIG. 7D graphically illustrates the relative RT-DNA abundance produced from different Eco1 stem length variants analyzed, where the RT-DNA abundance is shown as a percentage of wild-type abundance (dashed line). As illustrated, the RT-DNA abundance varies depending upon the length of the stem up to about stem length fifteen. FIG. 7E graphically illustrates the relative RT-DNA abundance of different Eco1 a1/a2 stem length variants as a percentage of wild-type abundance (dashed line). FIG. 7F graphically illustrates the relative RT-DNA abundance of each Eco4 stem length variant as a percentage of wild-type abundance (dashed line). FIG. 7G graphically illustrates the relative RT-DNA abundance of different Eco4 a1/a2 length variants as a percentage of wild-type abundance (dashed line).

FIG. 8A-8B illustrate modified retron structural elements that can be used in genome editing. FIG. 8A shows a schematic of a ncRNA having a RT-DNA template that could be used for phage editing. The retron ncRNA is modified in the msd region (blue) to include a long loop (green) that contains a region encoding a DNA donor sequence with homology to a genomic locus, but where the DNA donor sequence has one or more nucleotide modifications (repair nucleotides; asterisks). Such an ncRNA therefore provide a template for a donor DNA that is made by reverse transcription. FIG. 8B graphically illustrates fold enrichment of reverse transcribed DNA (RT-DNA) when an Eco1-based retron ncRNA has a longer stem (a1/a2) region of 22 nucleotides, compared to just 12 nucleotides. The RT-DNA products were detected using qPCR, with the RT-DNA from each construct shown relative to uninduced. Circles show each of three biological replicates, with black for the wild type a1/a2 length and green for the extended a1/a2. This experiment was performed using procedures like those used for the data obtained for FIGS. 7E and 7G. See FIG. 7 for location of the retron a1/a2 region.

FIG. 9 illustrates some structural features of retrons that can be modified in a library of retrons.

FIG. 10A-10H. Recombitrons Target Phage Genomes for Continuous Editing A. Recombitron schematic: a modified retron generates ssDNA containing a donor sequence with an edit flanked by homology to the phage genome that is integrated into the phage genome during replication by SSB and SSAP. The retron cassette is expressed from an operon containing a reverse transcriptase (RT) and ncRNA. The reverse-transcribed region of the ncRNA is shown in purple with an inserted donor sequence in light blue, and the edit site is shown in orange. A second operon expresses CspRecT and mutL E32K. B. Left: Edited phage genomes (as a % of all genomes) across lambda phage. Editing with a forward RT-DNA is shown in blue, reverse in purple. Three individual replicates are shown in open circles at each point. Right: Editing with the recombitron at site 14,126 (±SD) is significantly greater than editing with a dRT control (t-test, P=0.0018). C. Left: Edited phage genomes across phage T7 for three replicates at each position, displayed as in b. Right: Editing with the recombitron at site 22,872 (±SD) is significantly greater than editing with a dRT control (t-test, P=0.0094). D. Left: Edited phage genomes across phage T5 for three replicates at each position, displayed as in B. Right: Editing with the recombitron at site 27,182 (±SD) is significantly greater than editing with a dRT control (t-test, P<0.0001). E. Editing of lambda at site 30,840 (F) (±SD) compared to editing with supplemental expression of E. coli SSB or T7 SSB. There is a significant effect of SSB expression (one-way ANOVA, P<0.0001, N=3), with both the E. coli (P=0.005) and T7 (P=<0.0001) significantly different than the no SSB condition (Dunnett's, corrected). F. Editing of T7 at site 11,160 (R) (±SD) compared to editing with supplemental expression of E. coli SSB or T7 SSB. There is a significant effect of SSB expression (one-way ANOVA, P<0.0001, N=3), with both the E. coli (P=0.0002) and T7 (P=0.0127) significantly different than the no SSB condition (Dunnett's, corrected). G. Schematic illustrating the accumulation of edited phages with multiple rounds of editing. H. Proportion of edited lambda phage increases over 3 rounds of editing of lambda at site 30,840 (F). Three replicates per round are shown in open circles.

FIG. 11A-11H. Accompaniment to FIG. 10. A. Left: Edited phage genomes (as a % of all genomes) across phage T2. Editing with a forward RT-DNA is shown in blue, reverse in purple. Three individual replicates are shown in open circles at each point. Right: Editing with the recombitron at site 143,279 (R) (±SD) versus a dRT control. B. Editing (%) of lambda and lambda_ΔcI in different host strains at site 14,070 (R). Mean±SD is shown for 3 (wt in bSLS.114), 5 (ΔcI in bSLS.114), and 2 (ΔcI in bCF.5) biological replicates. C. Editing (%) of lambda without induction of CspRecT. Mean±SD for 3 biological replicates at site 14,126. D. Titer (PFU/mL) of phage lambda, T7, and T5 after propagation through host cells of different conditions, compared to amount of phage added to the culture. Host conditions are without recombitrons (open black circles), with uninduced recombitrons (open pink circles), with induced recombitrons (closed pink circles), and with induced recombitrons that target a different phage (closed blue circles). Individual biological replicates are shown in circles for each condition. E. Editing (%) of phage lambda, T7, and T5 from the induced, on-target recombitron condition in panel D. F. Editing (%) of lambda at site 14126 (F) compared to editing with supplemental expression of T5 SSB. G. Editing (%) of T7 at site 22872 (R) compared to editing with supplemental expression of T5 SSB. H. Editing (%) of T5 at site 88634 (F) with supplemental expression of E. coli SSB, T7 SSB, or T5 SSB.

FIG. 12A-12J. Optimizing Recombitron Parameters with Lambda Phage. A. Schematic of recombitron with longer and shorter RT-Donors, with more or less homology to the genome surrounding the edit site. B. Editing (%) of lambda with donors ranging in size from 30-150 bases. Three to four biological replicates are shown in open circles and closed circles show the average per recombitron, demonstrating an effect of length (one-way ANOVA, P<0.0001). All RT-Donors greater than 50 bases yield significantly improved editing from a 30 base donor (Sidak's corrected, P<0.05), and a RT-Donor of 150 bases is significantly worse than 70 bases (Sidak's corrected, P<0.05). C. Schematic of RT-Donors with homology to a single lambda region, each containing a scanning edit. D. Editing (%) of lambda with donors containing scanning edit. Four biological replicates are shown in open circles and closed circles show the average per recombitron, demonstrating an effect of edit placement (one-way ANOVA, P<0.0001). Edit placement at positions ≤28 or ≥73 is significantly worse position 46 (Dunnett's corrected, P<0.001). E. Schematic of RT-Donors containing the scanning edit with an additional, constant central edit. F. Editing (%) of lambda with donors containing both the scanning and central edits. Editing rate when only the central edit is acquired is shown in light pink, acquisition of both edits shown in purple (±SD). Position of scanning edit does not affect total editing (one-way ANOVA, P=0.7868), but does affect whether both edits are installed (one-way ANOVA, P<0.0001). G. Schematic of multi-edit recombitrons containing 3, 7, 9, and 11 edits per RT-Donor. H. Editing (%) of lambda from the multi-edit recombitrons, each site across three to four biological replicates is shown as an open circle. Number of edits per RT-Donor significantly affects average editing rate across sites (one-way ANOVA, P<0.0001), with 7, 9, and 11 edits all performing significantly worse than 3 (Dunnett's corrected, P<0.0001). I. Co-occurrence of edits that are acquired from multi-edit donors. Heatmap shows correlation coefficient r\hat{ }2, normalized to the overall editing rate for each donor at each site. J. Editing (%) of lambda in different host strains. Three biological replicates are shown as open circles and the average is shown as a closed circle. There is a significant effect of strain (one-way ANOVA, P<0.0001). The MG1655 (exol-/recJ-) strain yielded significantly more editing than the BL21 (Dunnett's corrected, P<0.0001).

FIG. 13. Accompaniment to FIG. 12. Rate of acquiring only the scanning edit in lambda when donors contain both scanning and central edits. (mean±SD).

FIG. 14A-14F. Insertions and Deletions via Recombitrons. A. Schematic of phage genome deletions of increasing size around a common central site (pink hexagon). B. Quantification of editing efficiency for deletions preceding position 37,673 as compared to a single base pair synonymous substitution. Five biological replicates are shown in open circles and the mean is shown as a closed circle. There is no significant effect of deletion length (one-way ANOVA, P=0.1564), but all deletions occur at a significantly lower frequency than the single-base substitution (one-way ANOVA, P<0.0001; Dunnett's corrected vs 2 P<0.0001 vs 4 P=0.0083 vs 8 P=0.0008 vs 16 P<0.0001 vs 32 P=0.0005). C. Schematic of phage genome insertions of increasing size ending at a common position (pink hexagon). D. Quantification of editing efficiency for insertions at position 37,673 as compared to a single base pair synonymous substitution. Five biological replicates are shown in open circles and the mean is shown as a closed circle. Note that the same single base substitution was used as a parallel control for both deletions and insertions and thus the same points are shown here as in B. There is a significant effect of insertion length (one-way ANOVA, P<0.0001), and all insertions occur at a significantly lower frequency than the single-base substitution (one-way ANOVA, P<0.0002; Dunnett's corrected P<0.0001 for all insertions). E. Schematic illustrating long-read nanopore quantification. F. Quantification of editing efficiency using nanopore sequencing. Points shown represent the fraction of edited reads pooled across three biological replicates. 64 and 100 base pair deletions were not detected in 57 and 102 reads aligned to the edit region, respectively.

FIG. 15A-15E. Accompaniment to FIG. 14. A. Comparison of edited phages measure by amplicon (Illumina) or amplification-free (Oxford Nanopore) sequencing. Open orange circles represent biological replicates of amplicon data and filled orange circle represents the mean. Filled blue circle represents the aggregate nanopore data from three replicates. B. Coverage of the editing site in long-read nanopore sequencing for deletions in which we observe editing. C. Coverage of the editing site in long-read nanopore sequencing for deletions in which we do not observe any edits. Estimated limit of detection for these samples is calculated by dividing 100 by the coverage of the site. D. Coverage of the editing site in long-read nanopore sequencing for large insertions, for which we do not observe any edits. Estimated limit of detection for these samples is calculated by dividing 100 by the coverage of the site. E. Examples of nanopore reads for different deletion conditions.

FIG. 16A-16J, Multiplexed Phage Engineering via Recombitrons A. Schematic illustrating the propagation of phages through cells expressing distinct recombitrons in a co-culture. Over generations of phage propagation, phage genomes accumulate multiple edits from different RT-Donors. B. Editing (%) increases over three rounds of editing. Editing in engineered K-strain E. coli outperforms the B-strain (two-way ANOVA, strain P<0.001, round P<0.0001, interaction P=0.0282). Three biological replicates are shown in open circles, lines connect averages. C. Editing (%) of each site from mixed recombitron cultures after 1 round of editing. Three biological replicates are shown in open circles for each site, clustered over the number of recombitrons used. D. Editing (%) of each site from mixed recombitron cultures after 3 rounds of editing, shown as in C. E. Editing (%) of each site after 1 round of editing in relation to the proportion of that recombitron strain in the culture (%) (two-tailed Pearson correlation, P<0.0001). F. Average editing (%) of all sites in each mixed culture. Points represent mean of three biological replicates ±SD (two-way ANOVA, number of editors P<0.0001, round P<0.0001, interaction P=0.234) G. Schematic illustrating Sanger sequencing of plaques, which enables quantification of each editing site from clonal phages. Representative data from one plaque with five edits (yellow) and two wild-type sites (grey). H. Overall editing (%) of any site on plaques isolated from mixed cultures. Points represent mean of three biological replicates ±SD. I. Number of edits made on a single phage genome from mixed cultures, by number of recombitrons. Individual plaques from three biological replicates are shown as open circles on a violin plot of distribution. J. Number of edits made on a single phage genome from mixed cultures, by number of recombitrons, shown as a histogram (mean±SD).

FIG. 17. Accompaniment to FIG. 16. Editing (%) from Sanger sequencing of plaques at each site from mixed recombitron cultures after 3 rounds of editing. Three biological replicates are shown in open circles for each site, clustered over the number of recombitrons used.

### DETAILED DESCRIPTION

Methods for editing bacteriophages (phages) are described herein. Use of modified bacteriophages can be beneficial compared to current uses for antibiotics. For example, phages can be effective against both treatable and antibiotic-resistant bacteria, phages multiply and increase in number by themselves during treatment so only one dose may be needed, phages are specific to particular bacteria, phages may not or may only slightly disturb normal “good” bacteria in the body, phage can be used alone or with other drugs (including antibiotics), phages are readily available, phages are not harmful or toxic to the body, and phages are not toxic to animals, plants, and the environment.

However, many bacteria have defense mechanisms that inhibit or prevent currently available bacteriophages from killing them. The methods described herein can improve the targeting of bacteriophages and allow the bacteriophages to escape anti-phage defenses of targeted bacteria. These methods can include infecting at least one population of bacterial host cells with phages, where the bacterial host cells have been modified to provide donor DNAs for genomic editing such as modified retron nucleic acids. Such editing can be targeted to sites in the phage genome that make them vulnerable to bacterial defense systems.

In some cases, a series of bacterial host cells can be infected with phages that have been generated (and potentially edited) by one or more previous rounds of incubation in the bacterial host cells that were designed for such phage editing. Each round of infection/editing can add new genomic edits and/or increase the percentage of edited phages in the phage population.

The bacterial host cells can be any bacterial cells that have phage receptor binding proteins (RBP) for the phage type(s) of interest and that provide an environment where the phages can replicate. Hence, many types of bacterial host cells can be used. However, in some cases, the bacterial host cells include bacteria involved in bacterial infections, antibiotic-resistant bacteria, bacteria from an ongoing infection, or combinations thereof. Phage can be introduced into such pathogenic bacterial host cells, for example, to evaluate the effects of the phage on the host cells, or to evaluate whether the phage are robust enough (e.g., modified sufficiently) to infect and replicate in such host cells. Phage can be introduced into such pathogenic bacterial host cells, for example, to evaluate whether the phage can kill the pathogenic bacteria. Hence, phage populations can be identified that may need additional editing or that are useful for killing their bacterial host cells.

The bacterial host cells are modified to provide retron nucleic acids that encode one or more donor DNAs adapted for editing phage genomes. Multiple copies of the donor DNAs are generated within the cells by reverse transcription of at least one type of retron non-coding RNA (ncRNA). Hence, bacterial host cells are used that endogenously or recombinantly express a reverse transcriptase. The bacterial host cells can also include components to facilitate editing of the phage genomes, including one or more types of single strand annealing proteins (SSAPs), single-stranded DNA binding proteins (SSBs), mismatch repair (e.g., mutL) mutants, or combinations thereof. In some cases, the bacterial host cells can be modified to include a dominant-negative mutant mutL gene (e.g., with an E32K mutation). For example, one or more types of single strand annealing proteins (SSAPs) can help the donor editing DNA anneal to the phage genome during replication, thereby transferring the desired edits to the replicated phage genome.

Hence, guide RNAs, tracrRNAs, and cas nucleases are not needed for editing phage genomes.

Multiple copies of donor DNAs can be generated in vivo from retron templates. For example, modified retron non-coding RNAs (ncRNAs) can be expressed from an expression cassette within the host cells as RNA molecules. Retron ncRNAs are naturally partially reverse transcribed into ssDNA. As provided herein, the portion of the ncRNA that is partially reverse transcribed can provide the donor DNA for editing the phage genomes. Such reverse transcription provides multiple copies of single stranded donor DNA, which is ideal for editing phage genomes during phage replication.

The donor DNAs can be generated in host cells that also provide one or more types of single strand annealing proteins (SSAPs) and/or one or more single-stranded DNA binding proteins (SSBs). The SSAPs can facilitate recombination (editing) and in some cases the SSAP is a RecT recombinase. Single-stranded DNA binding proteins (SSBs) bind and stabilize single-stranded DNA (ssDNA). The SSAP and/or SSB proteins can be expressed endogenously or the bacterial host cells can be modified to include an expression cassette from which the SSAP and/or SSB proteins can be expressed. For example, in some cases the bacterial host cells can have, or be modified to express CspRecTas a SSAP. RecT binds to single-stranded DNA and promotes the renaturation of complementary single-stranded DNAs to facilitate recombination. RecT has a function similar to that of lambda RedB.

Constructs can also be used to express the different ncRNAs, reverse transcriptases, along with the SSAP, SSB, mutant mismatch repair proteins (e.g., mutL mutants), or combinations thereof. Any of the constructs or expressed nucleic acids can be linked to a barcode. For example, a linked bar code can be inserted into the phage genome along with donor DNA. Such barcodes can facilitate evaluation of the genomic edits within the phage genomes. Segments of DNA with the bar code, for example, can be recovered and evaluated by sequencing. Barcodes can provide primer sites for sequencing and can identify the type of genomic edit that was intended to be made (e.g., allowing comparison with the target site sequence to assess the fidelity of the editing system).

### Bacterial Host Cells

As mentioned above, the bacterial host cells can be any bacterial cells that have phage receptor binding proteins (RBP) for the phage type(s) of interest. However, bacteriophages can be species-specific with regard to their hosts and may only infect a single bacterial species or even some specific strains within a species.

For example, in some cases the bacterial hosts include Escherichia coli. However, other bacterial species can be used as host cells. For example, the bacterial host cells can be one or more strains of Escherichia coli (often linked to gastrointestinal distress), Salmonella (often linked to food poisoning), Mycobacterium (causes tuberculosis), Bacillus anthracis (anthrax), Citrobacter freundii (gastroenteritis, neonatal meningitis, and septicemia), Clostridium tetani (tetanus), Clostridium botulinum (botulism), Clostridium difficile (gastrointestinal problems, especially in those with a weak immune system), Enterobacter hormaechei (nosocomial infections), Haemophilus influenzae (meningitis), Haemophilus influenzae Type B (ear, throat, lung infections), Heliobacter pylori (stomach ulcers), Klebsiella pneumoniae (infections, especially lung infections), Leptospira (Leptospirosis), Listeria monocytogenes (meningitis), Pseudomonas aeruginosa (infections, especially lung infections), Neisseria gonorrhoeae (gonorrhea), Neisseria meningitidis (meningitis), Serratia marcescens (nosocomial infections), Shigella dysenteriae (dysentery, shigellosis), Staphylococcus aureus (e.g., methicillin-resistant Staphylococcus aureus (MRSA) that is resistant to antibiotics), Streptococcus pneumoniae (infections, especially lung infections, and meningitis), Treponema pallidum (syphilis), Vibrio cholerae (cholera), Vibrio vulnificus (“flesh-eating” bacteria), Yersinia pestis (bubonic plague), Legionella pneumophila (Legionnaires' disease), Cutibacterium acnes (acne) and others.

The bacterial host cells can be modified to include retron nucleic acids (ncRNAs) that encode donor DNAs adapted for editing phage genomes.

The bacterial host cells can also include components to facilitate editing of the phage genomes, including one or more types of reverse transcriptases, single strand annealing proteins (SSAPs), single-stranded DNA binding proteins (SSBs), mismatch repair (e.g., mutL) mutants, or combinations thereof. In some cases, the bacterial host cells can be modified to include a dominant-negative mutant mutL gene (e.g., with an E32K mutation). Sequences for such protein components are available in the database provided by National Center for Biotechnology Information (NCBI, see website at ncbi.nlm.nih.gov). Some examples of sequences for these types of proteins are provided herein but other sequences for these types of proteins can also be used.

A variety of one or more single strand annealing proteins (SSAPs) can be expressed, either endogenously or recombinantly, to facilitate recombination during editing of the phage's genomes. In general, the one or more single strand annealing proteins (SSAPs) so expressed are compatible with one or more single-stranded binding proteins (SSBs) to promote recombination during editing. The SSAPs can be bacterial or phage SSAPs-either the bacterial host cell or the infecting phage can express such SSAPs.

For example, one type of SSAP that can be expressed by bacteria during editing of phage genomes is the bacteriophage lambda bet (also called RedB) SSAP protein that has the following protein sequence (NCBI NP_040617.1; SEQ ID NO: 1).

| | |
|---|---|
| 1 | MSTALATLAG KLAERVGMDS VDPQELITTL RQTAFKGDAS |
| 41 | DAQFIALLIV ANQYGLNPWT KEIYAFPDKQ NGIVPVVGVD |
| 81 | GWSRIINENQ QFDGMDFEQD NESCTCRIYR KDRNHPICVT |
| 121 | EWMDECRREP FKTREGREIT GPWQSHPKRM LRHKAMIQCA |
| 161 | RLAFGEAGIY DKDEAERIVE NTAYTAERQP ERDITPVNDE |
| 201 | TMQEINTLLI ALDKTWDDDL LPLCSQIFRR DIRASSELTQ |
| 241 | AEAVKALGFL KQKAAEQKVA A |

An example of a protein sequence for a Shigella dysenteriae bet SSAP protein is shown below (NCBI AAF28115.1; SEQ ID NO:2).

| | |
|---|---|
| 1 | MSTALATLAG KLAERVGMDS VDPQELITTL RQTAFKGDAS |
| 41 | DAQFIALLIV ANQYGLNPWT KEIYAFPDKQ NGIVPVVGVD |
| 81 | GWSRIINENQ QFDGMDFEQD NESCTCRIYR KDRNHPICVT |
| 121 | EWMDECRRAP FKTREGREIT GPWQSHPKRM LRHKAMIQCA |
| 161 | RLAFGFAGIY DKDEAERIVE NTAYTTERQP ERDITPVNEE |
| 201 | TMSEINALLT FMEKTWDDDL LPLCSQIFRR NIYTSSELTQ |
| 241 | AEAVKVLGFL KQKVTEQKVA A |

An example of a protein sequence for an Escherichia phage Stx2 II bet SSAP protein is shown below (NCBI NP_859351.1; SEQ ID NO:3).

| | |
|---|---|
| 1 | MSTALATLAG KLAERVGMDS VDPQELITTL RQTAFKGDAS |
| 41 | DAQFIALLIV ANQYGLNPWT KEIYAFPDKQ NGIVPVVGVD |
| 81 | GWSRIINENQ QFDGMDFEQD NESCTCRIYR KDRNHPICVT |
| 121 | EWMDECRREP FKTREGREIT GPWQSHPKRM LRHKAMIQCA |
| 161 | RLAFGFAGIY DKDEAERIVE NTAYTAERQP ERDITPVNDE |
| 201 | TMQEINTELI ALDKTWDDDL LPLCSQIFRR DIRASSELTQ |
| 241 | AEAVKVLGFL KQKASEQKVA A |

An example of a protein sequence for an Enterobacteria phage VT2-Sakai bet SSAP protein is shown below (NCBI BAA84297.1; SEQ ID NO:4).

| | |
|---|---|
| 1 | MSTALATLAG KLAERVGMDS VDPQELITTL RQTAFKGDAS |
| 41 | DAQFIALLIV ANQYGLNPWT KEIYAFPDKQ NGIVPVVGVD |
| 81 | GWSRIINENQ QFDGMDFEQD NESCTCRIYR KDRNHPICVT |
| 121 | EWMDECRREP FKTREGREIT GPWQSHPKRM LRHKAMIQCA |
| 161 | RLAFGFAGIY DKDEARRIVE NTAYTAERQP ERDITPVNDE |
| 201 | TMQEINTILI ALDKTWDDDL LPLCSQIFRR DIRASSELTQ |
| 241 | AEAVKVLGFL KQKASEQKVA A |

An example of a protein sequence for an Escherichia phage vB_EcoP_24B bet SSAP protein is shown below (NCBI ADN68402.1; SEQ ID NO:5).

| | |
|---|---|
| 1 | MSTALATLAG KLAERVGMDS VDPQELITTL RQTAFKGDAS |
| 41 | DAQFIALLIV ANQYGLNPWT KEIYAFPDKQ NGIVPVVGVD |
| 81 | GWSRIINENQ QFDGMDFEQD NESCTCRIYR KDRNHPICVT |
| 121 | EWMDECRREP FKTREGREIT GPWQSHPKRM LRHKAMIQCA |
| 161 | RLAFGFAGIY DKDEAERIVE NTAYTAERQP ERDITPVNDE |
| 201 | TMQEINTILI ALDKTWDDDL LPLCSQIFRR DIRASSELTQ |
| 241 | AEAVKALGFL KQKATEQKVA A |

An example of a protein sequence for a Serratia phage Eta recombination (bet) protein is shown below (NCBI YP_008130312.1; SEQ ID NO:6).

| | |
|---|---|
| 1 | MSTALAAIAQ SSGVSVDDVT DVLKGMIISA KNQHGAQVSN |
| 41 | AELAVVSGVC AKYDLNPMVK ECAAFISGGK LQVVLMIDGW |
| 81 | YRIVNRQPNF DGVEFDDHID DKSVLTAITC RMYIKGRTRP |
| 121 | VVVTEYMSEC RDPKSSVWQK WPARMLRHKA YIQCARMTFG |
| 161 | ISDMIDNDEA SRITQGEKNI TQQASSVSTV DYQAIDQAMG |
| 201 | ECEDHDALNK LCAEIRAEME KRGTWNSEKV TLADMKSRHK |
| 241 | ARIDAAVVTD EFEVVEDDND GAVKSDVEDS ATDDDVPFE |

In some cases, the SSAPs expressed by the bacterial host cells include a RecT recombinase. Such recombination facilitating RecT proteins are of the Pfam family: PF03837. One example of a RecT protein sequence is the following Enterobacteriaceae RecT protein sequence (NCBI WP_000166319.1; SEQ ID NO:7).

| | |
|---|---|
| 1 | MTKQPPIAKA DLQKTQGNRA PAAVKNSDVI SFINQPSMKE |
| 41 | QLAAALPRHM TAERMIRIAT TEIRKVPALG NCDTMSFVSA |
| 61 | IVQCSQLGLE PGSALGHAYL LPFGNKNEKS GKKNVQLIIG |
| 121 | YRGMIDLARR SGQIASLSAR VVREGDEFSF EFGLDEKLIH |
| 161 | RPGENEDAPV THVYAVARLK DGGTQFEVMT RKQIELVRSL |
| 201 | SKAGNNGPWV THWEEMAKKT AIRRLFKYLP VSIEIQRAVS |
| 241 | MDEKEPLTID PADSSVLTGE YSVIDNSEE |

A nucleotide sequence that encodes an Enterobacteriaceae RecT protein is shown below (NCBI NC_000913.3; SEQ ID NO:8).

| | |
|---|---|
| 1 | ATGACTAAGC AACCACCAAT CGCAAAAGCC GATCTGCAAA |
| 41 | AAACTCAGGG AAACCGTGCA CCAGCAGCAG TTAAAAATAG |
| 61 | CGACGTGATT AGTTTTATTA ACCAGCCATC AATGAAAGAG |
| 121 | CAACTGGCAG CAGCTCTTCC ACGCCATATG ACGGCTGAAC |
| 161 | GTATGATCCG TATCGCCACC ACAGAAATTC GTAAAGTTCC |
| 201 | GGCGTTAGGA AACTGTGACA CTATGAGTTT TGTCAGTGCG |
| 241 | ATCGTACAGT GTTCACAGCT CGGACTTGAG CCAGGTAGCG |
| 281 | CCCTCGGTCA TGCATATTTA CTGCCTTTTG GTAATAAAAA |
| 321 | CGAAAAGAGC GGTAAAAAGA ACGTTCAGCT AATCATTGGC |
| 361 | TATCGCGGCA TGATTGATCT GGCTCGCCGT TCTGGTCAAA |
| 401 | TCGCCAGCCT GTCAGCCCGT GTTGTCCGTG AAGGTGACGA |
| 441 | GTTTAGCTTC GAATTTGGCC TTGATGAAAA GTTAATACAC |
| 481 | CGCCCGGGAG AAAACGAAGA TGCCCCGGTT ACCCACGTCT |
| 521 | ATGCTGTCGC AAGACTGAAA GACGGAGGTA CTCAGTTIGA |
| 561 | AGTTATGACG CGCAAACAGA TTGAGCTGGT GCGCAGCCTG |
| 601 | AGTAAAGCTG GTAATAACGG GCCGTGGGTA ACTCACTGGG |
| 641 | AAGAAATGGC AAAGAAAACG GCTATTCGTC GCCTGTTCAA |
| 681 | ATATTTGCCC GTATCAATTG AGATCCAGCG TGCAGTATCA |
| 721 | ATGGATGAAA AGGAACCACT GACAATCGAT CCTGCAGATT |
| 761 | CCTCTGTATT AACCGGGGAA TACAGTGTAA TCGATAATTC |
| 810 | AGAGGAATAA |

Another example of a RecT protein sequence is the following Escherichia coli RecT protein sequence (NCBI QTN08202.1; SEQ ID NO:9).

| | |
|---|---|
| 1 | MTKQPPIAKA DLQKTQGNRA PAAVKNSDVI SFINQPSMKE |
| 41 | QLAAALPRHM TAERMIRIAT TEIRKVPALG NCDTMSFVSA |
| 81 | IVQCSQLGLE PGSALGHAYL LPFGNKNEKS GKKNVQLIIG |
| 121 | YRGMIDLARR SGQIASLSAR VVREGDEFSF EFGLDEKLIH |
| 161 | RPGENEDAPV THVYAVARLK DGGTQFEVMT RKQIELVRSL |
| 201 | SKAGNNGPWV THWEEMAKKT AIRRLFKYLP VSIEIQRAVS |
| 241 | MDEKEPLTID PADSSVLTGE YSVIDNSEE |

An example of an Escherichia ruysiae RecT sequence is shown below (NCBI MBY7351797.1; SEQ ID NO:10).

| | |
|---|---|
| 1 | MTKQPPIAKA DLQKTQGNRA PAAVKNSDVI SFINQPSMKE |
| 41 | QLAAALPRHM TAERMIRIAT TEIRKVPALG NCDTMSFVSA |
| 81 | IVQCSQLGLE PGSALGHAYL LPFGNKNEKS GKKNVQLIIG |
| 121 | YRGMIDLARR SGQIASLSAR IVREGDEFSF EFGLDEKLIH |
| 161 | RPGENEDAPV THVYAVARLK DGGTQFEVMT RKQIELVRSL |
| 201 | SKAGNNGPWV THWEEMAKKT AIRRLFKYLP VSIEIQRAVS |
| 241 | MDEKEPLTID PADSSVLTGE YSVIDNSEE |

An example of a Salmonella RecT protein sequence is shown below (NCBI WP_079839509.1; SEQ ID NO:11).

| | |
|---|---|
| 1 | MPKQPPIAKA DLQKTQGARP PTAVKNNNDV ISFINQPSMK |
| 41 | EQLAAALPRH MTAERMIRIA TTEIRKVPAL GDCDTMSFVS |
| 81 | AIVQCSQLGL EPGGALGHAY LLPFGNRNEK SGKKNVQLII |
| 121 | GYRGMIDLAR RSGQIASLSA RVVREGDDFS FEFGLEEKLV |
| 161 | HRPGENEDAP VTHVYAVARL KDGGTQFEVM TRKQIELVRA |
| 201 | QSKAGNNGPW VTHWEEMAKK TAIRRLFKYL PVSIEIQRAV |
| 241 | SMDEKETLTI DPADASVITG EYSVVENAGV EENVTA |

An example of a Mycobacterium tuberculosis RecT protein sequence is shown below (NCBI SGD85611.1; SEQ ID NO:12).

| | |
|---|---|
| 1 | MSNPPIAQAD LQKAQGTAVK EKTKDQQLIQ FINQPGMKAQ |
| 41 | LSAALPRHIT PDRMIRIVTT EIRKIPSLAT CDMQSFIGAV |
| 81 | VQCSQLGLEP GNALGHAYLL PFGNGKATSG QPNVQLIIGY |
| 121 | RGMIDLARRS GQIISISARS VREGDSFHFE YGLNEDLTHV |
| 161 | PGENDSGPIT HVYAVARLKE GGVQFEVMSF SQIEKVRDSS |
| 201 | KAGKNGPWVS HWEEMAKKTV IRRLFKYLPV SIEMQRAVIL |
| 241 | DEKAEANVDQ EHASIFEGEY ETVSPE |

An example of a Bacillus anthracis RecT protein sequence is shown below (NCBI GEU13688.1; SEQ ID NO:13).

| | |
|---|---|
| 1 | MSNDLTQITQ RSLDEQVIGN LNRLQEQGLE MPPGYSPQNA |
| 41 | LKSAFFELTN NSGGNLLQLA ANNPETKTSI SNALLDMVIQ |
| 81 | GLSPAKKQCY FIKYGNKVQL MRSYFGTMAV LDRVTGGAEI |
| 121 | TPVVVREGDV FEIAMDGPDL VVAKHETSFE NLDNDIKAAY |
| 141 | VVIKLANGKE VTTVMTKKQI DKSWSKAKTK NVQNDEPEEM |
| 201 | AKRTVINRAA KYLINTSNDN DLFVQAAKDT LENEFERKDV |
| 241 | TPEREEQTAV LEEKIFTNNK KVIEQENDIE RITRVADVPE |
| 281 | QPDIEQAKQI EKEDLTKVAD QILEEPVQET LDVMAGYETN |
| 321 | QKESEADVST IEEDDYPF |

An example of a Clostridium tetani RecT protein sequence is shown below (NCBI SUY55099.1; SEQ ID NO:14).

| | |
|---|---|
| 1 | MATNESLKNQ LTTKKETGLG SAGNTIKGLM NSPAIKKRFE |
| 41 | EVLKQRAPQY MSSIVNLVNS DINLKKCDQM SVVASCMVAA |
| 81 | TLDLPVDKNL GYAWVVPYGN KAQFQLGYKG YVQLALRTGQ |
| 121 | YKSINVIEIH EGELIDWNPL TEELKIDFSK KESDAVIGYA |
| 161 | GYFELLNGFK KSTYWTKEQI TKHKNKFSKS DFGWKKDFDA |
| 201 | MARKTVLRNM LSKWGILSIE MQNAYTADQG IIKNEIMETG |
| 241 | EVKENIEYIE ADFESYEDNS IEEGGANE |

An example of a Clostridium difficile RecT protein sequence is shown below (NCBI AXU84523.1; SEQ ID NO:15).

| | |
|---|---|
| 1 | MASEKAKGAL EKKVSGANTV KVSPSKGMEQ LMNKMASQIK |
| 41 | KALPSMVSSE RFQRVALTAF SNNPRLQSCE PMSFIAAMME |
| 81 | SAQLGLEPNT PLGQAYLIPY GNKVQFQIGY KGLLELAQRS |
| 121 | GKIKTIYAHK IRENDKFEIK YGLHQDLVHE PKLNGDRGEI |
| 161 | IGYYAVYHLD TGGHSFSFMT KEEIIEFAKS KSKSYSSGPW |
| 201 | QTDFDSMAKK TVIKQLLKYA PLSIELQKAM VGDETIKSEI |
| 241 | DEDMSMVVDE SESLEVDFEV KENMDGKVSV EEAINVD |

An example of a Haemophilus influenzae RecT-like single-stranded DNA binding protein is shown below (NCBI AJO88455.1; SEQ ID NO:16).

| | |
|---|---|
| 1 | MINQVQHQQN KQPPALKTFF ESANVQNKIK ELVGKNAATF |
| 41 | ATSVMQIANS NSMLKTADPM SIFNAACMAA TLNLPLQNGL |
| 81 | GFAYIVPFRN NKEKKTEAQF QIGYKGFIQL AQRSGQFKRL |
| 121 | VALPVYKKQL IKKDFINGFE FDWEQEPEQN ENPIGYYAYF |
| 161 | KLVNDFSAEL YMSHDDIVKH AQRYSQTFKK GYGVWHDNFE |
| 201 | AMALKTVTKL LLSKQAPLSV EMQQAVLADQ TVVKDVENQE |
| 241 | FNYTDNIQEA EFLAVVDEAT FEQCKQSIAN GETTLQELCD |
| 281 | SGAYEFSQEQ LTKLEELENQ KAE |

An example of a Staphylococcus aureus RecT protein sequence is shown below (NCBI BAV60852.1; SEQ ID NO:17).

| | |
|---|---|
| 1 | MTENNKLQTI EQQLVQEKNV SDNVLNKVRV LESQGNLELP |
| 41 | NDYSPSNAMK QAWLQISQDN KLMSCNDTSK ANALLDMVTQ |
| 81 | GLNPAKNQCY FIPYGNKMQL QRSYHGNVMM LKRDAGAQDV |
| 121 | VAQVIYKGDT FKQEMGGTGR IKAIKHEQDF FNIDKENIIG |
| 161 | AYCTIVFNDG RDNYIEVMTI EQIKQAWMQS SMIKDEKALQ |
| 201 | NSKTHNNFKE EMAKKTVINR AAKRYINTST DSNLFKYAQE |
| 241 | SEQRQRKEVL DAEVEENANQ EQLDFEQPVL EEAQYTELEN |
| 281 | DKPIDVSDFE EIKEPATEKE SEEEPF |

An example of a Streptococcus pneumoniae RecT protein sequence is shown below (NCBI VRD08895.1; SEQ ID NO:18).

| | |
|---|---|
| 1 | MANEIAKFDT LTPQQAFKSP AALEKFKSVL DGSETQFVAS |
| 41 | LLSIINNNSY LAQATNTSIM NAAMKAATLK LPIEPSLGMA |
| 81 | YVVPYNRSEK RGNTWVKINE AQFQMGYKGF IQLAQRSGQI |
| 121 | RNINCDVVYK EEFLRYDKVY GTLHLTDEQV DSGEVEGYFA |
| 161 | SLELINGFRK MIFWKKEKVI AHAQKYSKTY DKKTGDFKPG |
| 201 | TPWKTEFDAM AQKTLIKELL SKYAPLSIEL QKAILADNED |
| 241 | SNVNEVKRAK DVTPQEPENL SDLLSAPEEE QAKDVTPLED |
| 281 | DAQNSAADSV PDFVDPENGQ MDMLEGEDF |

A protein sequence for a RecT from a Collinsella stercoris phage (called CspRecT; NCBI: WP_006720782.1; SEQ ID NO: 19) is shown below.

| | |
|---|---|
| 1 | MNQIVKFTDD SGLAVQVTPD DVRRYICENA TEKEVGLFLQ |
| 41 | LCQTQRLNPF VKDAYLVKYG GAPASMITSY QVFNRRACRD |
| 81 | ANYDGIKSGV VVLRDGDVVH KRGAACYKKA GEELIGGWAE |
| 121 | VRFKDGRETA YAEVALDDYS TGKSNWAKMP GVMIEKCAKA |
| 161 | AAWRLAFPDT FQGMYAAEEM DQAQQPEQVR AQAEQPVDLQ |
| 201 | PIRELFKPYC EHFGITPAEG MTAVCGAVGA EGMHSMTEQQ |
| 241 | ARRARAWMEE EMAAPAVEAE YEVVDEGEVF |

The bacterial host cells can also have modified mismatch repair functions. For example, genes encoding mismatch repair enzymes can be modified to reduce mismatch repair. In some cases, one or more mismatch repair genes can be modified so that the encoded protein may bind to a mismatch site but be unable to correct the mismatch, resulting in unrepaired sites that are blocked from repair by other repair mechanisms.

One example of a gene involved in mismatch repair within E. coli is the mutL gene. A protein sequence for an Escherichia coli DSM 30083 MutL is shown below (NCBI ACZ50725.1; SEQ ID NO:20).

| | |
|---|---|
| 1 | MPIQVLPPQL ANQIAAGEVV ERPASVVKEL VENSLDAGAT |
| 41 | RIDIDIERGG AKLIRIRDNG CGIKKDELAL ALARHATSKI |
| 81 | ASLDDLEAII SIGFRGEALA SISSVSRLTL TSRTAEQQEA |
| 121 | WQAYAEGRDM NVTVKPAAHP VGTTLEVLDL FYNTPARRKF |
| 161 | LRTEKTEFNH IDEIIRRIAL ARFDVTINLS HNGKIVRQYR |
| 201 | AVPEGGQKER RLGAICGTAF LEQALAIEWQ HGDLTLRGWV |
| 241 | ADPNHTTPAL AEIQYCYVNG RMMRDRLINH AIRQACEDKL |
| 281 | GADQQPAFVL YLEIDPHQVD VNVHPAKHEV RFHQSRLVHD |
| 321 | FIYQGVLSVL QQQLETPLPL DDEPQPAPRS IPENRVAAGR |
| 361 | NHFAEPAARE PVAPRYTPAP ASGSRPAAPW PNAQPGYQKQ |
| 401 | QGEVYRQLLQ TPAPMQKLKA PEPQEPALAA NSQSEGRVLT |
| 441 | IVHSDCALLE RDGNISLLSL PVAERWLRQA QLTPGEAPVC |
| 481 | AQPLLIPLRL KVSAEEKSAL EKAQSALAEL GIDFQSDAQH |
| 521 | VTIRAVPLPL RQQNLQILIP ELIGYLAKQS VFEPGNIAQW |
| 561 | IARNIMSEHA QWSMAQAITL LADVERLCPQ LVKTPPGGLL |
| 601 | QSVDLHPAIK ALKDE |

A mutant E. coli mutL protein with a replacement of the glutamic acid (E) at position 32 with a lysine (K) is a dominant negative mutL (E32K) mutant protein, which even in the presence of wild type mutL, inhibits overall mismatch repair reaction, as well as MutH activation.

A nucleotide sequence for wild type Escherichia coli mutL is shown below (NCBI GU134327.1; SEQ ID NO:21).

| | | | | |
|---|---|---|---|---|
| 1 | ATGTAGCGAC | CAGTATGATC | AGTCAGTTGC | AACGCATTGG |
| 41 | CGAAATACAT | AAACGCCGAC | CAGAACACGC | GAGTCTCGGC |
| 81 | GTTCTGCGTT | CGCCGGATAT | CCCGTCAGTA | CTGGTCGAAA |
| 121 | CCGGTTTTAT | CAGCAACAAC | AGCGAAGAAC | GTTTGCTGGC |
| 161 | GAGCGACGAT | TACCAACAAC | AGCTGGCAGA | AGCCATTTAC |
| 201 | AAAGGCCTGC | GCAATTATTT | CCTTGCGCAT | CCGATGCAAT |
| 241 | CTGCGCCGCA | GGGGGCAACG | GCACAAACTG | CCAGTACGGT |
| 281 | GACGACGCCA | GATCGCACGC | TGCCAAACTA | AGGACGATTG |
| 321 | ATGCCAATTC | AGGTCTTACC | GCCACAACTG | GCGAACCAGA |
| 361 | TTGCCGCAGG | TGAGGTGGTC | GAGCGACCTG | CGTCGGTAGT |
| 401 | CAAAGAACTG | GTGGAAAACA | GCCTCGATGC | AGGTGCGACA |
| 441 | CGTATCGATA | TTGATATCGA | ACGCGGTGGG | GCGAAACTTA |
| 481 | TCCGCATTCG | TGATAACGGC | TGCGGTATCA | AAAAAGACGA |
| 521 | GCTGGCGCTG | GCGCTGGCGC | GTCATGCCAC | CAGTAAAATC |
| 561 | GCCTCTCTGG | ACGATCTCGA | AGCCATTATC | AGCCTGGGCT |
| 601 | TTCGCGGTGA | GGCGCTGGCG | AGTATCAGTT | CGGTTTCCCG |
| 641 | CCTGACGCTC | ACTTCACGCA | CCGCAGAACA | GCAGGAAGCC |
| 681 | TGGCAGGCCT | ATGCCGAAGG | GCGCGATATG | AACGTGACGG |
| 721 | TAAAACCGGC | GGCGCATCCT | GTGGGGACGA | CGCTGGAGGT |
| 761 | GCTGGATCTG | TTCTACAACA | CCCCGGCGCG | GCGCAAATTC |
| 801 | CTGCGCACCG | AGAAAACCGA | ATTTAACCAC | ATTGATGAGA |
| 841 | TCATCCGCCG | CATTGCGCTG | GCGCGTTTCG | ACGTCACGAT |
| 881 | CAACCTGTCG | CATAACGGTA | AAATTGTGCG | TCAGTACCGC |
| 921 | GCAGTGCCGG | AAGGCGGGCA | AAAAGAACGG | CGCTTAGGCG |
| 961 | CGATTTGCGG | CACCGCTTTT | CTTGAACAAG | CGCTGGCGAT |
| 1001 | TGAATGGCAA | CACGGCGATC | TCACGCTACG | CGGCTGGGTG |
| 1041 | GCCGATCCAA | ATCACACCAC | GCCCGCACTG | GCAGAAATTC |
| 1081 | AGTATTGCTA | CGTGAACGGT | CGCATGATGC | GCGATCGCCT |
| 1121 | GATCAATCAC | GCGATCCGCC | AGGCCTGCGA | AGACAAACTG |
| 1161 | GGGGCCGATC | AGCAACCGGC | ATTTGTGCTG | TATCTGGAGA |
| 1201 | TCGATCCGCA | TCAGGTGGAC | GTCAACGTGC | ACCCCGCCAA |
| 1241 | ACACGAAGTG | CGTTTCCATC | AGTCGCGTCT | GGTGCATGAT |
| 1281 | TTTATCTATC | AGGGCGTGCT | GAGCGTGCTA | CAACAGCAAC |
| 1321 | TGGAAACGCC | GCTACCGCTG | GACGATGAAC | CCCAACCTGC |
| 1361 | ACCGCGTTCC | ATTCCGGAAA | ACCGCGTGGC | GGCGGGGCGC |
| 1401 | AATCACTTTG | CAGAACCGGC | AGCTCGTGAG | CCGGTAGCTC |
| 1441 | CGCGCTACAC | TCCTGCGCCA | GCATCAGGCA | GTCGTCCGGC |
| 1481 | TGCCCCCTGG | CCGAATGCGC | AGCCAGGCTA | CCAGAAACAG |
| 1521 | CAAGGTGAAG | TGTATCGCCA | GCTTTTGCAA | ACGCCCGCGC |
| 1561 | CGATGCAAAA | ATTAAAAGCG | CCGGAACCGC | AGGAACCTGC |
| 1601 | ACTTGCGGCG | AACAGTCAGA | GTTTTGGTCG | GGTACTGACT |
| 1641 | ATCGTCCATT | CCGACTGTGC | GTTGCTGGAG | CGCGACGGCA |
| 1681 | ACATTTCACT | TTTATCCTTG | CCAGTGGCAG | AACGTTGGCT |
| 1721 | GCGTCAGGCA | CAATTGACGC | CGGGTGAAGC | GCCCGTTTGC |
| 1761 | GCCCAGCCGC | TGCTGATTCC | GTTGCGGCTA | AAAGTTTCTG |
| 1801 | CCGAAGAAAA | ATCGGCATTA | GAAAAAGCGC | AGTCTGCCCT |
| 1841 | GGCGGAATTG | GGTATTGATT | TCCAGTCAGA | TGCACAGCAT |
| 1881 | GTGACCATCA | GGGCAGTGCC | TTTACCCTTA | CGCCAACAAA |
| 1921 | ATTTACAAAT | CTTGATTCCT | GAACTGATAG | GCTACCTGGC |
| 1961 | GAAGCAGTCC | GTATTCGAAC | CTGGCAATAT | TGCGCAGTGG |
| 2001 | ATTGCACGAA | ATCTGATGAG | CGAACATGCG | CAGTGGTCAA |
| 2041 | TGGCACAGGC | CATAACCCTG | CTGGCGGACG | TGGAACGGTT |
| 2081 | ATGTCCGCAA | CTTGTGAAAA | CGCCGCCGGG | TGGTCTGTTA |
| 2121 | CAATCTGTTG | ATTTACATCC | GGCGATAAAA | GCCCTGAAAG |
| 2161 | ATGAGTGATA | TCAGTAAGGC | GAGCCTGCCT | AAGGCGATTT |
| 2201 | TTTTGATGGG | GCCGACGGCC | TCCGGTAAAA | CGGCGTTAGC |
| 2241 | CATTGAGCTG | CGTAAAATTT | TACCAGTAGA | GTTGATAAGC |
| 2281 | GTTGATTCTG | CCCTTATTTA | CAAAGGGATG | G |

An example of a mutL protein sequence from Mycobacterium tuberculosis is shown below (NCBI SGC95817.1; SEQ ID NO:22). A glutamic acid at position 32 is highlighted also below.

| | | | | |
|---|---|---|---|---|
| 1 | MAIRILPPQL | ANQIAAGEVV | ERPASVVKEL | VENSLDAGAT |
| 41 | RIDIDIERGG | EKLIRIRDNG | CGTAKDELAL | ALARHATSKI |
| 81 | ATLDDLEAIM | SMGERGEALA | SISSVSRLTF | TSRTRDQNEA |
| 121 | WQAYAEGREM | AVILKPAAHP | AGSTVEVLDL | FENTPARRKE |
| 161 | LRTEKTEFGH | IDEVVRRIAL | SREDVAINLT | HNGKLMRQYR |
| 201 | PVKAEDQQER | RLGAICGTAF | MQQALAVTWS | HEELEIRGWV |
| 241 | ASPAEYNGPA | DLQYCYVNGR | MMRDRLINHA | IRQAYEDRLS |
| 281 | GDQQPAYVLY | LTIDPRQVDV | NVHPAKHEVR | FHQARLVHDE |
| 321 | IYQAVLSVIK | QHEQPVSLFQ | PDPSAPQTQH | VPENRAAAGK |
| 361 | NIFEREEALT | PPPHRETTGG | GASHSGHSSG | KPAYSADKPV |
| 401 | YSPKEAGTYQ | SLMQTPAETR | PQLFPEKSTF | LSENRISAAS |
| 441 | EPVTEKSERV | SFGKILTLYP | PCYALIETDA | GTALFSLSKA |
| 481 | SHYLRCQQLI | PGEQGLKSQP | LMIPLQMALN | AQECETFTQF |
| 521 | AGVLRTFGTE | GSVSRGKATI | RTVSLPLRQQ | NLPHLIPELL |
| 561 | RFLADNPEGD | EKAIASRLAE | MLVSEPAAQS | KAQAVQLLAD |
| 601 | VERLCPQLVR | RPPADLLQLI | DLTEVVAALR | HE |

An example of a Salmonella enterica mutL protein sequence is shown below (NCBI ACL55048.1; SEQ ID NO:23). A glutamic acid at position 32 is highlighted also below.

```
    1 MPIQVLPPQL ANQIAAGEVV ERPASVVKEL VENSLDAGAT
  41 RVDIDIERGG AKLIRIRDNG CGTKKEELAL ALARHATSKI
  81 ASLDDLEAII SLGERGEALA SISSVSRLTL TSRTAEQAEA
121 WQAYAEGRDM DVTVKPAAHP VGTTLEVLDL FYNTPARRKF
161 MRTEKTEENH IDEIIRRIAL AREDVTLNLS HNGKLVRQYR
201 AVAKDGQKER RLGAICGTPF LEQALAIEWQ HGDLTLRGWV
241 ADPNHTTTAL TEIQYCHVNG RMMRDRLINH AIRQACEDKL
281 GADQQPAFVL YLEIDPHQVD VNVHPAKHEV RFHQSRLVHD
321 FIYQGVLSVL QQQTETTLPL EDIAPAPRHV PENRIAAGRN
361 HFAVPAEPTA AREPATPRYS GGASGGNGGR QSAGGWPHAQ
401 PGYQKQQGEV YRALLQTPTT SPAPEAVAPA LDGHSQSEGR
441 VLTIVCGDCA LLEHAGTIQL LSLPVAERWL RQAQLTPGQS
481 PVCAQPLLIP LRLKVSADEK AALQKAQSLL GELGTEFQSD
521 AQHVTIRAVP LPLRQQNLQI LIPELIGYLA QQTTFATVNI
561 AQWIARNVQS EHPQWSMAQA ISLLADVERL CPQLVKAPPG
601 GLLQPVDLHS AMNALKHE
```

An example of a Bacillus anthracis mutL protein sequence is shown below (NCBI WP_000516478.1; SEQ ID NO:24). A glutamic acid at a position homologous to position 32 (position 33) is also highlighted below.

| | | | | |
|---|---|---|---|---|
| 1 | MGKIRKLDDQ | LSNLIAAGEV | VERPASVVKE | LVENSIDANS |
| 41 | TSIEIHLEEA | GLSKIRIIDN | GDGTAEEDCI | VAFERHATSK |
| 81 | IKDENDLFRI | RTLGERGEAL | PSIASVSELE | LTTSTGDAPG |
| 121 | THLIIKGGDI | IKQEKTASRK | GTDTTVQNLF | FNTPARLKYM |
| 161 | KTIHTELGNI | TDIVYRIAMS | HPEVSLKLFH | NEKKLLHTSG |
| 201 | NGDVRQVLAS | IYSIQVAKKL | VPIEAESLDF | TIKGYVTLPE |
| 241 | VTRASRNYMS | TIVNGRYVRN | FVLMKAIQQG | YHTLLPVGRY |
| 281 | PIGFLSIEMD | PMLVDVNVHP | AKLEVRESKE | QELLKLIEET |
| 321 | LQAAFKKIQL | IPDAGVTTKK | KEKDESVQEQ | FQFEHAKPKE |
| 361 | PSMPEIVLPT | GMDEKQEEPQ | AVKQPTQLWQ | PSTKPIIEEP |
| 401 | IQEEKSWDSN | EEGFELEELE | EVREIKEIEM | NGNDLPPLYP |
| 441 | IGQMHGTYIF | AQNDKGLYMI | DQHAAQERIN | YEYFRDKVGR |
| 481 | VAQEVQELLV | PYRIDLSLTE | FLRVEEQLEE | LKKVGLFLEQ |
| 521 | FGHQSFIVRS | HPTWFPKGQE | TEIIDEMMEQ | VVKLKKVDIK |
| 561 | KLREEAAIMM | SCKASIKANQ | YLINDQIFAL | LEELRTTINP |
| 601 | YTCPHGRPIL | VHHSTYELEK | MFKRVM | |

An example of a Clostridium tetani mutL protein sequence is shown below (NCBI WP_011099529.1; SEQ ID NO:25). A glutamic acid at a position homologous to position 32 (position 33) is also highlighted below.

| | | | | |
|---|---|---|---|---|
| 1 | MKRINILDEC | TENKIAAGEV | VERPFSVVKE | LVENSIDAEA |
| 41 | KNTTVEVKNG | GQDLIKVSDD | GAGTYADDIQ | KAFLTHATSK |
| 81 | ILNIDDIFSL | NTMGFRGEAL | PSIASISKIL | LKSKPLSETS |
| 121 | GKEIYMEGGN | FISENDVGMN | TGTTIKVTDL | FYNVPARLKF |
| 161 | LKSSSRESSL | ISDIIQRLSL | ANPDIAFKLI | NNGKTVLNTY |
| 201 | GSGNLEDAIR | VIYGKKTLEN | ISYFESHSDI | ISVYGYIGNA |
| 241 | ELSRGSRNNQ | SIFVNKRYIK | SGLTTAAVEN | AFKSELTINK |
| 281 | FPFFVIFIDI | FPEYIDVNVH | PTKTEIKFKE | DKIVESFVEK |
| 321 | TVHESIKKSL | YKEFNEQIKE | DVKEDNKEII | KENPSLFQNV |
| 361 | EKVQIPIDLK | SASMDIERKS | LVNSVLCNEN | NIVKDNINKN |
| 401 | IYIDTKENLS | ENKLKNILKE | NTEDMVSKIP | DMKIIGQEDN |
| 441 | TYILAESVKN | LYIIDQHAAH | EKILFETYRD | KIKKDEVKSQ |
| 481 | LLLQPIVLEL | DSEDFSYYVD | NKELFYKTGF | NIEVEGENTI |
| 521 | NIREVPFIMG | KPDINNLFMD | IINNIKAMGS | GETIEVKYDS |
| 561 | IAMLACKSAV | KAHDKLSKEE | MEALINDLRF | AKDPENCPHG |
| 601 | RPTIIKTTSL | ELEKKEKRIQ | | |

An example of a Clostridium difficile mutL protein sequence is shown below (NCBI WP_211652115.1; SEQ ID NO:26). A glutamic acid at a position homologous to position 32 (position 34) is also highlighted below.

```
    1 MKNIINILDD LTINKIAAGE VVERPSSVVK ELIENSIDAG
  41 ANKISIDIID GGKSLIKTTD NGTGTPSSEV EKSFLRHATS
  81 KIKKIDDLYD LYSLGERGEA LASISAVSKL EMTIKTKDEI
121 IGTKIYVEGG KIISKEPIGS TNGTTIIIKD IFENTPARQK
161 FLKSTHAETI NISDLINKLA IGNPNIQFKY TNNNKQMLNT
201 PGDGKLVNTI RSIYGKETTE NIIDVEFKCN HFKMNGYIGN
241 NNIYRSNKNL QHIYINKRFV KSKIIIDATT ESYKSIIPIG
281 KHVVCFLNIE VDPSCIDVNI HPNKLEIKFE KEQEVYIELR
321 DFLKVKLIHS NLIGKYATYS DKKTQPRIAI NSREKSTDYK
361 LRNNDLLEST HKNSNTTKGK DEVIEVVTIS SEKPINEFQS
401 VSEVLNASVE DDVKNINYLS EDSVNDNIQE EFQVDGTKNE
441 GNYYLGDSIK DSEEEYSCSS KRKFSLYGYS VIGVVENTYI
481 ILSKDDSMYL LDQHAAHERI LYERYMEKFY RQDINMQILL
521 DPVVIEVSNV DMLQIENNLE LFMKFGFELE IFGNNHIMVR
561 CVPTIFGVPE TEKFILQIID NIEETTSNYD LKGERFASMA
601 CRSAIKANDK IYDIEIKSLL EQLEKCENPF TCPHGRPIMV
641 EISKTEIEKM FKRIM
```

An example of a Haemophilus influenzae mutL protein sequence is shown below (NCBI AVJ09575.1; SEQ ID NO:27). A glutamic acid at position 32 is also highlighted below.

| | | | | |
|---|---|---|---|---|
| 1 | MPIRILSPQL | ANQIAAGEVV | ERPASVVKEL | VENSLDAGAN |
| 41 | KIQIDIENGG | ANLIRIRDNG | CGTPKEELSL | ALARHATSKI |
| 81 | ADLDDLEAIL | SIGERGEALA | SISSVSRLTL | TSRTEEQTEA |
| 121 | WQVYAQGRDM | ETTIKPASHP | VGTTVEVANL | FENTPARRKF |
| 161 | LRTDKTEFAH | IDEVIRRIAL | TKENTAFTLT | HNGKIVRQYR |
| 201 | PAFDLNQQLK | RVAVICGDDF | VKNALRIDWK | HDDLHLSGWV |
| 241 | ATPNESRTQN | DLSYCYINGR | MVRDKVISHA | IRQAYAQYLP |
| 281 | TDAYPAFVLF | IDLNPHDVDV | NVHPTKHEVR | FHQQRLIHDE |
| 321 | IYEGTSYALN | NQEQLNWHTE | QSAVENHEEN | TVREPQPNYS |
| 361 | IRPNRAAAGQ | NSFAPQYHEK | PQQNQPHFSN | TPVLPNHVST |
| 401 | GYRDYRSDAP | SKTEQRLYAE | LLRTLPPTAQ | KDISNTAQQN |
| 441 | ISDTAKIIST | EIIECSSHLR | ALSLIENRAL | LLQQNQDFFL |
| 481 | LSLEKLQRLQ | WQLALQQIQI | EQQPLLIPIV | FRLTEAQFQA |
| 521 | WQQYSDNEKK | IGFEFIENQA | QLRLTLNKVP | NVLRTQNLQK |
| 561 | CVMAMLTRDE | NSSPFLTALC | AQLECKTEDA | LADALNLLSE |
| 601 | TERLLTQTNR | TAFTQLLKPV | NWQPLLDEI | |

An example of a Staphylococcus aureus mutL protein sequence is shown below (NCBI YP_499806.1; SEQ ID NO:28). A glutamic acid at a position homologous to position 32 (position 33) is also highlighted below.

| | | | | |
|---|---|---|---|---|
| 1 | MGKIKELQTS | LANKIAAGEV | VERPSSVVKE | LLENAIDAGA |
| 41 | TEISIEVEES | GVQSIRVVDN | GSGTEAEDLG | LVFHRHATSK |
| 81 | LDQDEDLFHI | RTLGERGEAL | ASISSVAKVT | LKTCTDNANG |
| 121 | NEIYVENGEI | LNHKPAKAKK | GTDILVESLF | YNTPARLKYI |
| 161 | KSLYTELGKI | TDIVNRMAMS | HPDIRIALIS | DGKTMLSING |
| 201 | SGRINEVMAE | IYGMKVARDL | VHISGDTSDY | HIEGFVAKPE |
| 241 | HSRSNKHYIS | IFINGRYIKN | FMLNKAILEG | YHTLLTIGRE |
| 281 | PICYINIEMD | PILVDVNVHP | TKLEVRISKE | EQLYQLIVSK |
| 321 | IQEAFKDRIL | IPKNNLDYVP | KKNKVLHSFE | QQKIEFEQRQ |
| 361 | NTENNQEKTF | SSEESNSKPF | MEENQNDEIV | IKEDSYNPFV |
| 401 | TKTSESLIAD | DESSGYNNTR | EKDEDYFKKQ | QEILQEMDQT |
| 441 | FDSNDGTTVQ | NYENKASDDY | YDVNDIKGTK | SKDPKRRIPY |
| 481 | MEIVGQVHGT | YIIAQNEFGM | YMIDQHAAQE | RIKYEYERDK |
| 521 | IGEVINEVQD | LLIPLTFHFS | KDEQLVIDQY | KNELQQVGTM |
| 561 | LEHFGGHDYI | VSSYPVWFPK | DEVEEIIKDM | IELILEEKKV |
| 601 | DIKKLREDVA | IMMSCKKSIK | ANHYLQKHEM | SDLIDQLREA |
| 641 | EDPFTCPHGR | PIIINFSKYE | LEKLFKRVM | |

An example of a Streptococcus pneumoniae mutL protein sequence is shown below (NCBI ABO44018.1; SEQ ID NO:29). A glutamic acid at a position homologous to position 32 (position 33) is also highlighted below.

| | | | | |
|---|---|---|---|---|
| 1 | MSHIIELPEM | LANQIAAGEV | IERPASVVKE | LVENAIDAGS |
| 41 | SQIIIEIEEA | GLKKVQTTDN | GHGTAHDEVE | LALRRHATSK |
| 81 | IKNQADLFRI | RTLGFRGEAL | PSIASVSVLT | LLTAVDGASH |
| 121 | GTKLVARGGE | VEEVIPATSP | VGTKVCVEDL | FENTPARLKY |
| 161 | MKSQQAELSH | IIDIVNRLGL | AHPEISFSLI | SDGKEMTRTA |
| 201 | GTGQLRQAIA | GTYGLASAKK | MIEIENSDLD | FEISGFVSLP |
| 241 | ELTRANRNYI | SLFINGRYIK | NFLLNRAILD | GFGSKLMVGR |
| 281 | FPLAVIHIHI | DPYLADVNVH | PTKQEVRISK | EKELMTLVSE |
| 321 | AIANSLKEQT | LIPDALENLA | KSTVRNREKV | EQTILPLKEN |
| 361 | TLYYEKTEPS | RPSQTEVADY | QVELTDEGQD | LTLFAKETLD |
| 401 | RLTKPAKLHE | AERKPANYDQ | LDHPELDLAS | IDKAYDKLER |
| 441 | EEASSFPELE | FFGQMHGTYL | FAQGRDGLYI | IDQHAAQERV |
| 481 | KYEEYRESIG | NVDQSQQQLL | VPYIFEFPAD | DALRLKERMP |
| 521 | LLEEVGVFLA | EYGENQFILR | EHPIWMAEEE | IESGTYEMCD |
| 561 | MLLLTKEVSI | KKYRAELAIM | MSCKRSIKAN | HRIDDHSARQ |
| 601 | LLYQLSQCDN | PYNCPHGRPV | LVHETKSDME | KMFRRIQENH |
| 641 | TSLRELGKY | | | |

A variety of single-stranded binding proteins (SSBs) can be expressed, either endogenously or recombinantly, to facilitate recombination during editing of the phages genomes. In general, the one or more single-stranded binding proteins so expressed are compatible with one or more single strand annealing proteins (SSAPs) to promote recombination during editing.

For example, one type of SSB that can be expressed by bacteria during editing of phage genomes is the Escherichia coli str. K-12 substr. MG1655 ssDNA-binding protein with the following sequence (NCBI NP_418483.1; SEQ ID NO:30).

| | | | | |
|---|---|---|---|---|
| 1 | MASRGVNKVI | LVGNLGQDPE | VRYMPNGGAV | ANTTLATSES |
| 41 | WRDKATGEMK | EQTEWHRVVL | FGKLAEVASE | YLRKGSQVYI |
| 81 | EGQLRTRKWT | DQSGQDRYTT | EVVVNVGGTM | QMLGGRQGGG |
| 121 | APAGGNIGGG | QPQGGWGQPQ | QPQGGNQFSG | GAQSRPQQSA |
| 161 | PAAPSNEPPM | DFDDDIPF | | |

A nucleotide sequence for the above Escherichia coli str. K-12 substr. MG1655 ssDNA-binding protein is shown below (NCBI NC_000913.3; SEQ ID NO:31)

| | | | | |
|---|---|---|---|---|
| 1 | ATGGCCAGCA | GAGGCGTAAA | CAAGGTTATT | CTCGTTGGTA |
| 41 | ATCTGGGTCA | GGACCCGGAA | GTACGCTACA | TGCCAAATGG |
| 81 | TGGCGCAGTT | GCCAACATTA | CGCTGGCTAC | TTCCGAATCC |
| 121 | TGGCGTGATA | AAGCGACCGG | CGAGATGAAA | GAACAGACTG |
| 161 | AATGGCACCG | CGTTGTGCTG | TTCGGCAAAC | TGGCAGAAGT |
| 201 | GGCGAGCGAA | TATCTGCGTA | AAGGTTCTCA | GGTTTATATC |
| 241 | GAAGGTCAGC | TGCGTACCCG | TAAATGGACC | GATCAATCCG |
| 281 | GTCAGGATCG | CTACACCACA | GAAGTCGTGG | TGAACGTTGG |
| 321 | CGGCACCATG | CAGATGCTGG | GTGGTCGTCA | GGGTGGTGGC |
| 361 | GCTCCGGCAG | GTGGCAATAT | CGGTGGTGGT | CAGCCGCAGG |
| 401 | GCGGTTGGGG | TCAGCCTCAG | CAGCCGCAGG | GTGGCAATCA |
| 441 | GTTCAGCGGC | GGCGCGCAGT | CTCGCCCGCA | GCAGTCCGCT |
| 481 | CCGGCAGCGC | CGTCTAACGA | GCCGCCGATG | GACTTTGATG |
| 521 | ATGACATTCC | GTTCTGA | | |

Another example of an Escherichia coli SSB protein sequence is shown below (NCBI WP_222563270.1; SEQ ID NO:32).

```
    1 MWKRGENKVI LMGRAGKDAE VRYTPNGTAI ASLTLATEIS
  41 YNDNEGKEQK ETEWHDIVIF GKKAEAAGKY FKKGMMLYFV
  81 GRIRNNKWQG TDGKMRSNKE IVIDNNGEMQ MLPGAGPRNT
121 SAEGQGGTEN HDEPPFPDMN DYPQ
```

An example of a Klebsiella pneumoniae SSB protein sequence is shown below (NCBI ANI75733.1; SEQ ID NO:33).

```
    1 MINIKYMRCI MWKRGENKVI LMGRSGKDAE LRYTPNGTAI
  41 ASLTLATEIS YSDNEGKEQK ETEWHDIVIF GKKAEAAGKY
  81 FKKGMMLYFV GRIRNNKWQG TDGKMRSNKE IVIDNNGEMQ
121 MLPGAGPRNT SAEGQGGTEN HDEPPFPDMN DYPQ
```

Another example of a Klebsiella pneumoniae SSB protein sequence is shown below (NCBI WP_102017779.1; SEQ ID NO:34).

```
    1 MWKRGENKVI LMGRAGKDAE VRYTPNGTAI ASLTLATEIS
  41 YSDNEGKEQK ETEWHDIVIF GKKARAAGKY FKKGMMLYFV
  81 GRIRNNKWQG TDGKMRSNKE IVIDNNGEMQ MLPGAGPRNT
121 SAEGQGGFEN HDEPPFPDMN DYPQ
```

An example of a multi-species Enterobacterales SSB protein sequence is shown below (NCBI WP_011091064.1; SEQ ID NO:35).

| | | | | |
|---|---|---|---|---|
| 1 | MWKRGENKVI | LMGRAGKDAE | VRYTPNGTAI | ASLTLATEIS |
| 41 | YSDNEGKEQK | ETEWHDIVIF | GKKAEAAGKY | FKKGMMLYFV |
| 81 | GRIRNNKWQG | TDGKMRSNKE | IVIDNNGEMQ | MLPGAGPRNT |
| 121 | SAEGLGGTEN | HDEPPFPDMN | DYPQ | |

Another example of a multi-species Enterobacterales SSB protein sequence is shown below (NCBI WP_004187334.1; SEQ ID NO:36).

```
    1 MWKRGENKVI LMGRAGKDAE VRYTPNGTAI ASLTLATEIS
  41 YSDNEGKEQK ETEWHDIVIF GKKAEAAGKY FKKGMMLYFV
  81 GRIRNNKWQG TDGKMRSNKE IVIDNNGEMQ MLPGAGPRNT
121 SAEGQGGIEN HDEPPFPDMN DYPQ
```

An example of a Salmonella enterica SSB protein sequence is shown below (NCBI EEC4048472.1; SEQ ID NO:37).

```
    1 MWKRGENKVI LMGRAGKDAE VRYTPNGTAI ASLTLATEIS
  41 YSDNEGKEQK ETEWHDIVIF GKKAEAAGKY FKKGMMLYFV
  81 GRIRNNKWQG TDGKMRSNKE IVIDNNGEMQ MLPGAGPRNT
121 STEGLGGTEN HDEPPFPDMN DYPQ
```

Another example of a Salmonella enterica SSB protein sequence is shown below (NCBI EGJ1037365.1; SEQ ID NO:38).

```
    1 MWKRGENKVI LMGRAGKDAE VRYTPNGTAI ASLTLATEIS
  41 YSDNEGKEQK ETEWHDIVIF GKKAEAAGKY FKKGMMLYFV
  81 GRIRNNKWQG TDGKMRSNKE IVIDNNGEMQ MLPGAGPRNT
121 SAEGHGGIEN HDEPPFPDMN DYPQ
```

An example of an Enterobacter hormaechei subsp. Steigerwaltii SSB protein sequence is shown below (NCBI HAS0717021.1; SEQ ID NO:39).

```
    1 KRGENKVILM GRAGKDAEVR YTPNRTAIAS LTLATEISYS
  41 DNEGKEQKET EWHDIVIFGK KAEAAGKYFK KGMMLYFVGR
  81 IRNNKWQGTD GKMRSNKEIV IDNNGEMQML PGAGPRNTSA
121 EGQGGIENHD EPPEPDMNDY PQ
```

An example of a Citrobacter freundii SSB protein sequence is shown below (NCBI EHL7056105.1; SEQ ID NO:40).

```
    1 VILMGRSGKD AELRYTPNGT AIASLTLATE ISYSDNEGKE
  41 QKETEWHDIV IFGKKAEAAG KYFKKGMMLY FVGRIRNNKW
  81 QGTDGKMRSN KEIVIDNNGE MQMLPGAGPR NTSAEGQGGI
121 ENHDEPPFPD MNDYPQ
```

Variants and homologs of any of the sequences described here can also be used in the methods and systems described herein. For example, such variants and homologs can have less than 100% sequence identity to any of the sequences described herein. The variants and homologs can have about at least 40% sequence identity, or at least 50% sequence identity, or at least 60% sequence identity, or at least 70% sequence identity, or at least 80% sequence identity, or at least 90% sequence identity, or at least 95% sequence identity, or at least 96% sequence identity, or at least 97% sequence identity, or at least 98% sequence identity, or at least 99% sequence identity, or 60-99% sequence identity, or 70-99% sequence identity, or 80-99% sequence identity, or 90-95% sequence identity, or 90-99% sequence identity, or 95-97% sequence identity, or 97-99% sequence identity, or 100% sequence identity with any of sequences described herein.

### Bacteriophages

Various types of bacteriophages (phages) can be modified using the editing components, bacterial host cells, and methods described herein. Bacteriophages are ubiquitous viruses, found wherever bacteria exist. It is estimated there are more than 1031 bacteriophages on the planet, more than every other organism on Earth, including bacteria. Many types of bacteriophages can be modified by the methods and editing systems described herein. However, in some cases the phages to be modified are DNA phages. For example, the phages to be modified can have double-stranded genomes.

The phage with the genomes to be edited can be lytic phages, which are easier to isolate than temperate phages. However, in some cases the phages with the genomes that will be edited can be temperate phages. For example, one type of editing that can be performed using the methods described herein can be converting temperate phages or lysogenic phages into lytic phages.

The vast majority of phages belong to the order of Caudovirales, which are tailed phages that have dsDNA and an isometric capsid. Caudovirales is comprised of three phylogenetically-related families that are discriminated by tail morphology: Myoviridae (long contractile tails), Siphoviridae (long non-contractile tails), and Podoviridae (short tails) (Ackermann, 2007; Krupovic, Prangishvili, Hendrix, & Bamford, 2011). The most well-studied tailed phages are the coliphages λ (Siphoviridae), T4 (Myoviridae), and T7 (Podoviridae) which infect Escherichia coli. Any such phage species can be genomically modified using the methods described herein. The bacteriophage database at the website phagesdb.org provides information and sequences for bacteriophages that can used to identify target sites for editing. The NCBI database also provides sequences for bacteriophages that can used to identify target sites for editing.

Examples of bacteriophages that can be modified include:
bacteriophage lambda, T2, T5, T7, PDX, vB_EcoS-2862I, vB_EcoS-2862II, vB_EcoS-2862III, vB_EcoS-2862IV, vB_EcoS-2862V, vB_EcoS-260201, vB_EcoS-26020II, vB_EcoS-26020III, vB_EcoS-26020IV, vB_EcoS-26020V;
bacteriophage Pal (ATCC 12,175-B1), Pa2 (ATCC 14203-B1), and Pa11 (ATCC 14205-B1) that can inhibit P. aeruginosa strain PAO1;
bacteriophage ϕMR11 that can lyse multidrug resistant S. aureus;
bacteriophage KP DP1, SA DP1, PA DP4, and EC DP3 (isolated from wastewater against multi-drug resistant bacteria including K. pneumoniae, S. aureus, P. aeruginosa, and E. coli);
bacteriophage AB-Navy1, AB-Navy2, AB-Navy3, and AB-Navy4, which can inhibit the wound infection caused by multi-drug resistant Acinetobacter baumannii;
bacteriophage Sb-1, MR-5 and MR-10 that can inhibit or lyse Staphylococcus aureus;
bacteriophage Kpn1, Kpn2, Kpn3, Kpn4, Kpn5, KØ1, KØ2, KØ3, KØ4, KØ5 that can inhibit Klebsiella pneumoniae.

Some pathogenic bacterial toxins are encoded by bacteriophage genomes such that the host bacteria are only pathogenic when lysogenized by the toxin-encoding phage. Examples of toxins that can be encoded by bacteriophages are cholera toxin in Vibrio cholerae, diphtheria toxin in Corynebacterium diphtheriae, botulinum neurotoxin in Clostridium botulinum, the binary toxin of Clostridium difficile, and Shiga toxin of Shigella species. Without their phage-encoded toxins, these bacterial species are either much less pathogenic or not pathogenic at all. Hence, toxin-encoding genes in bacteriophage genomes can be deleted or knocked out using the methods described herein before those phage are further modified.

Bacterial cells can have a couple of mechanisms that can interfere with phage infection, including receptor/adsorption blocking; abortive infection; clustered, regularly interspaced short palindromic repeats (CRISPR) with CRISPR-associated (Cas) proteins (CRISPR-Cas); and restriction modification (RM). Phage can be modified to make them less vulnerable to these bacterial cell defense mechanisms.

CRISPRs (Clustered Regularly Interspaced Short Palindromic Repeats), also known as SPIDRs (SPacer Interspersed Direct Repeats), constitute a family of DNA loci that are usually specific to a particular bacterial species. CRISPR loci generally include a distinct class of interspersed short sequence repeats (SSRs) that were recognized by specific bacterial proteins (Ishino et al, J. BacterioL, 169:5429-5433 (1987); and Nakata et al., J. BacterioL, 171:3553-3556 (1989)). Similar interspersed SSRs have been identified in Haloferax mediterranei, Streptococcus pyogenes, Anabaena, and Mycobacterium tuberculosis (See, Groenen et al., Mol. Microbiol., 10:1057-1065 (1993); Hoe et al., Emerg. Infect. Dis., 5:254-263 (1999); Masepohl et al, Biochim. Biophys. Acta 1307:26-30 (1996); and Mojica et al, Mol. Microbiol, 17:85-93 (1995)). The CRISPR loci typically differ from other SSRs by the structure of the repeats, which have been termed short regularly spaced repeats (SRSRs) (Janssen et al, OMICS J. Integ. Biol., 6:23-33 (2002); and Mojica et al, Mol. Microbiol., 36:244-246 (2000)). In general, the repeats are short elements that occur in clusters that are regularly spaced by unique intervening sequences with a substantially constant length (Mojica et al., (2000), supra). Although the repeat sequences are highly conserved between strains, the number of interspersed repeats and the sequences of the spacer regions typically differ from strain to strain (van Embden et al., J. Bacteriol., 182:2393-2401 (2000)). CRISPR loci have been identified in more than 40 prokaryotes (See e.g., Jansen et al, Mol. Microbiol., 43:1565-1575 (2002); and Mojica et al, (2005)) including, but not limited to Aeropyrum, Pyrobaculum, Sulfolobus, Archaeoglobus, Halocarcula, Methanobacteriumn, Methanococcus, Methanosarcina, Methanopyrus, Pyrococcus, Picrophilus, Thernioplasnia, Corynebacterium, Mycobacterium, Streptomyces, Aquifrx, Porphvromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

In general, “a CRISPR system” refers collectively to transcripts and other elements involved in the expression of or directing the activity of CRISPR-associated (“Cas”) genes, including sequences encoding a Cas gene, and a CRISPR array nucleic acid sequence including a leader sequence and at least one repeat sequence. A CRISPR system can be a type I, type II, or type III CRISPR system.

In some embodiments, the bacterial cells express a CRISPR enzyme such as one or more of the following Cas proteins: Cas1, Cas1B, Cas2, Cas3, Cas4, Cas5, Cas6, Cas7, Cas8, Cas9 (also known as Csn1 and Csx12), Cas1O, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, homologs thereof, or modified versions thereof.

Cas1 and Cas2 are found in type I, type II, or type III CRISPR systems, and they are involved in spacer acquisition. In the I-E system of E. coli, Cas1 and Cas2 form a complex where a Cas2 dimer bridges two Cas1 dimers. In this complex, Cas2 performs a non-enzymatic scaffolding role, binding double-stranded fragments of invading (phage) DNA, while Cas1 binds the single-stranded flanks of the DNA and catalyzes their integration into CRISPR arrays.

Protospacers that are adjacent to short (3-5 bp) DNA sequences termed protospacer adjacent motifs (PAM). The PAMs are important for type I and type II systems during acquisition. In type I and type II systems, protospacers are excised at positions adjacent to a PAM sequence, with the other end of the spacer is cut using a ruler mechanism, thus maintaining the regularity of the spacer size in the CRISPR array. The conservation of the PAM sequence differs between CRISPR-Cas systems and may be evolutionarily linked to Cas1 and the leader sequence. For example, one type protospacer adjacent motif (PAM) sequence has “AAG” sequence.

For example, some bacterial cells have a Class II CRISPR system where endoribonucleases (cas nucleases) are expressed that can preferentially cleave specific sequences, including certain repeat sequences in DNA, various U-rich regions in RNAs, sites near a protospacer adjacent motif (PAM). Class II CRISPR systems, for example, can include a cluster of four genes Cas9, Cas1, Cas2, and Csn1, that employ a tracrRNA and a crispr RNA (crRNA). In this system, targeted DNA double-strand break (DSB) may be generated in four sequential steps. First, the pre-crRNA and tracrRNA, may be expressed. Second, tracrRNA may hybridize to the direct repeats of pre-CRISPR guide RNA (pre-crRNA), which is then processed into mature crRNAs containing individual spacer sequences. Third, the mature crRNA:tracrRNA complex can direct a cas nuclease to the DNA target consisting of the protospacer and the corresponding PAM sequence via heteroduplex formation between the spacer region of the crRNA and the protospacer DNA. The cas nuclease may then cleave target DNA upstream of the PAM site to create a double-stranded break within the protospacer. Such cleavage can undermine or destroy a phage.

However, Cas nucleases bind to nucleic acids only in presence of a specific sequence, called protospacer adjacent motif (PAM), on the non-targeted DNA strand. Therefore, the locations in the genome that can be targeted by different Cas proteins are limited by the locations of these PAM sequences. The cas nuclease cuts 3-4 nucleotides upstream of the PAM sequence. Hence, one method to generate phage that are not vulnerable to CRISPR-based bacterial defense mechanisms is to modify PAM sites in phage genomes so that cas nucleases cannot bind to their genomic DNA.

TABLE 1
Examples of Cas nucleases and
their PAM sequences.

| CRISPR | Organism | PAM Sequence |
|---|---|---|
| Nucleases | Isolated From | (5′ to 3′) |
| SpCas9 | Streptococcus | NGG |
| | pyogenes | |
| SaCas9 | Staphylococcus | NGRRT or NGRRN |
| | aureus | |
| NmeCas9 | Neisseria | NNNNGATT |
| | meningitidis | |
| CjCas9 | Campylobacter | NNNNRYAC |
| | jejuni | |
| StCas9 | Streptococcus | NNAGAAW |
| | thermophilus | |
| LbCpf1 | Lachnospiraceae | TTTV |
| (Cas12a) | bacterium | |
| AsCpf1 | Acidaminococcus | TTTV |
| (Cas12a) | sp. | |
| AacCas12b | Alicyclobacillus | TTN |
| | acidiphilus | |
| BhCas12b v4 | Bacillus hisashii | ATTN, TTTN and GTTN |
| Cas14 | Uncultivated archea | T-rich PAM |
| | | sequences, e.g. |
| | | TTTA for dsDNA |
| | | cleavage, no PAM |
| | | sequence |
| | | requirement |
| | | for ssDNA |
| Cas3 | in silico analysis | No PAM sequence |
| | of various | requirement |
| | prokaryotic genomes | |

In Table 1, an “N” in a PAM sequence means that any nucleotide is present; an R means that an A or a G is present; a W means that an A or a T is present; a Y means that a T or a C is present; and a V means that an A, C or G is present.

### Retrons

One or more modified retron nucleic acids can be used for genomic editing of phage. The retron nucleic acids employed can include one or more types of modified retrons, modified ncRNAs, modified reverse transcribed noRNAs, or libraries of such modified retron nucleic acids. The retron nucleic acids are modified to include exogenous or heterologous nucleic acids, thereby allowing production in vivo of substantial amounts of templates for genomic repair, templates for reverse transcriptases, and the like.

Retrons in nature generally include two elements, one that encodes a reverse transcriptase and a second that is single-stranded DNA/RNA hybrid. Wild type retrons are about 2 kb long. They contain a single operon controlling the synthesis of an RNA transcript carrying three loci, msr, msd, and ret. The DNA portion of a retron is encoded by the msd gene, the RNA portion is encoded by the mar gene, while the product of the ret gene is a reverse transcriptase. The retron msr RNA is a non-coding RNA (ncRNA) produced by retron elements and is the immediate precursor to the synthesis of msDNA.

While msDNA and reverse transcribed DNA (RT-DNA) are related, the term reverse transcribed DNA (RT-DNA) is used herein to refer to any retron-related reverse transcribed DNA, whether modified or not, while the term msDNA refers to wild type, natural, or unmodified retron msDNA.

The ncRNA of naturally occurring retrons includes a pre-msr sequence, an msr gene encoding multicopy single-stranded RNA (msRNA). The msd gene encodes a multicopy single-stranded DNA (msDNA), the post-msd sequence, and a ret gene encoding a reverse transcriptase. Synthesis of DNA by the retron-encoded reverse transcriptase provides a DNA/RNA chimeric product which is composed of single-stranded DNA encoded by the msd gene linked to single-stranded RNA encoded by the msr gene. The retron msr RNA contains a conserved guanosine residue at the end of a stem loop structure. A strand of the msr RNA is joined to the 5′ end of the msd single-stranded DNA by a 2′-5′ phosphodiester linkage at the 2′ position of this conserved guanosine residue.

For example, a wild type retron-Eco1 ncRNA (also called ec86 or retron-Eco1 ncRNA) can have the sequence shown below as SEQ ID NO:41.

| | | | | |
|---|---|---|---|---|
| 1 | TGCGCACCCT | TAGCGAGAGG | TTTATCATTA | AGGTCAACCT |
| 41 | CTGGATGTTG | TTTCGGCATC | CTGCATTGAA | TCTGAGTTAC |
| 81 | TGTCTGTTTT | CCTTGTTGGA | ACGGAGAGCA | TCGCCTGATG |
| 121 | CTCTCCGAGC | CAACCAGGAA | ACCCGTTTTT | TCTGACGTAA |
| 161 | GGGTGCGCA | | | |

An example of an Eco1 human-codon optimized reverse transcriptase (RT) sequence that can be used is shown below as SEQ ID NO:42.

| | | | | |
|---|---|---|---|---|
| 1 | ATGAAATCTG | CAGAGTATCT | GAATACGTTC | CGCCTTAGGA |
| 41 | ATTTGGGCCT | CCCCGTGATG | AACAATCTCC | ACGATATGAG |
| 81 | CAAGGCGACT | CGAATATCCG | TGGAAACGCT | GAGACTGCTC |
| 121 | ATCTATACAG | CAGACTTTCG | GTACAGGATC | TACACGGTCG |
| 161 | AAAAGAAGGG | GCCTGAGAAA | CGCATGCGAA | CAATTTATCA |
| 201 | ACCTAGCCGA | GAGCTCAAGG | CGTTGCAGGG | CTGGGTTCTT |
| 241 | CGAAACATCC | TTGACAAACT | CTCATCATCA | CCCTTTAGTA |
| 281 | TTGGGTTTGA | AAAGCACCAA | AGCATCCTTA | ACAACGCGAC |
| 321 | GCCACACATA | GGTGCCAATT | TCATATTGAA | CATCGACTTG |
| 361 | GAGGATTTTT | TTCCGAGCCT | CACAGCCAAT | AAAGTGTTCG |
| 401 | GTGTTTTTCA | CAGTCTTGGG | TACAATCGCC | TTATTAGTTC |
| 411 | CGTTCTTACC | AAGATTTGTT | GTTACAAGAA | TCTCTTGCCC |
| 481 | CAGGGAGCAC | CCAGCAGTCC | GAAATTGGCG | AATTTGATTT |
| 521 | GTTCCAAGCT | CGATTATCGA | ATACAAGGGT | ACGCGGGCAG |
| 561 | CCGGGGACTC | ATCTATACCC | GCTACGCAGA | CGATCTTACG |
| 601 | CTGTCTGCCC | AATCAATGAA | GAAGGTCGTA | AAGGCGCGGG |
| 641 | ATTTCTTGTT | TTCTATCATC | CCGTCCGAGG | GCTTGGTAAT |
| 681 | TAATTCCAAA | AAGACTTGTA | TCTCAGGACC | ACGATCTCAG |
| 721 | CGAAAAGTGA | CAGGACTCGT | CATTTCTCAA | GAAAAAGTCG |
| 761 | GTATAGGGAG | AGAGAAGTAT | AAGGAAATCC | GCGCGAAGAT |
| 801 | CCACCACATA | TTCTGTGGCA | AGAGCAGCGA | GATAGAACAC |
| 841 | GTCCGAGGCT | GGTTGTCCTT | CATACTGAGC | GTGGACTCAA |
| 881 | AAAGCCACCG | CCGGTTGATC | ACCTATATTT | CAAAACTGGA |
| 921 | AAAGAAATAT | GGAAAGAACC | CACTCAACAA | AGCTAAAACA |
| 961 | TAG | | | |

An example of an Eco2 human-codon optimized reverse transcriptase (RT) sequence is shown below as SEQ ID NO:43.

| | | | | |
|---|---|---|---|---|
| 1 | ATGACAAAAA | CTTCAAAGCT | GGATGCGCTG | CGGGCGGCTA |
| 41 | CTAGTAGGGA | AGATTTGGCG | AAGATTCTCG | ACATAAAGTT |
| 81 | GGTGTTTCTG | ACAAACGTGT | TGTACCGCAT | AGGATCCGAC |
| 121 | AACCAGTATA | CGCAATTCAC | AATACCCAAA | AAGGGTAAAG |
| 161 | GTGTCCGCAC | CATCAGCGCA | CCAACGGACC | GACTTAAGGA |
| 201 | TATACAGAGG | AGGATTTGTG | ATCTTCTTAG | TGACTGTAGG |
| 241 | GATGAAATCT | TTGCGATTAG | GAAGATCTCT | AATAATTACT |
| 281 | CATTCGGCTT | CGAAAGAGGA | AAATCAATTA | TACTCAATGC |
| 321 | TTACAAGCAT | CGAGGGAAGC | AAATTATATT | GAACATCGAC |
| 361 | CTTAAGGACT | TCTTTGAGAG | CTTTAACTTT | GGGAGAGTCC |
| 401 | GGGGGTACTT | TCTCTCCAAC | CAGGACTTCT | TGTTGAACCC |
| 441 | AGTTGTGGCA | ACAACGTTGG | CGAAGGCCGC | CTGCTACAAC |
| 481 | GGGACTCTGC | CTCAGGGGTC | CCCATGTTCC | CCTATTATAA |
| 521 | GTAACCTTAT | CTGTAACATT | ATGGACATGC | GGCTCGCAAA |
| 561 | GCTCGCCAAG | AAGTACGGCT | GCACTTATAG | TCGATATGCG |
| 601 | GATGACATTA | CGATCAGCAC | CAATAAAAAT | ACCTTCCCGT |
| 641 | TGGAGATGGC | GACTGTGCAG | CCTGAAGGGG | TTGTGCTGGG |
| 681 | CAAAGTGCTC | GTAAAGGAGA | TTGAAAATTC | AGGTTTCGAG |
| 721 | ATTAACGATT | CTAAGACTAG | ATTGACCTAC | AAAACAAGTA |
| 761 | GGCAAGAAGT | CACCGGGCTG | ACGGTTAATC | GGATTGTAAA |
| 801 | CATTGATCGG | TGCTACTACA | AAAAGACGAG | GGCGCTGGCT |
| 841 | CACGCATTGT | ATCGGACAGG | AGAATATAAG | GTCCCAGACG |
| 881 | AGAACGGTGT | TCTGGTATCT | GGAGGGCTTG | ACAAGTTGGA |
| 921 | GGGTATGTTT | GGGTTTATCG | ACCAGGTGGA | TAAATTCAAC |
| 961 | AACATTAAAA | AAAAGTTGAA | TAAGCAACCC | GACAGATATG |
| 1001 | TTCTGACAAA | TGCCACTTTG | CACGGATTTA | AGCTCAAATT |
| 1041 | GAACGCCAGG | GAGAAAGCCT | ATAGCAAATT | CATCTACTAC |
| 1081 | AAATTCTTCC | ACGGTAATAC | TTGTCCCACG | ATCATAACAG |
| 1121 | AGGGTAAGAC | GGATAGGATT | TACCTTAAAG | CTGCCCTCCA |
| 1161 | TAGCCTCGAG | ACAAGTTATC | CTGAACTGTT | TCGGGAGAAA |
| 1201 | ACAGATAGTA | AGAAGAAGGA | GATAAATCTG | AATATTTTTA |
| 1241 | AAAGCAATGA | GAAGACCAAG | TATTTCCTGG | ATCTCAGCGG |
| 1281 | CGGCACAGCA | GACCTCAAGA | AATTCGTGGA | ACGCTACAAA |
| 1321 | AATAACTACG | CTTCCTATTA | CGGCAGCGTA | CCGAAACAAC |
| 1361 | CGGTGATAAT | GGTGCTTGAT | AACGACACAG | GCCCGTCAGA |
| 1401 | CCTGTTGAAC | TTTTTGAGAA | ACAAAGTTAA | GAGTTGTCCA |
| 1441 | GATGATGTAA | CAGAAATGCG | CAAGATGAAG | TACATACATG |
| 1481 | TGTTTTACAA | TCTGTACATA | GTTCTGACTC | CCCTGTCTCC |
| 1521 | ATCTGGAGAG | CAAACGTCTA | TGGAGGACCT | CTTTCCTAAA |
| 1561 | GATATATTGG | ACATTAAGAT | AGATGGCAAG | AAATTCAATA |
| 1601 | AAAACAATGA | CGGTGACTCC | AAAACAGAGT | ATGGGAAGCA |
| 1001 | CATATTCTCA | ATGCGCGTTG | TACGAGATAA | AAAGAGGAAG |
| 1001 | ATAGATTTCA | AGGCATTTTG | CTGTATCTTC | GATGCTATTA |
| 1001 | AGGATATTAA | AGAACATTAC | AAACTGATGT | TGAATTCCTA |
| 1001 | G | | | |

An example of an Eco1 wild-type retron reverse transcriptase sequence is shown below as SEQ ID NO:44.

| | | | | |
|---|---|---|---|---|
| 1 | KSAEYLNTER | LRNLGLPVMN | NLHDMSKATR | ISVETLRLLI |
| 41 | YTADFRYRIY | TVEKKGPEKR | MRTIYQPSRE | LKALQGWVLR |
| 81 | NILDKLSSSP | FSIGFEKHQS | ILNNATPHIG | ANFILNIDLE |
| 121 | DFFPSLTANK | VEGVEHSLGY | NRLISSVLTK | ICCYKNLLPQ |
| 161 | GAPSSPKLAN | LICSKLDYRI | QGYAGSRGLI | YTRYADDLTL |
| 201 | SAQSMKKVVK | ARDELFSIIP | SEGLVINSKK | TCISGPRSQR |
| 241 | KVTGLVISQE | KVGIGREKYK | EIRAKIHHIF | CGKSSEIEHV |
| 281 | RGWISFILSV | DSKSHRRLIT | YISKLEKKYG | KNPLNKAKT |

An example of an Eco2 wild-type retron reverse transcriptase sequence is shown below as SEQ ID NO:45.

| | | | | |
|---|---|---|---|---|
| 1 | MTKTSKLDAL | RAATSREDLA | KILDIKLVFL | INVLYRIGSD |
| 41 | NQYTQFTIPK | KGKGVRTISA | PTDRLKDIQR | RICDLLSDCR |
| 81 | DEIFAIRKIS | NNYSEGFERG | KSIILNAYKH | RGKQIILNID |
| 121 | LKDFFESENF | GRVRGYFLSN | QDFLLNPVVA | TTLAKAACYN |
| 161 | GTLPQGSPCS | PIISNLICNI | MDMRLAKLAK | KYGCTYSRYA |
| 201 | DDITISTNKN | TEPLEMATVQ | PEGVVIGKVL | VKEIENSGFE |
| 241 | INDSKTRITY | KTSRQEVTGL | TVNRIVNIDR | CYYKKTRALA |
| 281 | HALYRTGEYK | VPDENGVLVS | GGLDKLEGMF | GFIDQVDKEN |
| 321 | NIKKKLNKQP | DRYVLTNATL | HGFKLKLNAR | EKAYSKFIYY |
| 361 | KFFHGNTCPT | IITEGKTDRI | YLKAALHSLE | TSYPELFREK |
| 401 | TDSKKKEINL | NIEKSNEKTK | YFLDLSGGTA | DLKKEVERYK |
| 441 | NNYASYYGSV | PKQPVIMVLD | NDTGPSDLLN | FLRNKVKSCP |
| 481 | DDVTEMRKMK | YIHVFYNLYI | VLTPLSPSGE | QTSMEDLEPK |
| 521 | DILDIKIDGK | KENKNNDGDS | KTEYGKHIFS | MRVVRDKKRK |
| 561 | IDFKAFCCIF | DAIKDIKEHY | KLMLNS | |

An example of a sequence for an Eco4 retron reverse transcriptase is shown below as SEQ ID NO:46.

| | | | | |
|---|---|---|---|---|
| 1 | MSIDIETTLQ | KAYPDFDVLL | KSRPATHYKV | YKIPKRTIGY |
| 41 | RIIAQPTPRV | KAIQRDIIEI | LKQHTHIHDA | ATAYVDGKNI |
| 81 | LDNAKIHQSS | VYLLKLDLVN | FENKITPELL | FKALARQKVD |
| 121 | ISDINKNLLK | QFCFWNRTKR | KNGALVLSVG | APSSPFISNI |
| 161 | VMSSFDEEIS | SECKENKISY | SRYADDLTFS | TNERDVLGLA |
| 201 | HQKVKTTLIR | FEGTRIIINN | NKIVYSSKAH | NRHVTGVTLT |
| 241 | NNNKLSLGRE | RKRYITSLVF | KFKEGKLSNV | DINHLRGLIG |
| 281 | FAYNIEPAFI | ERLEKKYGES | TIKSIKKYSE | GG |

An example of a sequence for a Sen2 retron reverse transcriptase is shown below as SEQ ID NO:47.

| | | | | |
|---|---|---|---|---|
| 1 | MDILQHISDL | LLTKKSEIIS | FSLTAPYRYK | IYKIAKRNSD |
| 41 | KKRTIAHPSK | ELKFIQREIT | EYLTDKLPVH | ECAFAYKKGS |
| 81 | SIKTNAQVHL | HTKYLLKMDE | ENFFPSITPR | LFFSKLRLAN |
| 121 | IDLTADDKVL | LENILFFKSK | RNSNLRLSIG | APSSPLISNE |
| 161 | VMYFWDIEVQ | EICSKIGVNY | TRYADDLTFS | TNNKDVLFDI |
| 201 | PDMLENVLPK | YSLGRIRINH | EKTVESSKGH | NRHVTGITLT |
| 241 | NDNKLSIGRE | RKRKISAMIH | HFINGKLSTD | ECNKLVGLLA |
| 281 | FAKNIEPSFY | KSMVIKYGSD | NIYKLQKQKD | K |

Variants and homologs of any of the sequences described here can also be used in the methods and systems described herein. For example, such variants and homologs can have less than 100% sequence identity to any of the sequences described herein. The variants and homologs can have about at least 40% sequence identity, or at least 50% sequence identity, or at least 60% sequence identity, or at least 70% sequence identity, or at least 80% sequence identity, or at least 90% sequence identity, or at least 95% sequence identity, or at least 96% sequence identity, or at least 97% sequence identity, or at least 98% sequence identity, or at least 99% sequence identity, or 60-99% sequence identity, or 70-99% sequence identity, or 80-99% sequence identity, or 90-95% sequence identity, or 90-99% sequence identity, or 95-97% sequence identity, or 97-99% sequence identity, or 100% sequence identity with any of sequences described herein.

Other types of retrons are described throughout the application and can be used in the methods described herein.

Modified (e.g., engineered) retrons can have alterations in different locations relative to the corresponding wild type retrons. However, not every modification provides a stable retron, stable retron nucleic acids, or retron ncRNAs that can yield good amounts of reverse transcribed DNA.

One example of a location for modification of retron nucleic acids is within a self-complementary region (stem region, which has sequence complementarity to the pre-msr sequence), wherein the length of the self-complementary region can be lengthened relative to the corresponding region of a native retron. Complementarity between the strands of the stem region is maintained when adding nucleotides but the length of the stem region can be increased. Such modifications result in an engineered retron that provides enhanced production of RT-DNA.

In certain embodiments, a complementary region of a retron ncRNA or RT-DNA has a length at least 1, at least 2, at least 4, at least 6, at least 8, at least 10, at least 12, at least 14, at least 16, at least 18, at least 20, at least 30, at least 40, or at least 50 nucleotides longer than the wild-type self-complementary region. For example, the self-complementary region may have a length ranging from at least 1 to at least 50 nucleotides longer than the native or wild-type complementary region, including any length within this range, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 34, 35, 36, 37, 38, 39, 40, 41, 42, 43, 44, 45, 46, 47, 48, 49, 50 or more nucleotides longer. Such modifications should retain the complementarity of the stem structure. In certain embodiments, the self-complementary region has a length ranging from 1 to 16 nucleotides longer than the wild-type complementary region.

To create more abundant RT-DNA, for example, the ncRNA SEQ ID NO:48 sequence shown below, with the native self-complementary 3′ and 5′ ends highlighted in bold (at positions 1-12 and 158-169), can be extended at positions 1 and 169 to extend the self-complementary region.

| | | | | |
|---|---|---|---|---|
| 1 | TGCGCACCCT | TAGCGAGAGG | TTTATCATTA | AGGTCAACCT |
| 41 | CTGGATGTTG | TTTCGGCATC | CTGCATTGAA | TCTGAGTTAC |
| 81 | TGTCTGTTTT | CCTTGTTGGA | ACGGAGAGCA | TCGCCTGATG |
| 121 | CTCTCCGAGC | CAACCAGGAA | ACCCGTTTTT | TCTGACGTAA |
| 161 | GGGTGCGCA | | | |

For example, the following engineered “ncRNA extended” (SEQ ID NO:49) construct is shown below, where the additional nucleotides that extend the self-complementary region are shown in italics and with underlining.

| | | | | |
|---|---|---|---|---|
| 1 | TGATAAGATT | CCGTATGCGC | ACCCTTAGCG | AGAGGTTTAT |
| 41 | CATTAAGGTC | AACCTCTGGA | TGTTGTTTCG | GCATCCTGCA |
| 81 | TTGAATCTGA | GTTACTGTCT | GTTTTCCTTG | TTGGAACGGA |
| 121 | GAGCATCGCC | TGATGCTCTC | CGAGCCAACC | AGGAAACCCG |
| 161 | TTTTTTCTGA | CGTAAGGGTG | CGCATACGGA | ATCTTATCA |

In some cases, the additional nucleotides can be added to any position in the self-complementary region, for example, anywhere within positions 1-12 and 158-169 of the SEQ ID NO:48 or SEQ ID NO:49 sequence.

In certain embodiments, sequences of the msr gene, msd gene, and ret gene used in the engineered retron may be derived from any bacterial retron operon. Representative retrons are available such as those from gram-negative bacteria including, without limitation, any of the Eco1, Eco2, Eco3, Eco4 retrons; myxobacteria retrons such as Myxococcus xanthus retrons (e.g., Mx65, Mx162); Stigmatella aurantiaca retrons (e.g., Sa163); Escherichia coli retrons (e.g., Ec48, E67, Ec73, Ec78, EC83, EC86, EC107, and Ec107); Salmonella enterica; Vibrio cholerae retrons (e.g., Vc81, Vc95, Vc137); Vibrio parahaemolyticus (e.g., Vc96); and Nannocystis exedens retrons (e.g., Ne144). Retron msr gene, msd gene, and ret gene nucleic acid sequences as well as retron reverse transcriptase protein sequences may be derived from any source. Representative retron sequences, including msr gene, msd gene, and ret gene nucleic acid sequences and reverse transcriptase protein sequences are listed in the National Center for Biotechnology Information (NCBI) database. See, for example, NCBI entries: Accession Nos. EF428983, M55249, EU250030, X60206, X62583, AB299445, AB436696, AB436695, M86352, M30609, M24392, AF427793, AQ3354, and AB079134; all of which sequences (as entered by the date of filing of this application) are herein incorporated by reference in their entireties.

The retron ncRNAs can be modified to enhance production of retron reverse transcribed DNA in a host cell or to provide host cells with genomic editing components or other useful proteins and/or nucleic acids. Any of the foregoing retron sequences (or variants thereof) can include variant or mutant nucleotides, added nucleotides, or fewer nucleotides.

For example, a parental ncRNA can be modified by addition of nucleotides to a stem or loop as described herein. Before modification the parental ncRNA can have at least about 80-100% sequence identity to any region of the retrons described herein, including any percent identity within this range, including any percent identity within this range, such as 81, 82, 83, 84, 85, 86, 87, 88, 89, 90, 91, 92, 93, 94, 95, 96, 97, 98, or 99% sequence identity to any region of the retron sequences described herein (including those defined by accession number). Such parental retrons can be used to construct an engineered retron or vector system comprising an engineered retron, as described herein.

The modified retron nucleic acids can include exogenous or heterologous nucleotides or nucleic acid segments. For example, the exogenous or heterologous nucleotide or nucleic acid segments can add at least 1, at least 2, at least 4, at least 6, at least 8, at least 10, at least 12, at least 14, at least 16, at least 18, at least 20, at least 30, at least 40, at least 50, at least 60, at least 70, at least 80, at least 90, at least 100, at least 125, at least 150, at least 175, or at least 200 nucleotides to parental retron nucleic acids, to thereby generate modified retron nucleic acids.

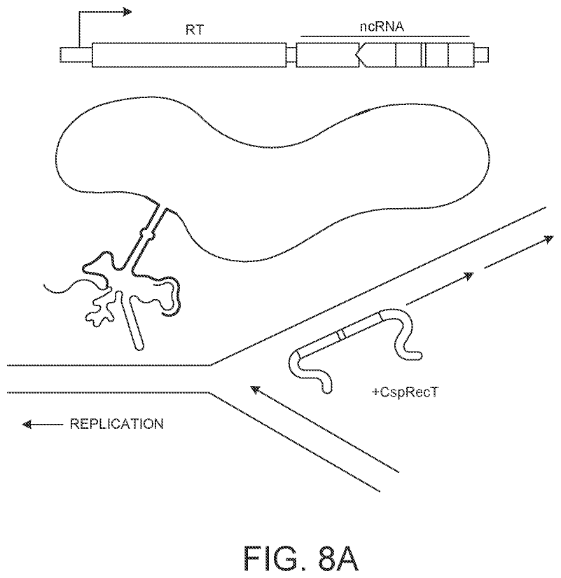

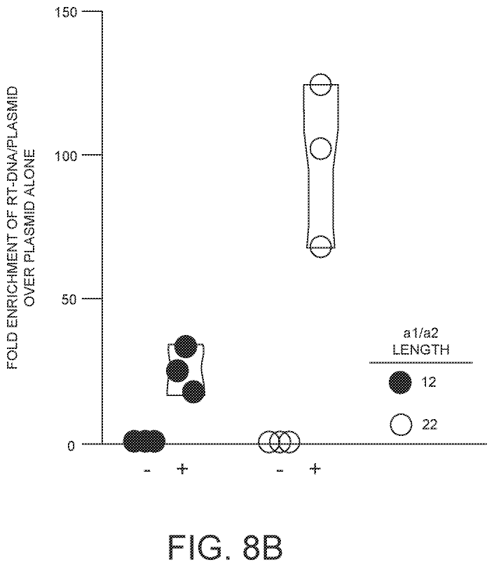

One example of a locus for insertion of exogenous or heterologous nucleotide or nucleic acid segments into retron nucleic acids is a loop portion of a stem-loop (see, e.g., FIG. 8A).

As described above, the retron nucleic acids can be modified with respect to the native retron to include one or more heterologous sequences of interest, including an ncRNA template for a donor DNA suitable for use in gene editing (e.g., by insertion during phage replication), and in some cases a barcode. Such heterologous sequences may be inserted, for example, into the ncRNA coding region in the expression cassette. Upon transcription, the noRNA will include an RNA segment encoding the donor DNA. The ncRNA can be partially reverse transcribed to generate the donor RNA.

In some cases, the donor DNA sequence of interest can be inserted into the loop of the msd stem loop of the retron.

In some cases, engineered retron nucleic acids can include unique barcodes to facilitate detection and analysis of engineered sites. Barcodes may comprise one or more nucleotide sequences that are used to identify a nucleic acid or cell with which the barcode is associated. Such barcodes may be inserted for example, into the loop region of the msd-encoded DNA. Barcodes can be 3-1000 or more nucleotides in length, preferably 10-250 nucleotides in length, and more preferably 10-30 nucleotides in length, including any length within these ranges, such as 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, or 1000 nucleotides in length. A barcode may be used to identify the presence of a particular genetically modified site within a phage. The use of barcodes allows retrons from different cells to be pooled in a single reaction mixture for sequencing while still being able to trace a particular retron, ncRNA, donor DNA, reverse transcriptase, or cas nuclease back to the colony from which it originated.

Therefore, expression cassettes with segments encoding any of the ncRNAs, donor DNAs, and/or reverse transcriptases, and/or other proteins that can facilitate editing can be linked to a barcode that is inserted into a genome and can be recovered by sequencing. In this way, many variables can be identified and evaluated in the same population of phage to assess relative integration frequency.

The modified retron constructs can have a non-native configuration with non-native spacing between the ncRNA coding region and the reverse transcriptase (ret) coding region. For example, it can be useful to separate the expression cassettes that include the ncRNA coding region and the reverse transcriptase (ret) coding region. Hence, the ncRNA and the reverse transcriptase may be separated in a trans arrangement rather than provided in the natural cis arrangement. In some embodiments, the ret gene is provided in a trans arrangement that eliminates a cryptic stop signal for the reverse transcriptase, which allows the generation of longer single stranded DNAs from the engineered retron construct.

Amplification of retron nucleic acids may be performed, for example, before introduction into cells, before ligation into vectors, or at other times. Any method for amplifying the retron constructs may be used, including, but not limited to polymerase chain reaction (PCR), isothermal amplification, nucleic acid sequence-based amplification (NASBA), transcription mediated amplification (TMA), strand displacement amplification (SDA), and ligase chain reaction (LCR). In one embodiment, the retron constructs comprise common 5′ and 3′ priming sites to allow amplification of retron sequences in parallel with a set of universal primers. In another embodiment, a set of selective primers is used to selectively amplify a subset of retron sequences from a pooled mixture.

The methods and compositions therefore allow production of modified phage as well as design of improved components for editing phage genomes.

### Libraries

Libraries of modified nucleic acids (modified ncRNAs, modified DNAs encoding ncRNAs, modified DNAs encoding reverse transcriptases, and combinations thereof) can be used in the expression cassettes, constructs and methods described herein. Thousands of nucleic acids encoding different reverse transcriptases, editing proteins (SSAP, SSBs, mutant repair proteins, etc.) and combinations thereof can be synthesized and used to optimize genomic editing of phage.

A golden-gate-based cloning strategy (Engler et al., PLOS One (Nov. 5, 2008)) can be used to clone such nucleic acids, and then large pools of different reverse transcriptases, different donor DNAs, editing proteins (SSAPs, SSBs, mutant repair proteins, etc.) and combinations thereof can be expressed in multiplexed vectors.

For example, a plasmid having or encoding a parental ncRNA nucleic acid insert (e.g., one that encodes a donor DNA) can be subjected to directed mutagenesis to generate a population of plasmids with different nucleic acid inserts that encode the differently modified ncRNAs (providing a multitude of donor DNA templates). The plasmid can be an expression vector (or an expression cassette) so that the nucleic acid inserts can be expressed to generate the different modified retron ncRNAs, along with the one or more reverse transcriptases, editing proteins (SSAP, SSBs, mutant repair proteins, etc.), and combinations thereof.

Alternatively, a population of oligonucleotides encoding ncRNAs (e.g., one that encodes a donor DNA) can be subjected to directed mutagenesis to generate a population of variant oligonucleotides, which can be inserted into expression vectors or expression cassettes so that the oligonucleotide inserts can be expressed to generate the variant ncRNAs, that can provide the donor DNAs. Genomic editing results that generate a population of potentially edited phages in host cells expressing a reverse transcriptase can be evaluated by sequencing of the phage genomes.

### Expression Systems

Modified and unmodified single strand annealing proteins (SSAPs), single-stranded DNA binding proteins (SSBs), mismatch repair (e.g., mutL) mutants, reverse transcriptases, retrons, retron nucleic acids, ncRNAs, retron constructs, or combinations thereof can be incorporated into and expressed from one or more expression cassettes or expression vectors. Such expression cassettes or expression vectors can be manipulated/constructed in vitro (or in vivo) and then separately or jointly introduced to bacterial cells.

A “vector” is a composition of matter that can be used to deliver a nucleic acid of interest to the interior of a cell. Nucleic acids encoding modified and/or unmodified single strand annealing proteins (SSAPs, e.g., RecT recombinases), single-stranded DNA binding proteins (SSBs), mismatch repair (e.g., mutL) mutants, reverse transcriptases, retrons, retron nucleic acids, ncRNAs, retron constructs, or combinations thereof can be introduced into a cell via a single vector or via multiple separate vectors to allow expression of the single strand annealing proteins (SSAPs), single-stranded DNA binding proteins (SSBs), mismatch repair (e.g., mutL) mutants, reverse transcriptases, retrons, retron nucleic acids, ncRNAs, retron constructs, or combinations thereof in host cells.

Vectors typically include control elements operably linked to the retron sequences, which allow for expression in vivo in the host cells. For example, the segment encoding the single strand annealing proteins (SSAPs), single-stranded DNA binding proteins (SSBs), mismatch repair (e.g., mutL) mutants, reverse transcriptases, retrons, retron nucleic acids, ncRNAs, retron constructs, or combinations thereof can be operably linked to the same or different promoters to allow expression thereof.

In some embodiments, heterologous sequences encoding desired products of interest (e.g., donor polynucleotides for gene editing, barcodes, or combinations thereof) may be inserted in the segment encoding the ncRNA.

In some embodiments, the engineered retron nucleic acids for editing phage can be provided by one or more vectors. For example, the ncRNA and the reverse transcriptase may be provided by the same vector (i.e., cis arrangement of such retron elements), wherein the vector comprises a promoter operably linked to the segment encoding the ncRNA and the segment encoding the reverse transcriptase. In some embodiments, a second promoter is operably linked to the segment encoding the reverse transcriptase. Alternatively, the segment encoding the reverse transcriptase may be incorporated into a second vector that does not include the ncRNA, msr gene or the msd gene (i.e., trans arrangement).

Similarly, nucleic acids encoding single strand annealing proteins (SSAPs, e.g., RecT recombinases), single-stranded DNA binding proteins (SSBs), mismatch repair (e.g., mutL) mutants, or combinations thereof can be expressed from one or more expression cassettes or expression vectors.

Numerous vectors are available including, but not limited to, linear polynucleotides, polynucleotides associated with ionic or amphiphilic compounds, plasmids, and viruses. Thus, the term “vector” includes an autonomously replicating plasmid. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In certain embodiments, the nucleic acid comprising one or more wild type or modified sequences is under transcriptional control of a promoter. A “promoter” refers to a DNA sequence recognized by the synthetic machinery of the cell, or introduced synthetic machinery, required to initiate the specific transcription of a gene. The term promoter will be used here to refer to a group of transcriptional control modules that are clustered around the initiation site for RNA polymerase. Such promoters can be obtained from commercially available plasmids, using techniques available in the art. See, e.g., Sambrook et al., supra. Enhancer elements may be used in association with the promoter to increase expression levels of the constructs.

Expression vectors for expressing one or more products or nucleic acids can include a promoter “operably linked” to a nucleic acid segment encoding the product of interest, ncRNA or and/or the reverse transcriptase. The phrase “operably linked” or “under transcriptional control” as used herein means that the promoter is in the correct location and orientation in relation to a polynucleotide to control the initiation of transcription by RNA polymerase and expression of the product, noRNA and/or the reverse transcriptase.

Typically, transcription terminator/polyadenylation signals will also be present in the expression construct.

Alternatively, a polynucleotide encoding a viral 2A-self cleaving peptide can be used to allow production of multiple protein products (e.g., Cas9, bacteriophage recombination proteins, retron reverse transcriptase) from a single vector. One or more 2A linker peptides can be inserted between the coding sequences in the multicistronic construct. The 2A peptide, which is self-cleaving, allows co-expressed proteins from the multicistronic construct to be produced at equimolar levels. 2A peptides from various viruses may be used, including, but not limited to 2A peptides derived from the foot-and-mouth disease virus, equine rhinitis A virus, Thosea asigna virus and porcine teschovirus-1. See, e.g., Kim et al. (2011) PLOS One 6 (4): e18556, Trichas et al. (2008) BMC Biol. 6:40, Provost et al. (2007) Genesis 45 (10): 625-629, Furler et al. (2001) Gene Ther. 8 (11): 864-873; herein incorporated by reference in their entireties.

In certain embodiments, the expression construct comprises a plasmid sequence suitable for transforming a bacterial host. Numerous bacterial expression vectors are available. Bacterial expression vectors include, but are not limited to, pACYC177, pASK75, pBAD, pBADM, pBAT, pCal, pET, pETM, pGAT, pGEX, pHAT, pKK223, pMal, pProEx, pQE, and pZA31. Bacterial plasmids may contain antibiotic selection markers (e.g., ampicillin, kanamycin, erythromycin, carbenicillin, streptomycin, or tetracycline resistance), a lacZ gene (β-galactosidase produces blue pigment from x-gal substrate), fluorescent markers (e.g., GFP. mCherry), or other markers for selection of transformed bacteria. See, e.g., Sambrook et al., supra.

In order to effect expression of single strand annealing proteins (SSAPS), single-stranded DNA binding proteins (SSBs), mismatch repair (e.g., mutL) mutants, reverse transcriptases, retrons, retron nucleic acids, ncRNAs, retron constructs, or combinations thereof, one or more expression constructs can be delivered into a cell. This delivery may be accomplished in vitro, as in laboratory procedures for transforming host cell lines.

Several methods for the transfer of expression constructs into cultured cells are contemplated. These include the use of calcium phosphate precipitation, DEAE-dextran, electroporation, direct microinjection, DNA-loaded liposomes, lipofectamine-DNA complexes, cell sonication, gene bombardment using high velocity microprojectiles, and receptor-mediated transfection (see, e.g., Graham and Van Der Eb (1973) Virology 52:456-467; Chen and Okayama (1987) Mol. Cell Biol. 7:2745-2752; Rippe et al. (1990) Mol. Cell Biol. 10:689-695; Gopal (1985) Mol. Cell Biol. 5:1188-1190; Tur-Kaspa et al. (1986) Mol. Cell. Biol. 6:716-718; Potter et al. (1984) Proc. Natl. Acad. Sci. USA 81:7161-7165); Harland and Weintraub (1985) J. Cell Biol. 101:1094-1099); Nicolau & Sene (1982) Biochim. Biophys. Acta 721:185-190; Fraley et al. (1979) Proc. Natl. Acad. Sci. USA 76:3348-3352; Fechheimer et al. (1987) Proc Natl. Acad. Sci. USA 84:8463-8467; Yang et al. (1990) Proc. Natl. Acad. Sci. USA 87:9568-9572; Wu and Wu (1987) J. Biol. Chem. 262:4429-4432; Wu and Wu (1988) Biochemistry 27:887-892; herein incorporated by reference). Some of these techniques may be successfully adapted for use.

Delivery of constructs encoding the single strand annealing proteins (SSAPs), single-stranded DNA binding proteins (SSBs), mismatch repair (e.g., mutL) mutants, reverse transcriptases, retrons, retron nucleic acids, ncRNAs, or combinations thereof to a cell can be accomplished with or without vectors.

A variety of methods for introducing nucleic acids into a host cell are available. Commonly used methods include chemically induced transformation, typically using divalent cations (e.g., CaCl₂)), dextran-mediated transfection, polybrene mediated transfection, lipofectamine and LT-1 mediated transfection, electroporation, protoplast fusion, encapsulation of nucleic acids in liposomes, and direct microinjection of the nucleic acids comprising engineered retrons into nuclei. See, e.g., Sambrook et al. (2001) Molecular Cloning, a laboratory manual, 3rd edition, Cold Spring Harbor Laboratories, New York, Davis et al. (1995) Basic Methods in Molecular Biology, 2^{nd} edition, McGraw-Hill, and Chu et al. (1981) Gene 13:197; herein incorporated by reference in their entireties.

In certain embodiments, the vector or cassette encoding the single strand annealing proteins (SSAPs), single-stranded DNA binding proteins (SSBs), mismatch repair (e.g., mutL) mutants, reverse transcriptases, retrons, retron nucleic acids, ncRNAs, or combinations thereof may be stably integrated into the genome of the cell. This integration may be in the cognate location and orientation, or it may be integrated in a random, non-specific location (gene augmentation). In yet further embodiments, the expression vector or cassette may be stably maintained in the cell as a separate, episomal segment of DNA. Such nucleic acid segments or “episomes” encode sequences sufficient to permit maintenance and replication independent of or in synchronization with the host cell cycle. In some cases, how the vector or cassette comprising the nucleic acids encoding single strand annealing proteins (SSAPs), single-stranded DNA binding proteins (SSBs), mismatch repair (e.g., mutL) mutants, reverse transcriptases, retrons, retron nucleic acids, or combinations thereof are delivered to a cell and where in the cell the nucleic acid remains is dependent on the type of expression construct employed.

In yet another embodiment, the expression construct may simply consist of naked recombinant DNA or plasmids comprising the retron nucleic acids (e.g., expression cassettes). Transfer of the constructs may be performed by any of the methods mentioned above which physically or chemically permeabilize the cell membrane. This is particularly applicable for transfer in vitro but it may be applied to in vivo use as well.

In other cases, a naked DNA expression construct may be transferred into cells by particle bombardment. This method depends on the ability to accelerate DNA-coated microprojectiles to a high velocity allowing them to pierce cell membranes and enter cells without killing them (Klein et al. (1987) Nature 327:70-73). Several devices for accelerating small particles have been developed. One such device relies on a high voltage discharge to generate an electrical current, which in turn provides the motive force (Yang et al. (1990) Proc. Natl. Acad. Sci. USA 87:9568-9572). The microprojectiles may consist of biologically inert substances, such as tungsten or gold beads.

In a further embodiment, the expression construct may be delivered using liposomes. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh & Bachhawat (1991) Liver Diseases, Targeted Diagnosis and Therapy Using Specific Receptors and Ligands, Wu et al. (Eds.), Marcel Dekker, NY, 87-104). Also contemplated is the use of lipofectamine-DNA complexes.

In some cases, a construct encoding single strand annealing proteins (SSAPs, e.g., RecT recombinases), single-stranded DNA binding proteins (SSBs), mismatch repair (e.g., mutL) mutants, reverse transcriptases, retrons, retron nucleic acids, ncRNAs, or combinations thereof may be contacted with host cells in combination with a cationic lipid. Examples of cationic lipids include, but are not limited to, lipofectin, DOTMA, DOPE, and DOTAP. The publication of WO/0071096, which is specifically incorporated by reference, describes different formulations, such as a DOTAP:cholesterol or cholesterol derivative formulation that can effectively be used for gene therapy. Other disclosures also discuss different lipid or liposomal formulations including nanoparticles and methods of administration; these include, but are not limited to, U.S. Patent Publication 20030203865, 20020150626, 20030032615, and 20040048787, which are specifically incorporated by reference to the extent they disclose formulations and other related aspects of delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

### Genomic Editing

Bacteriophages, which naturally shape bacterial communities, can be co-opted as a biological technology to help eliminate pathogenic bacteria from our bodies and food supply¹. Phage genome editing is a critical tool to engineer more effective phage technologies. However, editing phage genomes has traditionally been a low efficiency process that requires laborious screening, counter selection, or in vitro construction of modified genomes². These requirements impose limitations on the type and throughput of phage modifications, which in turn limit our knowledge and potential for innovation. Provided herein is a scalable approach for engineering phage genomes using recombitrons: modified bacterial retrons³ that generate recombineering donor DNA along with single stranded binding and annealing proteins to integrate those donors into phage genomes. This system can efficiently create genome modifications in multiple distinct phages without the need for counterselection. Moreover, the process is continuous, with edits accumulating in the phage genome the longer the phage is cultured with the host, and multiplexable, with different editing hosts contributing distinct mutations along the genome of a phage in a mixed culture. In lambda phage, as an example, recombitrons yield single-base substitutions at up to 99% efficiency, short (<20 base pair) insertions and deletions at 5-50%, and up to 5 distinct mutations installed on a single phage genome, all without counterselection and only a few hours of hands-on time.

The compositions and methods described herein provide genomic editing of phage genomes by supplying donor DNA and by using endogenously or recombinantly expressed proteins that facilitate transfer of the edited sequences from the donor DNA into the phage genomes during phage replication.

One problem with currently available phage editing methods is that donor DNA is not provided in sufficient amounts to the host cells to provide efficient phage genomic editing. The methods and compositions described herein solve this problem by expressing multiple copies of retron noncoding RNAs (ncRNAs) from an expression cassette or expression vector as templates for donor DNAs. Multiple copies of the donor DNAs are then generated from each ncRNA by reverse transcription.

Editing of phage genomes generally is done during phage replication. Once a bacteriophage attaches to a susceptible host, it pursues one of two replication strategies: lytic or lysogenic. During a lytic replication cycle, a phage attaches to a susceptible host bacterium, introduces its genome into the host cell cytoplasm, and utilizes the ribosomes of the host to manufacture its proteins. The host cell resources are rapidly converted to phage genomes and capsid proteins, which assemble into multiple copies of the original phage. As the host cell dies, it is either actively or passively lysed, releasing the new bacteriophage to infect another host cell. In the lysogenic replication cycle, the phage also attaches to a susceptible host bacterium and introduces its genome into the host cell cytoplasm. However, the phage genome is instead integrated into the bacterial cell chromosome or maintained as an episomal element where, in both cases, it is replicated and passed on to daughter bacterial cells without killing them. The phage with the genomes that will be edited can be lytic, temperate, or lysogenic phage. For example, one type editing that can be performed using the methods described here can be converting temperate or lysogenic phages into lytic phages.

In some cases, the donor DNA includes a sequence having a sequence identity of about 50%, 55%, 60%, 65%, 70%, 75%, 80%, 85%, 90%, 95%, 96%, 97%, 98% or 99% to a target phage genomic DNA sequence (or a complement thereof). In some cases, the donor DNA, or complement thereof, includes a sequence having a sequence identity of at least about 90%, 95%, 96%, 97%, 98%, or 99% to a target nucleic acid.

The target phage sequences can be any site in the phage genome. However, in some cases the donor DNA do not edit target phage sequences involved in phage cellular entry or phage replication. Instead, the donor DNA can, for example, target phage sequences that bacterial cells defensively target. In other words, the target sites in phage genomes are selected to improve phage killing or increase phage inhibition of bacterial growth.

The endogenously or recombinantly expressed proteins facilitate transfer of the editing sequences from the donor DNA into the phage genomes during phage replication. These proteins can include one or more single strand annealing proteins (SSAPs), single-stranded DNA binding proteins (SSBs), mutant mismatch repair proteins, or a combination thereof.

The methods described herein can therefore perform genomic editing without using clustered regularly interspaced short palindromic repeats (CRISPR)/CRISPR-associated (Cas) systems (see e.g., Marraffini and Sontheimer. Nature Reviews Genetics 11:181-190 (2010); Sorek et al. Nature Reviews Microbiology 2008 6:181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12:177-186).

### Definitions

The term “about” as used herein when referring to a measurable value such as an amount, a length, and the like, is meant to encompass variations of ±20% or ±10%, more preferably ±5%, even more preferably ±1%, and still more preferably ±0.1% from the specified value.

“Recombinant” as used herein to describe a nucleic acid molecule means a polynucleotide of retron, genomic, cDNA, bacterial, semisynthetic, or synthetic origin which, by virtue of its origin or manipulation, is not associated with all or a portion of the polynucleotide with which it is associated in nature.

The term “recombinant” as used with respect to a protein or polypeptide means a polypeptide produced by expression of a recombinant polynucleotide. In general, the polynucleotide of interest is cloned and then expressed in transformed organisms, for example, as described herein. The host organism expresses the foreign nucleic acids to produce the RNA, RT-DNA, or protein under expression conditions.

As used herein, a “cell” refers to any prokaryotic cell such as a bacteria. The methods described herein can be performed, for example, on a sample comprising a single cell or a population of cells. The term also includes genetically modified cells.

The term “transformation” refers to the insertion of an exogenous polynucleotide (e.g., an engineered retron) into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells,” “host cells”, “cells”, “cell lines”, “cell cultures”, and other such terms refer to cells which can be, or have been, used as recipients for recombinant vector or other transferred DNA, and include the original progeny of the original cell which has been transfected.

A “coding sequence” or a sequence which “encodes” a selected polypeptide or a selected RNA, is a nucleic acid molecule which is transcribed (in the case of DNA templates) into RNA and/or translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, single strand annealing proteins (SSAPs), single-stranded DNA binding proteins (SSBs), mismatch repair (e.g., mutL) mutants, reverse transcriptases, retrons, retron nucleic acids, or combinations thereof. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of an encoded protein or nucleic acid when the proper polymerases are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide or RNA sequence. For example, the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. The RNA sequence or a portion thereof contains a nucleotide sequence of at least 3 to 5 nucleotides, more preferably at least 8 to 10 nucleotides, and even more preferably at least 15 to 20 nucleotides.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein, DNA, or RNA or cause other adverse consequences. That is, a nucleic acid or peptide of this invention is purified if it is substantially free of cellular material, viral material, or culture medium when obtained from nature or when produced by recombinant DNA techniques, or free from chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

“Substantially purified” generally refers to isolation of a substance (nucleic acid, compound, polynucleotide, protein, polypeptide, peptide composition) such that the substance comprises the majority percent of the sample in which it resides. Typically, in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

“Purified polynucleotide” refers to a polynucleotide of interest or fragment thereof which is essentially free, e.g., contains less than about 50%, preferably less than about 70%, and more preferably less than about at least 90%, of the protein and/or nucleic acids with which the polynucleotide is naturally associated. Techniques for purifying polynucleotides of interest are available in the art and include, for example, disruption of the cell containing the polynucleotide with a chaotropic agent and separation of the polynucleotide(s) and proteins by ion-exchange chromatography, affinity chromatography and sedimentation according to density.

The term “transfection” is used to refer to the uptake of foreign DNA by a cell. A cell has been “transfected” when exogenous DNA has been introduced inside the cell membrane. A number of transfection techniques are generally available. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material and includes uptake of peptide-linked or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Expression” refers to detectable production of a gene product by a cell. The gene product may be a transcription product (i.e., RNA), which may be referred to as “gene expression”, or the gene product may be a translation product of the transcription product (i.e., a protein), depending on the context.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

The term “derived from” is used herein to identify the original source of a molecule but is not meant to limit the method by which the molecule is made which can be, for example, by chemical synthesis or recombinant means.

A polynucleotide or nucleic acid “derived from” a designated sequence refers to a polynucleotide or nucleic acid that includes a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

A “barcode” refers to one or more nucleotide sequences that are used to identify a nucleic acid or cell with which the barcode is associated. Barcodes can be 3-1000 or more nucleotides in length, preferably 10-250 nucleotides in length, and more preferably 10-50 nucleotides in length, including any length within these ranges, such as 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, or 1000 nucleotides in length. Barcodes may be used, for example, to identify a single cell, subpopulation of cells, colony, or sample from which a nucleic acid originated. Barcodes may also be used to identify the identity, presence or position (i.e., positional barcode) of a nucleic acid, cell, colony, or sample from which a nucleic acid originated, such as the position of an insertion into a genome, a colony in a cellular array, the presence of donor DNA in a cell. For example, a barcode may be used to identify a genetically modified cell having a donor DNA encoded by a modified ncRNA. In some embodiments, a barcode is used to identify a particular type of genome edit or a particular type of donor nucleic acid.

The terms “hybridize” and “hybridization” refer to the formation of complexes between nucleotide sequences which are sufficiently complementary to form complexes via Watson-Crick base pairing.

The term “homologous region” refers to a region of a nucleic acid with homology to another nucleic acid region. Thus, whether a “homologous region” is present in a nucleic acid molecule is determined with reference to another nucleic acid region in the same or a different molecule. Further, since a nucleic acid is often double-stranded, the term “homologous, region,” as used herein, refers to the ability of nucleic acid molecules to hybridize to each other. For example, a single-stranded nucleic acid molecule can have two homologous regions which are capable of hybridizing to each other. Thus, the term “homologous region” includes nucleic acid segments with complementary sequences. Homologous regions may vary in length but will typically be between 4 and 500 nucleotides (e.g., from about 4 to about 40, from about 40 to about 80, from about 80 to about 120, from about 120 to about 160, from about 160 to about 200, from about 200 to about 240, from about 240 to about 280, from about 280 to about 320, from about 320 to about 360, from about 360 to about 400, from about 400 to about 440, etc.).

As used herein, the terms “complementary” or “complementarity” refers to polynucleotides that are able to form base pairs with one another. Base pairs are typically formed by hydrogen bonds between nucleotide units in an anti-parallel orientation between polynucleotide strands. Complementary polynucleotide strands can base pair in a Watson-Crick manner (e.g., A to T, A to U, C to G), or in any other manner that allows for the formation of duplexes. As persons skilled in the art are aware, when using RNA as opposed to DNA, uracil (U) rather than thymine (T) is the base that is considered to be complementary to adenosine. However, when uracil is denoted in the context of the present invention, the ability to substitute a thymine is implied, unless otherwise stated. “Complementarity” may exist between two RNA strands, two DNA strands, or between an RNA strand and a DNA strand. It is generally understood that two or more polynucleotides may be “complementary” and able to form a duplex despite having less than perfect or less than 100% complementarity. Two sequences are “perfectly complementary” or “100% complementary” if at least a contiguous portion of each polynucleotide sequence, comprising a region of complementarity, perfectly base pairs with the other polynucleotide without any mismatches or interruptions within such region. Two or more sequences are considered “perfectly complementary” or “100% complementary” even if either or both polynucleotides contain additional non-complementary sequences as long as the contiguous region of complementarity within each polynucleotide is able to perfectly hybridize with the other. “Less than perfect” complementarity refers to situations where less than all of the contiguous nucleotides within such region of complementarity are able to base pair with each other. Determining the percentage of complementarity between two polynucleotide sequences is a matter of ordinary skill in the art.

The term “donor polynucleotide” or “donor DNA” refers to a nucleic acid or polynucleotide that provides a nucleotide sequence of an intended edit to be integrated into the phage genome at a target locus.

A “target site” or “target sequence” is the nucleic acid sequence recognized (i.e., sufficiently complementary for hybridization) by a donor polynucleotide (donor DNA). For example, a target site can be a genomic site that is intended to be modified such as by insertion of one or more nucleotides, replacement of one or more nucleotides, deletion of one or more nucleotides, or a combination thereof.

The subject matter disclosed herein is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosed subject matter. The upper and lower limits of these smaller ranges may independently be included in the smaller ranges, and are also encompassed within the disclosed subject matter, subject to any specifically excluded limit in the stated range. Where the stated range includes one or both of the limits, ranges excluding either or both of those included limits are also included in the disclosed subject matter.

Unless defined otherwise, all technical and scientific terms used herein have the same meaning as commonly understood by one of ordinary skill in the art to which the disclosed subject matter belongs. Although any methods and materials similar or equivalent to those described herein can also be used in the practice or testing of the disclosed subject matter, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the nucleic acid” includes reference to one or more nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of any features or elements described herein, which includes use of a “negative” limitation.

It is appreciated that certain features of the disclosed subject matter, which are, for clarity, described in the context of separate embodiments, may also be provided in combination in a single embodiment. Conversely, various features of the disclosed subject matter, which are, for brevity, described in the context of a single embodiment, may also be provided separately or in any suitable sub-combination. All combinations of the embodiments pertaining to the disclosure are specifically embraced by the disclosed subject matter and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the disclosed subject matter is not entitled to antedate such publication. Further, the dates of publication provided may be different from the actual publication dates which may need to be independently confirmed.

The following Examples illustrate some of the materials, methods, and experiments that were used or performed in the development of the invention.

### Examples

### Introduction

Bacteriophages naturally control the composition of microbial ecosystems through selective infection of distinct bacterial species. Humans have long sought to harness this power of phages to make targeted interventions to the microbial world, such as delivering phages to a patient suffering from an infection to eliminate a bacterial pathogen. This approach to mitigate pathogenic bacterial infections, known as phage therapy, predates the discovery of penicillin, with 100 years of evidence for efficacy and safety⁴. However, the success of small molecule antibiotics over the same period of time has overshadowed and blunted innovation in phage therapy.

Unfortunately, it is now clear that reliance on small molecule antibiotics is not a permanent solution. Antimicrobial resistance in bacteria is associated with between 1-5 million deaths worldwide in 2019⁵, a figure that is projected to rise in the coming decades⁶. As such, there is a pressing need for alternatives or adjuvants to small-molecule antibiotics, like phage therapy, to avoid returning to the rampant morbidity of bacterial infections of the pre-antibiotic era. This work has already begun, with researchers and clinicians utilizing phage screening pipelines to identify natural phages for use in patients to overcome antimicrobial-resistant infections^{7,8}.

While these efforts demonstrate the potential of phage therapeutics, they do not scale well. Screening natural phages for each patient is time- and effort-intensive, requires massive repositories of natural phages, and results in the clinical use of biological materials that are not fully characterized¹. To functionally replace or supplement small-molecule antibiotics, phage therapy needs to be capable of industrialization and more rapid iteration. This will likely include modifying known phages to create engineered therapeutic tools that target specific pathogens and evade natural bacterial immunity, rather than just the opportunistic isolation of natural phages. However, such approaches are limited by the relative difficulty in modifying phage genomes¹.

The various approaches to modify phage genomes and the limitations imposed by each have been recently reviewed². One approach is to modify phage genomes by recombination within their bacterial host, which is inefficient and requires laborious screening of phage plaques. That screening effort can be reduced by imposing a counterselection on the unedited phage, such as CRISPR-based depletion of the wild-type phage⁹⁻¹². However, this negative selection is not universally applicable to all edit types because it requires functional disruption of a protospacer sequence¹³. Phages also frequently escape CRISPR targeting¹⁴, which can result in most selected phages containing escape mutations outside of the intended edit⁹. Another approach is to “reboot” a phage by assembling a modified phage genome in vitro and repackaging it in a host¹⁵, but such rebooting requires extensive work to enable in vitro assembly of a phage genome, and phage genome size limits the efficiency of transformation. A fully cell-free packaging system eliminates the issue of inefficient transformation¹⁶, but instead requires substantial upfront technical development, which is host species-specific.

Because of the urgent need for innovation in phage therapeutics and the clear technical hurdle in modifying phage genomes, we developed an alternative system for phage engineering. Our system edits phage genomes as they replicate within their bacterial hosts, by integrating a single-stranded DNA (ssDNA) donor encoding the edit into the replicating phage genome using a single-stranded annealing protein (SSAP) and single-stranded binding protein (SSB). A critical innovation is that we use a modified bacterial retron¹⁷⁻²⁰ to continuously produce the editing DNA donor by reverse transcription within the host. Thus, propagating a population of phage through this host strain enables continuous accumulation of the intended edit over generations within a single culture.

Moreover, this system enables a more complex form of editing in which the bacterial culture is composed of multiple, distinct editing hosts, each producing donors that edit different parts of the phage genome. Propagation of phages through such a complex culture leads to the accumulation of multiple distinct edits at distal locations in individual phage genomes. This approach is demonstrated herein, showing that (1) the editing is a continuous process in which edits accumulate over time; (2) it can be applied to multiple types of phage and used to introduce different edit types; (3) it can be optimized to reach efficiencies that do not require counter selection; and (4) it can be used to make multiplexed edits across a phage genome. For disambiguation with other techniques, this approach can be called phage retron recombineering, and term the molecular components that include a modified retron a recombitron.

### Example 1: Materials and Methods: Materials and Methods

This Example illustrates some of the materials and methods used in the development of the invention.

### Constructs and Strains

Experiments were carried out using a derivative of BL21-AI cells in which an endogenous retron was removed. The cells were modified to express Eco1 reverse transcriptases (RTs) along with the engineered ncRNAs. Expression was driven by T7/lac promoters from modified pET-21 (+) vectors (Twist). The ncRNA coding regions were modified using standard molecular cloning techniques. pORTMAGE-Ec1 plasmid was used to express CspRecT and mutLE32K (Addgene 138474). Phage stocks were purchased from ATCC and propagated through BL21 cells prior to experiments.

E. coli strains: NEB 5-alpha (NEB, C2987; not authenticated), BL21-AI (Thermo Fisher, C607003; not authenticated), bMS.346 and bSLS.114. bMS.346 (used previously²⁰) was generated from E. coli MG1655 by inactivating the exol and rec/genes with early stop codons. bSLS.114 (used previously²⁸) was generated from BL21-AI by deleting the retron Eco1 locus by lambda Red recombinase-mediated insertion of an FRT-flanked chloramphenicol resistance cassette, which was subsequently excised using FLP recombinase⁴¹. bCF.S was generated from bSLS. 114, also using the lambda Red system. A 12.1 kb region was deleted that contains a partial lambda*B prophage that is native to BL21-AI cells within the attB site, where temperate lambda integrates into the bacterial genome⁴².

Phage retron recombineering cultures were grown in LB, shaking at 37° C. with appropriate inducers and antibiotics. Inducers and antibiotics were used at the following working concentrations: 2 mg/ml L-arabinose (GoldBio, A-300), 1 mM IPTG (GoldBio, 12481C), 1 mM m-toluic acid (Sigma-Aldrich, 202-723-9), 35 μg/ml kanamycin (GoldBio, K-120), 100 μg/ml carbenicillin (GoldBio, C-103) and 25 μg/ml chloramphenicol (GoldBio, C-105; used at 10 μg/ml for selection during bacterial recombineering for strain generation).

### Plasmid Construction

All cloning steps were performed in E. coli NEB 5-alpha. pORTMAGE-Ec1 was generated previously (Addgene plasmid no. 138474) 26 Derivatives of pORTMAGE-Ec1 (pCF.109, pCF.110, pCF.111) were cloned to contain an additional SSB protein, amplified with PCR from its host genome, via Gibson Assembly. Plasmids for RT-Donor production, containing the retron-Eco1 RT and ncRNA with extended a1/a2 regions, were cloned from pSLS.492. pSLS.492 was generated previously (Addgene plasmid no. 184957) 2°. Specific donor sequences for small edits were encoded in primers and substituted into the RT-DNA-encoding region of the ncRNA with a PCR and KLD reaction (NEB M0554). Donor sequences for larger insertions were cloned through Gibson assembly, using synthesized gene fragments (Twist Biosciences).

### Phage Strains and Propagation

Phages were propagated from ATCC stocks (Lambda #97538, T7 #BAA-1025-B2, T5 #11303-B5, T2 #11303-B2) into a 2 mL culture of E. coli (BL214Eco1) at 37° C. at OD600 0.25 in LB medium supplemented with 0.1 mM MnCl₂ and 5 mM MgCl₂ (MMB) until culture collapse, according to established techniques^{43,44}. The culture was then centrifuged for 10 min at 4000 rpm and the supernatant was filtered through a 0.2 μm filter to remove bacterial remnants. Lysate titer was determined using the full plate plaque assay method as described by Kropinski et al.⁴⁵. Recombitrons were used to edit this lambda strain to encode two early stop codons in the cI gene, responsible for lysogeny control, to ensure the phage was strictly lytic (lambda ΔcI). After recombineering, plaques were Sanger sequenced to check the edit sites. An edited plaque was isolated and Illumina Miseq of its lysate was used to ensure purity of the edited phage. This strictly lytic version was used for all experiments involving lambda phage, unless otherwise noted.

Genomic locations used to label edits are from wildtype reference sequences of phages available through NCBI GenBank: lambda (J02459.1), T5 (AY587007.1), T7 (V01146.1), and T2 (AP018813.1). We found that the strain of phage lambda we used naturally contains a large genomic deletion between 21738 and 27723. This region encodes genes that are not well-characterized but may be involved in lysogeny^{46,47}.

### Plaque Assays

Small drop and full plate plaque assays were performed as previously described by Mazzocco et al.⁴⁸, starting from bacteria grown overnight at 37° C. For small drop plaque assays, 200 ul of the bacterial culture was mixed with 2 mL melted MMB agar (LB+0.1 mM MnCl₂+5 mM MgCl₂+0.75% agar) and plated on MMB agar plates. 10-fold serial dilutions in MMB were performed for each of the phages and 2 ul drops were placed on the bacterial layer. The plates were dried for 20 min at room temperature and then incubated overnight at 37° C. Full plate plaque assays were set up by mixing 200 ul of the bacterial culture with 20 ul of phage lysate, using 10-fold serial dilutions of the lysate to achieve between 200-10 plaques. After incubating at room temperature without shaking for 5 min, the mixture was added to 2 mL melted MMB agar and poured onto MMB agar plates. The plates were dried for 20 min at room temperature and incubated overnight at 37° C. Plaque forming units were counted to calculate the titer.

### Recombineering and Sequencing

The retron cassette, with modified ncRNA to contain a donor, was co-expressed with CspRecT and mutL E32K from the plasmid PORTMAGE-Ec1. All experiments, except multiplexing and large insertions/deletions (>30 bp), were conducted in 500 uL cultures in a deep 96-well plate. Multiplexing experiments were conducted in 3 mL cultures in 15 mL tubes. For amplification-free sequencing by nanopore, larger culture volumes (25 mL cultures in 250 mL flasks) were used to enable collection of a higher quantity of phage DNA. Cultures were induced for 2 hrs at 37° C., with shaking. The OD600 of each culture was measured to approximate cell density and cultures were diluted to OD600 0.25. Phages were originally propagated through the corresponding host that would be used for editing (B- or K-strain E. coli). A volume of pre-titered phage was added to the culture to reach a multiplicity of infection (MOI) of 0.1. The infected culture was grown overnight for 16 hrs, before being centrifuged for 10 min at 4000 rpm to remove the cells. The supernatant was filtered through a 0.2 μm filter to isolate phage.

For amplicon-based sequencing, the lysate was mixed 1:1 with DNase/RNase-free water and the mixture was incubated at 95° C. for 5 min. This boiled culture (0.25 ul) was used as a template in a 25 ul PCR reaction with primers flanking the edit site on the phage genome. These amplicons were indexed and sequenced on an Illumina MiSeq instrument. For amplification-free sequencing, extracellular DNA was removed through DNase I treatment, with 20 U of DNase I (NEB, M0303S) for 1 mL of phage lysate, incubated at room temperature for 15 min and then inactivated at 75° C. for 5 min. Phage were then lysed and DNA extracted using the Norgen Phage DNA Isolation Kit (Norgen, 46800). The samples were prepped for sequencing in a standard Nanopore workflow. DNA ends were repaired using NEBNext Ultra II End Repair/dA-Tailing Module (NEB, E7526S). End-repaired DNA was then cleaned up using Ampure XP beads. Barcodes were ligated using the standard protocol for Nanopore Barcode Expansion Kit (Oxford Nanopore Technologies, EXP-NBD196). After barcoding, the standard Oxford Nanopore adaptor ligation, clean-up, and loading protocols were followed for Ligation Sequencing Kit 109 and Flow Cell 106 for the MinION instrument (Oxford Nanopore Technologies, SQK-LSK109, FLO-MIN106D). Basccalling was performed using Guppy Basecaller with high accuracy and barcode trimming settings.

Sanger sequencing of phage plaques was accomplished by picking plaques produced from the full plate assay described above. Plates were sent to Azenta/Genewiz for sequencing with one of the MiSeq-compatible primers used to assess the same site. Sequences were analyzed using Geneious through alignment to the region surrounding the edit site on the phage genome.

### Editing Rate Quantification

A custom Python workflow was used to quantify edits from amplicon sequencing data. Reads were required to contain outside flanking nucleotide sequences that occur on the phage genome, but beyond the RT-Donor region to avoid quantifying RT-DNA. Reads were then trimmed by left and right sequences immediately flanking the edit site. Reads containing these inside flanking sequences in the correct order with an appropriate distance between them (depending on edit type) were assigned to either wild-type, edit, or other. The edit percentage is the number of edited reads over the sum of all reads containing flanking sequences.

A distinct Python program for quantifying amplification-free nanopore sequencing data, due to the higher error rates in nanopore sequencing and the lack of a defined region of the genome contained in each read. Reads were aligned using BLAST+ to three reference genomes: wildtype lambda, edited lambda containing the matching edit to the read's experiment, and BL21-AI E. coli. If reads aligned to either lambda genomes, the read's alignment coordinates had to be at least 50 bases past the insertion/deletion coordinates, as well as be >500 bp and have >50% of the read mapped to the reference genome as quality scores. If a read aligned to the insertion/deletion point and passed all quality scores, the percent identity and alignment length over read length were compared to assign the read as either wild-type or edited. Coverage of the edit region was 50-1000× per experimental condition.

### Example 2: System for Bacteriophage Modification

This Example illustrates some of the components and methods that can be used for phage modification.

The components of the following system were used: (1) a modified retron non-coding RNA (ncRNA) that contains both homology to the phage genome and the intended change to the phage genome; (2) a retron reverse-transcriptase (RT) protein to reverse-transcribe the retron ncRNA into a single-stranded DNA template for recombination; (3) a recT/single-stranded annealing protein (SSAP) to promote recombination of the single-stranded DNA template into the phage genome; and (4) a bacterial strain to harbor components 1, 2, and 3 that can be infected by the phage and carry out the editing during phage replication. Additional components that can facilitate use of the system included (5) a dominant-negative mutL to suppress mismatch recognition when making single-base or small changes; and (6) a single-stranded binding protein (SSB) that is compatible with the SSAP to promote recombination.

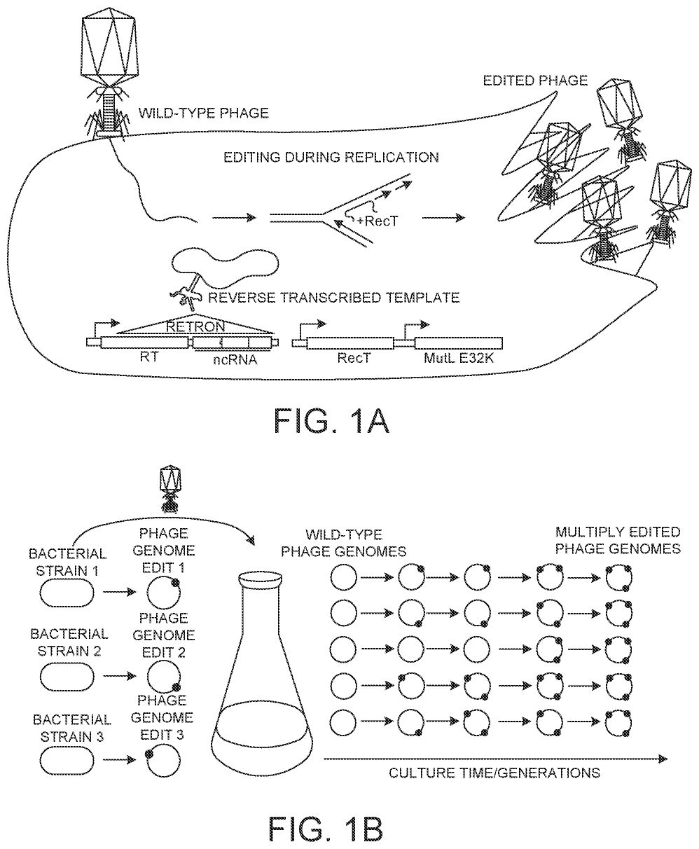

Host bacteria that had at least components 1, 2, and 3 are infected with phage, where components 1, 2, and 3 are listed in the paragraph above. Reverse-transcribed, retron-derived ssDNA can be integrated into the phage genome during replication of the phage by recT/SSAP, altering the phage genome in the process. When that replicated genome is repackaged, it results in a viable, edited phage (FIG. 1A). This process can be inefficient.

However, by propagating the phage through a population of bacteria that all express the editing components, edited phage genomes may be produced each time a phage passes through a cell. By performing such propagation, the proportion of edited phage genomes increases with the duration of culture. To introduce multiple edits, a single bacterial host can contain one or more modified retron ncRNAs that edit the phage genomes at different locations. In some cases, different cells within the bacterial population can harbor distinct, modified retron ncRNAs that edit at different locations along the phage genome. By culturing phage in bacterial populations of this type, the edits build up over time through multiple infection and replication cycles, enabling genome-wide engineering of phage genomes in a multiplexed system that requires little effort (FIG. 1B).

### Example 3: Retron-Based Modification of Lambda Phage

This example illustrated modification of lambda phage.

Retron ncRNA from retron-Eco1 was modified to edit the L protein stop codon in lambda phage. The editing system included use of E. coli BL21 bacterial host cells that endogenously expressed a SSB (single stranded binding) protein. The BL21 host cells were engineered to express an Eco1 reverse transcriptase (RT) and CspRecT (as a recT/SSAP, where SSAP is a DNA single-strand annealing protein that is compatible with CspRecT). The CspRecT increases the efficiency of single-locus editing in E. coli. The BL21 host cells additionally expressed a dominant-negative mutant form of mutL having the E32K mutation (which inhibits overall mismatch repair reaction as well as MutH activation). All of these gene components were expressed in the BL21 bacterial strain and the culture was infected with lambda phage.

After a day of culturing lambda phage with the modified E. coli BL21 bacterial host cells, phages were collected, and their genomes were sequenced using multiplexed Illumina sequencing.

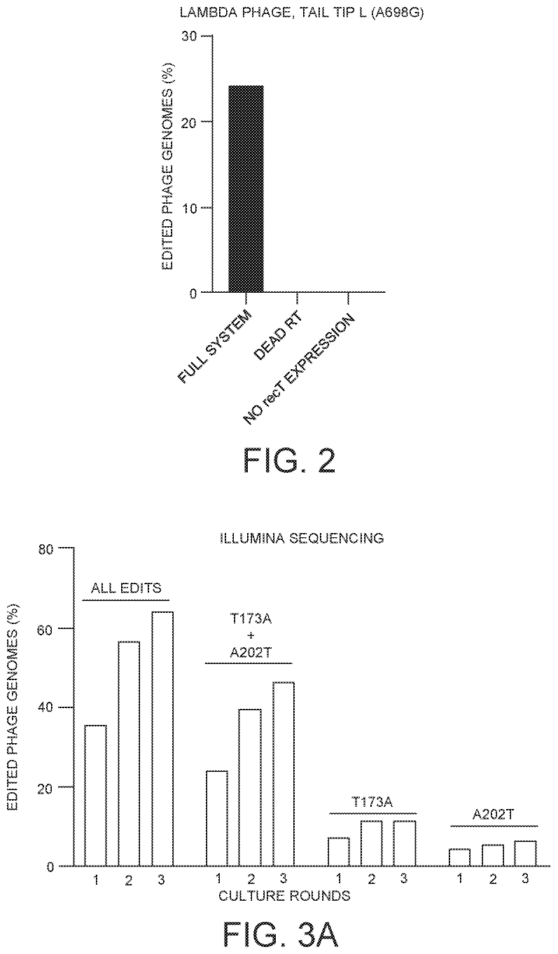

As illustrated in FIG. 2, approximately 24% of lambda genomes were precisely edited when the retron reverse transcriptase was expressed along with the other components described in this Example. Control cells that did not express RecT exhibited little or no phage editing (FIG. 2). In addition, editing was nearly eliminated in control cells expressing an inactive reverse transcriptase (FIG. 2). These data indicated that RecT is needed for efficient integration of the template via recombination. In addition, the results confirm that phage editing requires a reverse transcribed template, which had been provided in the modified retron-Eco1.

### Example 4: Improved Retron-Based Modification of Lambda Phage

This example illustrated improved efficiency of lambda phage modification, and modification of lambda DNA at two sites using a single type of retron ncRNA that provided the templets with two editing sites.

The procedures described in Examples 1 and 2 were employed to determine whether the percentage of phage genomes edited increases over time when phages are propagated through additional rounds of bacterial culture expressing the editing components. The bacterial host cells employed expressed an ncRNA designed to introduce two separate mutations to the cI gene in lambda phage.

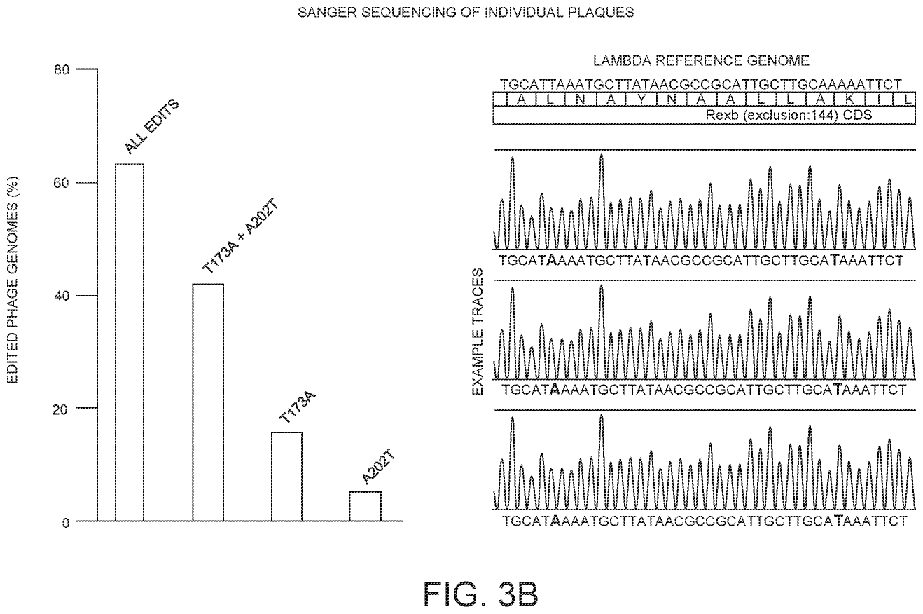

As shown in FIG. 3A, with each additional round of editing the total percentage of edited genomes increased. As illustrated in FIG. 3A, in most cases both edits were created in the individual genomes, while a smaller proportion of genomes acquired either one or the other edit. These edited genomes are packaged into viable, infectious phage as evidenced by the fact that the phage can be plated after editing onto a lawn of non-editing BL21 bacteria and isolate individual plaques could still be obtained that contain edited phage genomes in the same proportion as the edits observed in the bulk population (FIG. 3B).

### Example 5: Multiplexed Retron-Based Genome-Wide Editing of Phage

This Example further illustrates the potential of the methods and editing systems described herein for multiplexed genome-wide editing.

To evaluate multiplexed editing, lambda phage were cultured through a mixed population of bacterial host cells. The host cell population contained two separate bacterial strains, where strain 1 expressed an editing ncRNA with an A698G mutation and strain 2 expressed a different editing ncRNA with a C642T mutation.

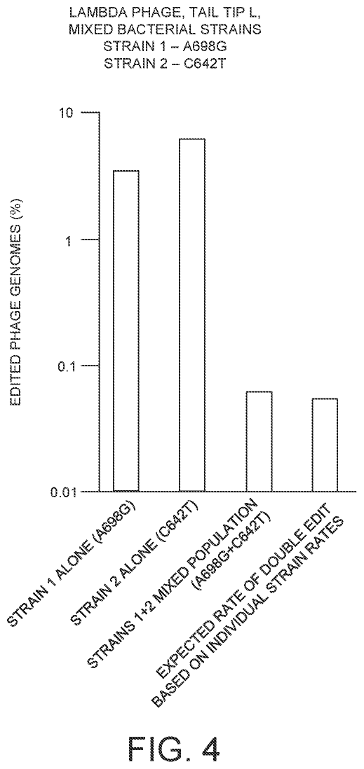

Each bacterial strain used in the population had first been separately tested to determine the editing efficiency of the individual ncRNAs. FIG. 4 illustrates the editing efficiencies of strains 1 and 2 alone.

The two strains were then mixed and lambda phage were propagated through this mixed population. After incubation, phage genomes were evaluated by sequencing. FIG. 4 illustrates the percent of edited phage genomes having both mutations.

Note that the percent of edited phage genomes having both mutations was almost exactly the optimal rate that had been calculated as possible, based on the editing efficiency of the individual strains.

### Example 6: Editing T5 Phage

This Example illustrates that the methods and editing components described herein can be used on phages other than lambda phage.

The editing systems and methods described herein (see Examples 1 and 2) were adapted and evaluated for editing T5 phage. The editing systems employed was the same as described in Examples 1 and 1, but modified ncRNAs were used that included donor DNAs for targeting the genome of phage T5.

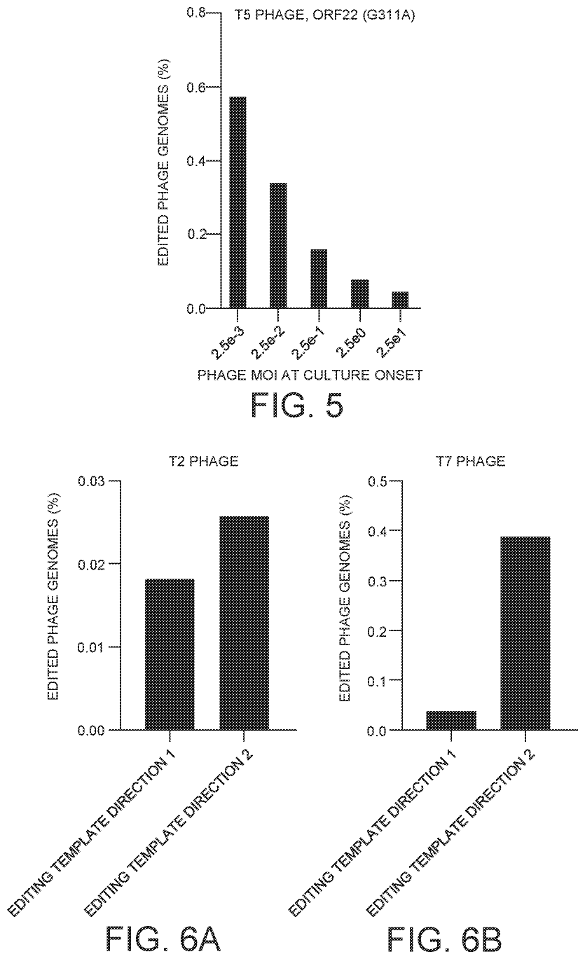

As illustrated in FIG. 5, the T5 genome was successfully edited. Also as illustrated in FIG. 5, the T5 editing rate was inversely correlated with the multiplicity of infection (MOI) at culture onset. The results further confirm that editing increased over time and with further phage generations.

In other experiments, the editing systems and methods described herein (see Examples 1 and 2) were adapted and evaluated for editing T2 and T7 phage. The editing systems employed was the same as described in Examples 1 and 1, but modified ncRNAs were used that included donor DNAs for targeting the genome of phage T2 or T7.

As illustrated in FIGS. 6A-6B, both of the T2 and T7 phage genomes were successfully edited. Moreover, FIG. 6A-6B illustrate that when recombineering with recT, more efficient editing occurs with an editing template in one direction than in the other. This occurs because the incorporation of the ssDNA occurs at the lagging strand during replication. Hence, the rate of editing depends on the direction of insertion of the editing template.

### Example 7: ncRNA/RT-DNA Features that can be Modified

The methods described herein can be used to evaluate features of the ncRNA that can be modified.

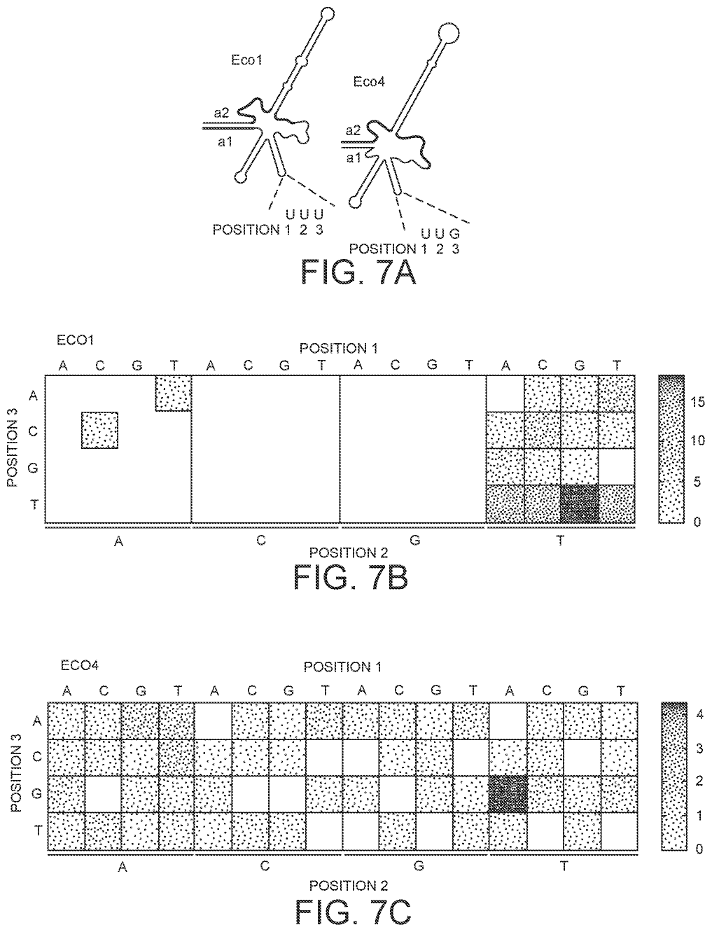

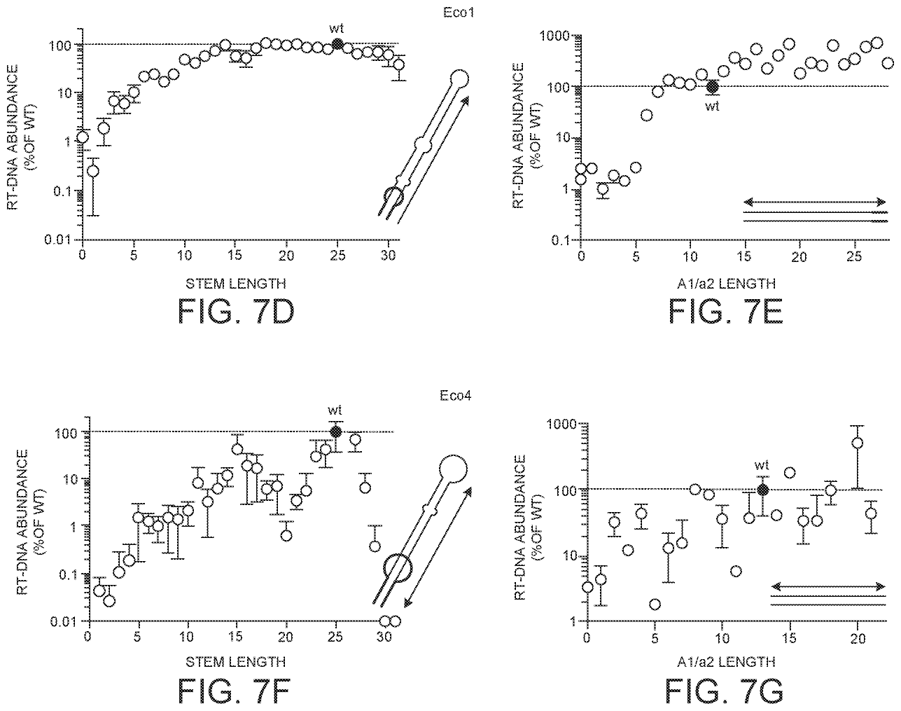

In one experiment, a loop region of the ncRNA was analyzed that was hypothesized to be involved in reverse transcriptase recognition. This loop region had a sequence that was somewhat different in Eco1 and Eco4 retrons (FIG. 7A). As illustrated in FIGS. 7A-7C, this loop was determined to be much more sensitive to modifications in Eco1 than Eco4.

In another experiment, the length of stem regions was evaluated to ascertain optimal stem lengths for retron ncRNAs. As shown in FIGS. 7D-7G, RT-DNA production from both Eco1 and Eco4 ncRNAs was negatively affected by reducing the stem length below about 15 base pairs and by reducing the length of the complementary region at the 5′ and 3′ ends of the ncRNA, termed a1/a2, below about 10 base pairs.

Interestingly, further experiments have shown that extension of the a1/a2 region can result in more than a ten-fold increase in RT-DNA production, which is the improvement that can be used to increase editing rates, for example, in a variety of cell types, including yeast.

### Example 8

### Recombitrons Target Phage Genomes for Editing

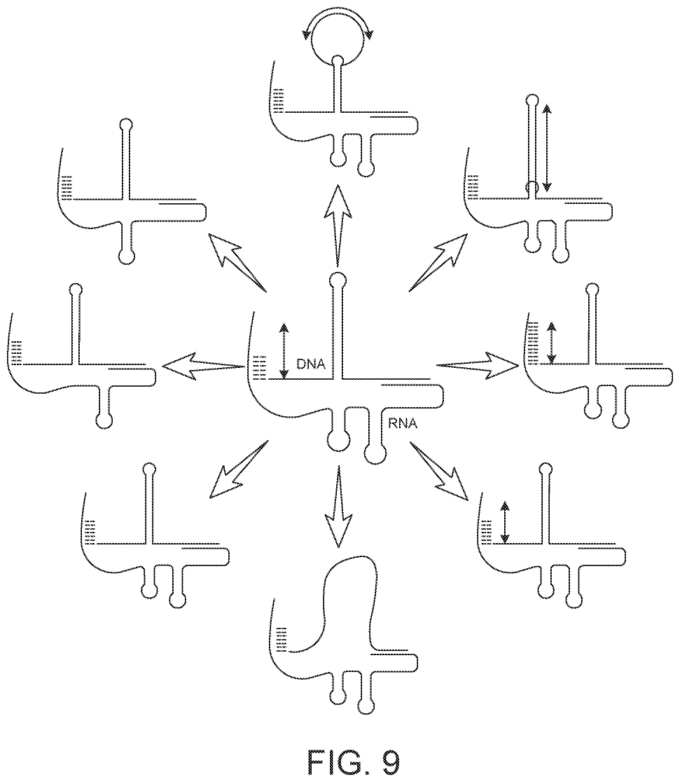

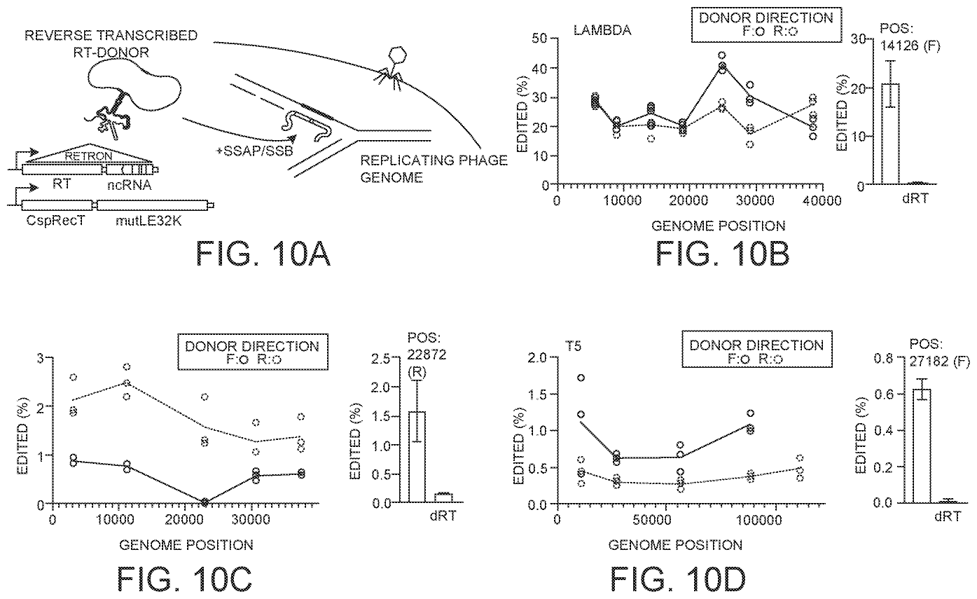

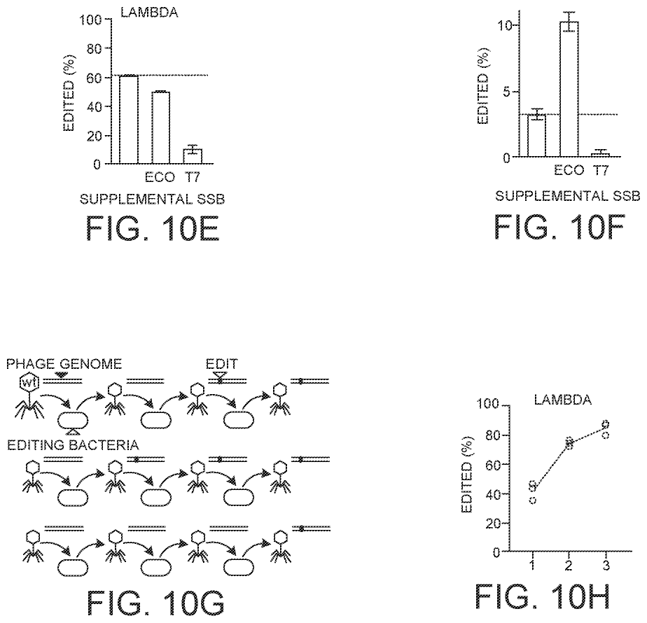

There are four core molecular components of a recombitron: a retron non-coding RNA (ncRNA) that is modified to encode an editing donor; a retron reverse transcriptase (RT); a single-stranded binding protein (SSB); and a single-stranded annealing protein (SSAP) (FIG. 10A). Endogenous retrons partially reverse transcribe a short (˜200 base) ncRNA into a single-stranded reverse-transcribed DNA (RT-DNA) of ˜90 bases. In bacteria, this RT-DNA is used in conjunction with retron accessory proteins to detect and respond to phage infection. The retron accessory proteins are necessary for the phage defense phenotype and are not included in the recombitron to avoid reconstituting an anti-phage system²¹⁻²³. Retron ncRNA was modified by adding nucleotides to the region that is reverse transcribed that are homologous to a locus in the phage genome and carry the edit to be incorporated.

The starting recombitron contains a modified retron-Eco1 ncRNA expressed on the same transcript as a retron-Eco1 RT, which will produce a 90-base-long reverse-transcribed editing donor (RT-Donor) inside the host bacteria. Once produced, the SSB will bind the editing RT-Donor to destabilize internal helices and promote interaction with an SSAP. In this case, the endogenous E. coli SSB was leveraged. Next, an SSAP promotes annealing of the RT-Donor to the lagging strand of a replication fork, where the sequence is incorporated into the newly replicated genome²⁴. From a separate promoter the SSAP CspRecT is expressed along with an optional recombitron element mutL E32K, a dominant-negative version of E. coli mutL that suppresses mismatch-repair²⁵⁻²⁷. Such suppression is needed when creating single-base mutations, but not required for larger insertions or deletions.

This system benefits from stacking several beneficial modifications to the retron and recombineering machinery discovered previously. The retron ncRNA, in addition to being modified to encode an RT-Donor, is also modified to extend the length of its a1/a2 region, which was previously found to increase the amount of RT-DNA produced^{20,28}. The SSAP, CspRecT, is more efficient than the previous standard, lambda β, and is known to be compatible with E. coli SSB²⁶. The dominant-negative mutL E32K eliminates the need to pre-engineer the host strain to remove mutS^{25,29}. A previous study attempted a single edit using a retron-produced donor against T5 phage, but only reported editing after counterselection¹². By using these stacked innovations, a system that does not require counter selection is produced.

To test whether recombitrons can be used to edit phages, recombitrons targeting 5-7 sites across the genome of four E. coli phages were designed: Lambda, T7, T5, and T2. Each recombitron was designed to make synonymous single-base substitutions to a stop codon, which could be fitness neutral. Separate recombitrons were constructed to produce the RT-Donor in either of the two possible orientations, given that the mechanism of retron recombineering requires integration into the lagging strand, and created a recombitron with a catalytically-dead RT at one position per phage as a control. The recombitron was pre-expressed for 2 hours in BL21-AI E. coli that lack the endogenous retron-Eco1 then phage was added at an MOI of 0.1 and the cultures were grown for 16 hours overnight. The next day, the cultures were spun down to collect phage in the supernatant. PCR was used to amplify the regions of interest in the phage genome (˜300 bases) surrounding the edit sites. These amplicons were sequenced on an Illumina MiSeq and quantified editing with custom software.

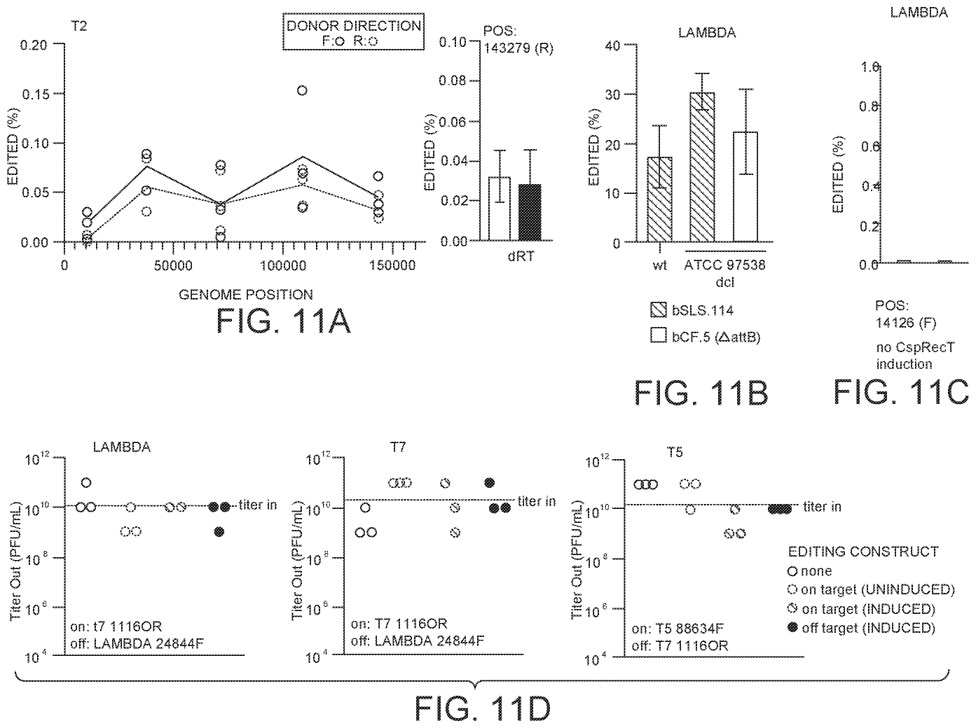

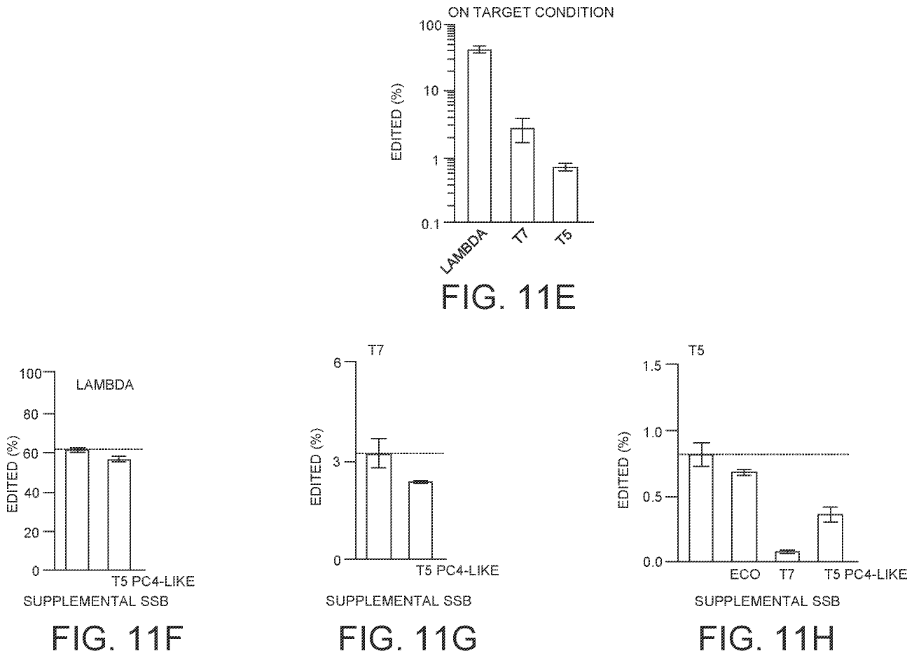

RT-dependent, counter-selection-free editing was observed in lambda (˜20%), T7 (˜1.5%), and T5 (˜0.6%) (FIG. 10B-D). We did not observe editing in T2 above the dead RT background level, which could be due to T2's use of modified 5-hydroxymethycytosines (FIG. 11A)³⁰. In lambda, T7, and T5, similar maximum editing efficiencies were obtained at each site across the genome. Editing efficiency was affected by RT-Donor direction in a manner generally consistent with individual phage replication mechanisms. Lambda has an early bidirectional phase of replication and a later unidirectional phase³¹. Thus, either strand may be lagging at some point and, accordingly, similar efficiencies were observed for the forward and reverse recombitron (FIG. 10B). T7 has a single origin of replication at one end of its linear genome, and directional editing was observed favoring the predicted lagging (reverse) strand (FIG. 10C) 32. Interestingly, T5 replication is less well studied, with one report describing bidirectional replication from multiple origins³³. However, a clear strand preference was found indicating dominant unidirectional replication from one end of the phage genome (FIG. 10D).

Editing efficiency differed among the phages tested, with lambda being the most efficiently edited. One possible explanation for this difference is that lambda is the only temperate phage tested, so hypothetically the editing could be occurring while lambda was integrated into the E. coli genome. However, two results were obtained inconsistent with this hypothesis. First, a lambda strain was generated with an inactivated cI gene that is needed for prophage maintenance and found similar levels of editing in that lambda strain as compared with the standard lambda strain (FIG. 11B)³⁴. Next, the bacterial host was modified to remove the lambda prophage integration site (attB), and again found similarly high levels of editing of the ΔcI strain in the attB⁻ bacteria (FIG. 11B)³⁵, Another possibility is that lambda β, an SSAP encoded by lambda, assists the recombitron²⁴. However, no editing of lambda was found in the absence of CspRecT expression, which is inconsistent with that possibility (FIG. 11C). Perhaps the recombitron differentially affects phage replication, which in turn affects editing efficiency. However, no effect was found on replication of the phage (as measured by phage titer) when the recombitron was included or expressed in the phage's bacterial host (FIG. 11D, E).

Another alternative explanation is SSB compatibility³⁶. Phage T7, unlike lambda, encodes a separate SSB in its genome (gp2.5)³⁷. Perhaps T7 SSB competes with the E. coli SSB for the recombitron RT-DNA, which could inhibit interactions with the CspRecT. To test this possibility, the lambda and T7 editing experiments were repeated at one locus each, while overexpressing either the E. coli or the T7 SSB. Consistent with this explanation, overexpression of the T7 SSB was found to have a large negative impact on editing of lambda, while the E. coli SSB had a much smaller impact (FIG. 10E). It was similarly found that overexpression of the T7 SSB strongly reduced editing of T7, whereas overexpression of the E. coli SSB had a large positive effect on T7 editing, more than doubling the efficiency to a rate of 10% (FIG. 10F). Thus, the T7 SSB appears to inhibit the retron recombineering approach but can be counteracted by overexpressing a compatible SSB. A similar approach was tried with T5, which has a much less characterized SSB (PC4-like, by homology to phage T438). While the T7 SSB similarly inhibited T5 editing, the T5 PC4-like protein had a small negative effect on editing, and the E. coli SSB did not improve T5 editing (FIG. 11F-H).

### Continuous Editing

One clear advantage of using recombitrons for phage editing is the fact that they edit continuously while the phage is propagating through the culture, increasing the proportion of edited phages with every generation (FIG. 10G). This is quite distinct from other methods of recombineering in phages where materials are delivered via transformation of the host at a single point in time³⁹. To illustrate the continuous nature of this approach, lambda was edited over three rounds of culture. Editing and analysis was performed as described above, but at the end of each round of editing, the resulting phage population was propagated through a fresh culture of editing the host. The percentage of edited genomes increased with each additional round, reaching >80% of phages edited after three rounds (FIG. 10H).

### Optimizing Recombitron Parameters

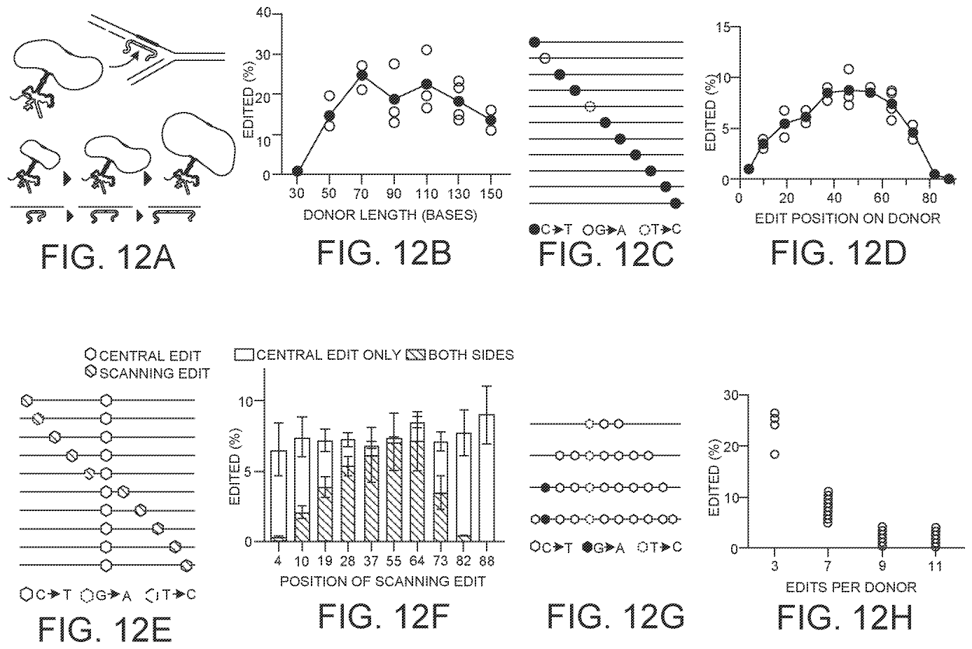

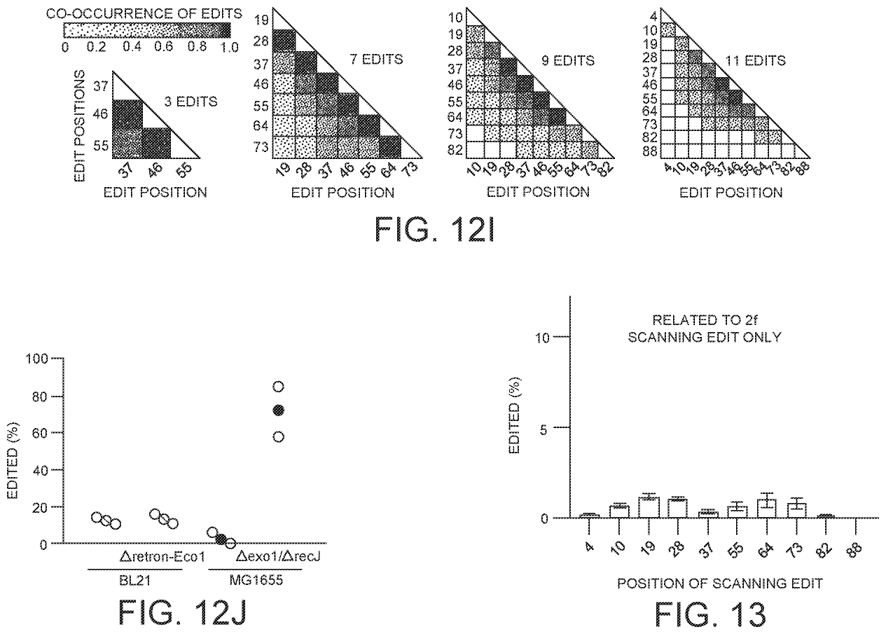

Given the initial success of recombitrons, the parameters of the system were next tested to achieve optimal editing. The first parameter tested was length of the RT-Donor. A set of recombitrons were designed with different RT-Donor lengths, each encoding a lambda edit (C14070T) in the center of the homologous donor (FIG. 12A). No editing with a 30 base RT-Donor was found, but all other lengths tested from 50 to 150 bases produced successful editing (FIG. 12B). The highest overall editing rates occurred with a 70 base RT-Donor. However, for donors between 50 and 150 bases, an analysis corrected for multiple comparisons only found a significant difference between 70 and 150 base donors, indicating that long RT-Donor length is detrimental to editing efficiency (FIG. 12B).

The next parameter tested was positioning of the edit within the RT-Donor. We tested a set of recombitrons where we held a 90-base region of homology to lambda constant and encoded an edit at different positions along the donor, each of which introduce a distinct synonymous single-base substitution (FIG. 12C). Here it was found that the editing rate increased as the edit approached the center of the donor (FIG. 12D). After correcting for multiple comparisons, no difference was found between edit placement between a central 27 bases from position 37 to 64, but a significant decline in editing outside the central region.

The effect of position when encoding multiple edits on a single RT-Donor was also tested. In this case, a set of recombitrons was tested with edits at different positions along the RT-Donor as above, but additionally included a central edit on every RT-Donor (FIG. 12E). Similar to the effect above, it was found that the rate of incorporating both of the edits on a single phage genome declined as the scanning edit approached either edge of the RT-Donor (FIG. 12F). However, there was a consistent editing rate for all recombitrons in phage genomes that were only edited at the central site. When both edits were positioned toward the center of the donor, most genomes contained both edits, whereas when the scanning edit was positioned toward the edge of the RT-Donor, there was a significant increase in likelihood of incorporating only the central edit (FIG. 12F). This result is interpreted as evidence that the RT-Donor can be partially used, with a bias toward using the central part of the RT-Donor. Only a very low rate of the scanning edit was found being incorporated without the central edit (FIG. 13).

Next, the effect of increasing the number of edits per recombitron was tested. A set of recombitrons was constructed with 3, 7, 9, or 11 of the scanning edits used above (FIG. 12G). It was found that editing with the 3-edit recombitron was comparable to the single edits previously tested, but editing rates declined across all edit sites when additional edits were encoded on a single recombitron (FIG. 12H). This is interpreted as an effect of decreasing homology across the donor, making it more difficult to anneal to the target site. To further assess which edits on the same RT-Donor were likely to be acquired together, the co-occurrence of edits was analyzed. Because of the effect of decreasing homology on the editing rate, the correlation coefficient r\hat{ }2 was calculated and normalized to the overall editing rate for each donor at each site. It was found that there is a bias toward acquiring the more central edits overall, and a tendency to co-edit nearby sites (FIG. 12I), again supporting the potential for partial use of the RT-Donor.

### Optimizing Host Strain

The effect of the editing host was also tested. Specifically, editing was compared in: B-strain E. coli (BL21-AI); a derivative of BL21-AI lacking the endogenous retron-Eco1; K-strain E. coli (MG1655); and derivative of MG1655 lacking Exol and RecJ-nucleases whose removal was previously shown to increase recombination rates using synthetic oligonucleotides 18,40. No difference was found between the BL21-AI and retron-deletion derivative, indicating that the endogenous retron does not interfere with the recombitron system (FIG. 12J). Decreased editing was found in the wild-type K-strain that was not statistically significant as compared to the B-strain, but significantly improved editing in the modified K-strain versus the B-strain (FIG. 12.J).

### Insertions and Deletions Via Recombitrons

Genomic deletions and insertions are also useful for engineered phage applications. Deletions can be used to remove potential virulence factors or eukaryotic toxins from phage genomes or to optimize phages by minimization. Insertions can be used to deliver cargo, such as nucleases that can help kill target cells, or anti-CRISPR proteins to escape phage defense systems. Therefore, the efficiency of engineering deletions and insertions of increasing size into the lambda genome was tested.

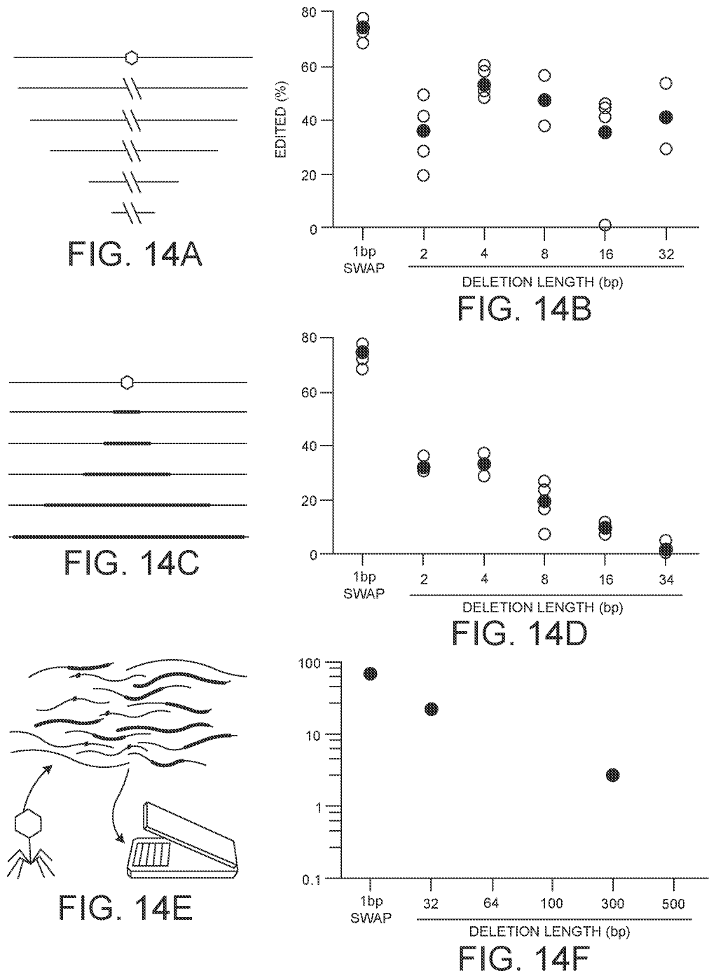

The efficiency of deleting 2, 4, 8, 16, or 32 bases was compared to one of the single base synonymous substitutions tested previously (FIG. 14A). These experiments were performed in the engineered K-strain that was found to yield higher editing efficiencies using inactivated cI lambda as the phage. Bases preceding position 37,673 were deleted using recombitrons where a 90 base RT-Donor contained flanking homology around the deletion site, but omitted the bases to be deleted. Consistent editing was found of ˜45% for each of the deletions (FIG. 14B) using Illumina amplicon sequencing. The deletion size did not significantly affect the efficiency of editing, although all the deletions exhibited significantly lower editing than the synonymous single base substitution.

Insertion of 2, 4, 8, 16, or 32 bases into the same lambda site was tested (FIG. 14C). Here, a range of editing efficiencies was found, from ˜32% for insertions of 2 bases to ˜10% for insertions of 16 bases (FIG. 14D). Like the deletions, all insertions were significantly less efficient than a synonymous single base substitution. Unlike the deletions, insertion size significantly affected efficiency, favoring smaller insertions.

Whereas the synonymous single base substitution is assumed to be neutral for phage fitness, the same cannot be assumed for the deletions and insertions, which could affect transcription or phage packaging. This may underlie the lower, although still substantial, rate of deletions and insertions overall. Additionally, the PCR required for analysis of editing by amplicon sequencing is known to be biased toward smaller amplicons, which could inflate the measured rates of the larger deletions and decrease the measured rates of larger insertions.

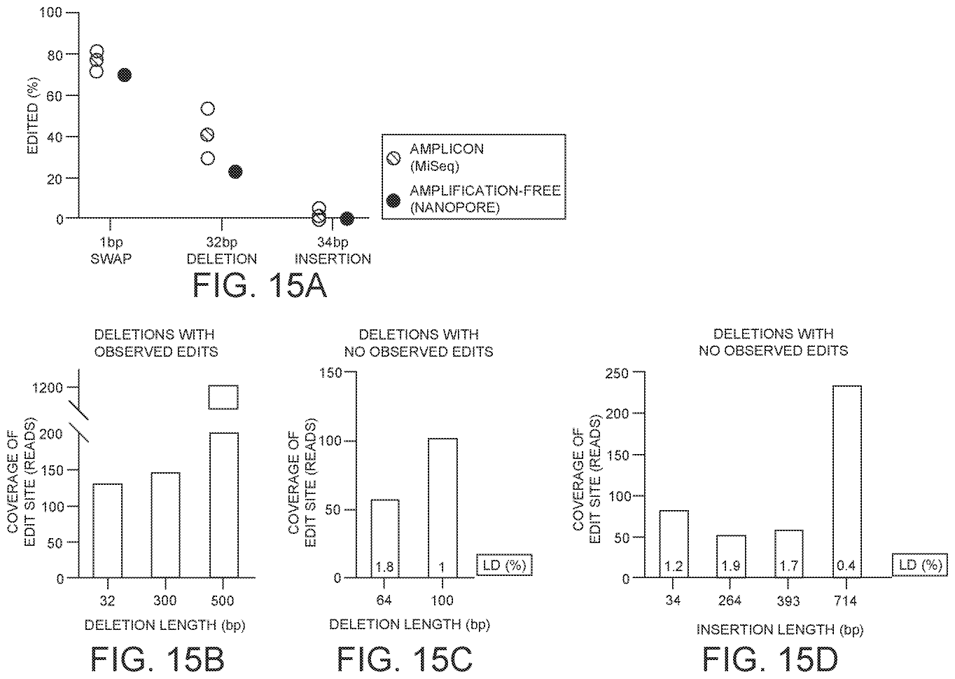

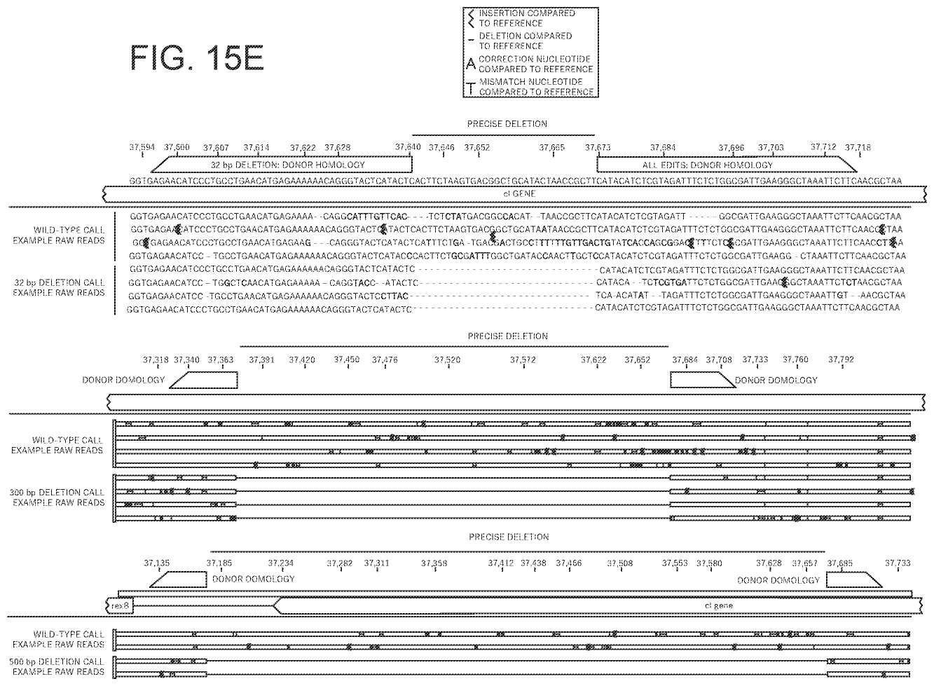

To test yet larger insertions in a manner that is not subject to the size bias of PCR, phages were edited and then their genomes were sequenced without amplification using long-read nanopore sequencing (FIG. 14E). After editing, phage genomes were isolated via Norgen Phage DNA Isolation kit, attached barcodes and nanopore adapters, and sequenced molecules for 24-48 h on a MinION (Oxford Nanopore Technologies). The resulting data was quantified using custom analysis software that binned reads by alignments to three possible genomes: a wild-type lambda genome, a lambda genome containing the relevant edit, or the BL21 E. coli genome as a negative control. BLAST alignment scores (percent identity, e-value, and alignment length) were compared for any read aligning to the lambda genome (wild-type or edited) in the region of the intended edit to quantify wild-type versus edited genomes. Consistent with a PCR bias for size, it was found that amplification-free sequencing resulted in comparable editing rates to amplification-based sequencing for quantifying a single base substitution, but slightly lower rates of editing when measuring a 32 base pair deletion as compared to amplification-based sequencing (FIG. 15A).

This amplification-free sequencing approach enabled for the extension of deletion and insertion size ranges. For deletions, 32, 64, 100, 300, or 500 base pairs were tested. Deletions of 32 bases were found at a frequency of ˜23%, 300 bases at a frequency of ˜2.7%, and 500 bases at a frequency of 0.16% (FIG. 14F). Deletions of 64 or 100 bases were not observed in this data. However, the long-read coverage of the editing region in these samples was limited, so one can only conclude that, if these edits occur, they are present at <1.8% and <1% respectively across the three replicates of the sequencing data (FIG. 15B,C). In cases where deletions were observed, they were reliably found at the intended location and of the intended size (FIG. 15E).

Insertion of larger sequences was also tested. Recombitrons were built to insert a 34 base frt recombination site, a 264 base anti-CRISPR protein (AcrIIA4), a 393 base anti-CRISPR protein (AcrIIA13), and a 714 base sfGFP. As anticipated from the low insertion rates of insertions >8 base pairs, none of these larger insertions were observed in the long-read sequencing data, which was read-limited to a detection level of ˜1% (FIG. 15D). Thus, the recombitrons enable high efficiency deletions of up to 32 base pairs and insertions of up to 8 base pairs but are not practical for counterselection-free isolation of larger deletions or insertions.

### Multiplexed Phage Engineering Via Recombitrons

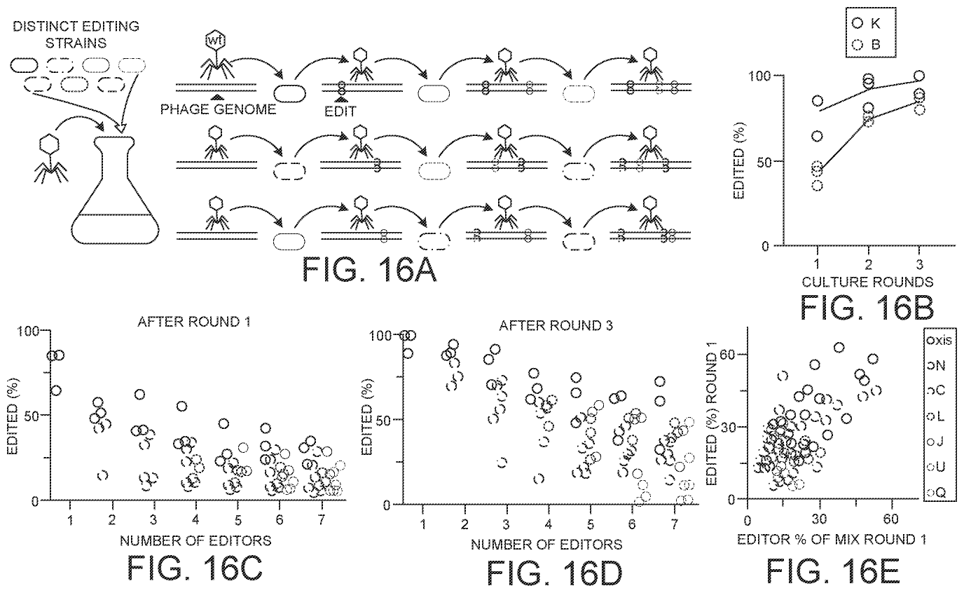

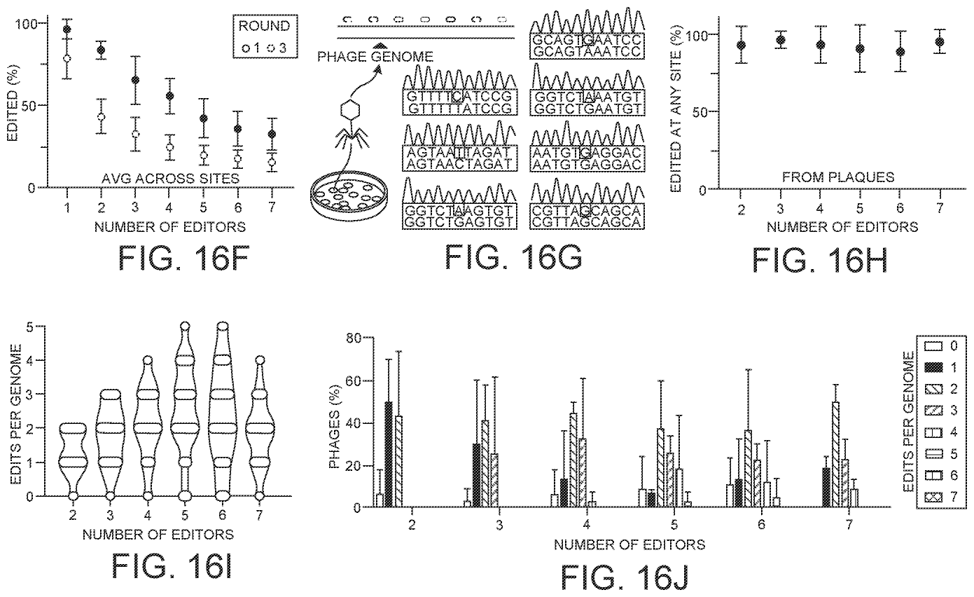

One shortcoming of all current phage editing approaches is an inability to simultaneously modify multiple sites in parallel on the same phage. Such parallel modification would be of great benefit to efforts aimed at engineering phages for targeted killing of pathogenic bacteria, but current approaches require cycles of editing, isolation, and re-editing that are impractical in an academic or industrial setting. It is reasoned that a slight modification of this recombitron approach would enable such parallel, multiplexed editing of distant sites across a genome. In this modification, multiple bacterial strains that each harbor distinct recombitrons targeting different parts of the phage genome are mixed in a single culture. The phage to be edited is then propagated through that mixed culture. Every new infection event is an opportunity to acquire an edit from one of the recombitrons and, over time, these distinct, distant edits accumulate on individual phage genomes (FIG. 16A). These experiments were ran in the engineered K-strain, through which the percentage of edited phage genomes increases with additional rounds of editing, beginning at a higher overall rate than the original experiments presented in FIG. 10. Editing lambda in this strain yields efficiencies that reach 100% after three rounds of culture (FIG. 16B).

Seven bacterial editing strains were created using the lambda recombitrons tested in FIG. 10B. Then made mixed cultures were made of 1, 2, 3, 4, 5, 6, or all 7 bacterial strains and performed editing of lambda phage in these mixtures. These strains were grown individually in liquid culture overnight, then separately pre-induced for 2 hours, before being diluted to OD 0.25 and then mixed together in equal proportions. These cultures were infected with lambda (ΔcI) at an MOI of 0.1, grew the cultures for 16 hours overnight, and collected the phage lysate the next morning. That phage stock was then used to perform two additional rounds of editing in mixed-host cultures prepared identically to those of the first round. Based on the results that the amount of phage put into the culture is roughly the amount of phage extracted (FIG. 11D,E), the same volume of phage was added in each round based on the titer determination at round one. Editing was quantified across phage genomes and genomic loci for each of the mixtures using Illumina sequencing. Editing across all sites from these mixed cultures was found after one round and increased editing in all cultures and sites after three rounds (FIG. 16C,D). It was found that the editing rate at any given site across all the mixtures was well correlated with the percentage of that editor in the mixture after round one (FIG. 16E), and that the overall editing rate across all sites declined with the number of recombitron strains used (FIG. 16F). This is consistent with a dilution effect of the strains on each other, which suggests that the overall editing rate is limited by the number of cells available for the phage to propagate through.

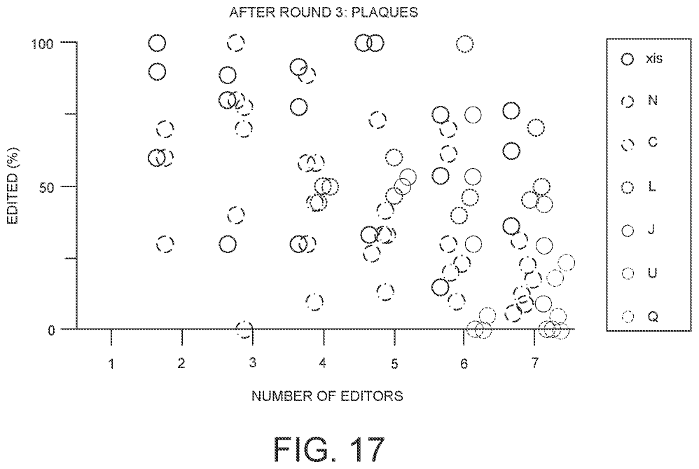

While amplicon sequencing showed substantial editing across many sites in a population of phages, it cannot be used to determine whether the edits are accumulating on individual phage genomes. Therefore, phages were plated after round three from each condition on bacterial lawns individual plaques were and Sanger sequenced (FIG. 16G). This showed that nearly 99.4% of phages were edited at one site or more, without employing any form of counter selection (FIG. 16H). The plaque-based analysis strongly mirrored the Illumina sequencing analysis of editing rate per site by mixture, when plaque data for a condition was pooled (FIG. 17). With the plaque data, however, one can also look at edits per phage genome. It was found that multiple sites were edited on a majority of the phages across all conditions, with individual phages edited at up to 5 distinct locations (FIG. 16I,J). This represents a new milestone in the continuous, multiplexed, selection-free engineering of phage genomes.

### DISCUSSION

Presented herein is a novel approach to phage editing using recombitrons that leverages modified retrons to continuously provide RT-Donors for recombineering in phage hosts. Recombitrons enable counter-selection-free generation of phage mutants across multiple phages, with optimized forms yielding up to 100% editing efficiency. Moreover, recombitrons can be multiplexed to generate multiple distant mutations on individual phage genomes. This approach is easy to perform. Recombitrons are generated from simple, standard cloning methods via inexpensive, short oligonucleotides. The process of editing merely requires propagating the bacteria/phage culture, with no intervening transformations or special reagents. Once recombitrons are cloned and phage stocks are prepared, the generation of lambda phage edited at up to five distinct positions required hands-on time of less than 2 hours.

This technical advance is poised to change the way we approach innovation in phage biology and phage therapeutics. For instance, studying epistasis in phage genomes becomes a feasible experiment that merely requires mixing recombitron strains in different combinations. The technical hurdle of phage library generation is also dramatically reduced. In the multiplexed experiment, 99.4% of phages were edited. Host range is a determinant of phage therapy efficacy, which could be addressed by rapidly screening a large library targeting the tail fiber or other host range genes for a fraction of the effort of current approaches.

### BIBLIOGRAPHY

1 Strathdee, S. A., et al. Phage therapy: From biological mechanisms to future directions. Cell 186, 17-31, doi: 10.1016/j.cell.2022.11.017 (2023).
2 Mahler, M., et al. Approaches for bacteriophage genome engineering. Trends in biotechnology, doi: 10.1016/j.tibtech.2022.08.008 (2022)
3 Simon, A. J., Ellington, A. D. & Finkelstein, I. J. Retrons and their applications in genome engineering. Nucleic Acids Res 47, 11007-11019, doi: 10.1093/nar/gkz865 (2019).
4 Żaczek, M., et al. Phage Therapy in Poland—a Centennial Journey to the First Ethically Approved Treatment Facility in Europe. Frontiers in microbiology 11, 1056, doi: 10.3389/fmicb.2020.01056 (2020).
5 Global burden of bacterial antimicrobial resistance in 2019: a systematic analysis. Lancet (London, England) 399, 629-655, doi: 10.1016/s0140-6736 (21) 02724-0 (2022).
6 O'Neill, J. Tackling drug-resistant infections globally: final report and recommendations. (2016).
7 Chan, B. K., et al. Bacteriophage therapy for infections in CF. Pediatric pulmonology 56 Suppl 1, S4-s9, doi: 10.1002/ppul.25190 (2021).
8 Schooley, R. T. et al. Development and Use of Personalized Bacteriophage-Based Therapeutic Cocktails To Treat a Patient with a Disseminated Resistant Acinetobacter baumannii Infection. Antimicrobial agents and chemotherapy 61, doi: 10.1128/aac.00954-17 (2017).
9 Kiro, R., et al. Efficient engineering of a bacteriophage genome using the type I-E CRISPR-Cas system. RNA biology 11, 42-44, doi: 10.4161/rna.27766 (2014).
10 Box, A. M., et al. Functional Analysis of Bacteriophage Immunity through a Type I-E CRISPR-Cas System in Vibrio cholerae and Its Application in Bacteriophage Genome Engineering. Journal of bacteriology 198, 578-590, doi: 10.1128/jb.00747-15 (2016).
11 Bari, S. M. N., et al. Strategies for Editing Virulent Staphylococcal Phages Using CRISPR-Cas10. ACS Synth Biol 6, 2316-2325, doi: 10.1021/acssynbio. 7600240 (2017).
12 Ramirez-Chamorro, L., Boulanger, P. & Rossier, O. Strategies for Bacteriophage T5 Mutagenesis: Expanding the Toolbox for Phage Genome Engineering. Frontiers in microbiology 12, 667332, doi: 10.3389/fmich.2021.667332 (2021).
13 Adler, B. A. et al. Broad-spectrum CRISPR-Cas13a enables efficient phage genome editing. Nature microbiology 7, 1967-1979, doi: 10.1038/s41564-022-01258-x (2022)
14 Strotskaya, A. et al. The action of Escherichia coli CRISPR-Cas system on lytic bacteriophages with different lifestyles and development strategies. Nucleic Acids Res 45, 1946-1957, doi: 10.1093/nar/gkx042 (2017).
15 Ando, H., et al. Engineering Modular Viral Scaffolds for Targeted Bacterial Population Editing. Cell systems 1, 187-196, doi: 10.1016/j.cels.2015.08.013 (2015).
16 Emslander, Q. et al. Cell-free production of personalized therapeutic phages targeting multidrug-resistant bacteria. Cell chemical biology 29, 1434-1445.e1437, doi: 10.1016/j.chembiol.2022.06.003 (2022).
17 Farzadfard, F. & Lu, T. K. Genomically encoded analog memory with precise in vivo DNA writing in living cell populations. Science 346, 1256272-1256272, doi: 10.1126/science. 1256272 (2014).
18 Schubert, M. G. et al. High-throughput functional variant screens via in vivo production of single-stranded DNA. PNAS 118, e2018181118, doi: 10.1073/pnas.2018181118 (2021).
19 Simon, A. J., Morrow, B. R. & Ellington, A. D. Retroelement-Based Genome Editing and Evolution. ACS Synth. Biol. 7, 2600-2611, doi: 10.1021/acssynbio.8b00273 (2018).
20 Lopez, S. C., et al. Precise genome editing across kingdoms of life using retron-derived DNA. Nat Chem Biol 18, 199-206, doi: 10.1038/s41589-021-00927-y (2022).
21 Bobonis, J. et al. Bacterial retrons encode phage-defending tripartite toxin-antitoxin systems. Nature 609, 144-150, doi: 10.1038/s41586-022-05091-4 (2022).
22 Millman, A. et al. Bacterial Retrons Function In Anti-Phage Defense. Cell 183, 1551-1561.e1512, doi: 10.1016/j.cell.2020.09.065 (2020).
23 Palka, C., et al. Retron reverse transcriptase termination and phage defense are dependent on host RNase H1. Nucleic Acids Res 50, 3490-3504, doi: 10.1093/nar/gkac177 (2022).
24 Mosberg, J. A., Lajoie, M. J. & Church, G. M. Lambda red recombineering in Escherichia coli occurs through a fully single-stranded intermediate. Genetics 186, 791-799, doi: 10.1534/genetics.110.120782 (2010).
25 Nyerges, A. et al. A highly precise and portable genome engineering method allows comparison of mutational effects across bacterial species. Proceedings of the National Academy of Sciences of the United States of America 113, 2502-2507, doi: 10.1073/pnas.1520040113 (2016).
26 Wannier, T. M. et al. Improved bacterial recombineering by parallelized protein discovery. Proceedings of the National Academy of Sciences of the United States of America 117, 13689-13698, doi: 10.1073/pnas.2001588117 (2020).
27 Nyerges, A. et al. Conditional DNA repair mutants enable highly precise genome engineering. Nucleic Acids Res 42, e62, doi: 10.1093/nar/gku105 (2014).
28 Bhattarai-Kline, S. et al. Recording gene expression order in DNA by CRISPR addition of retron barcodes. Nature 608, 217-225, doi: 10.1038/s41586-022-04994-6 (2022).
29 Aronshtam, A. & Marinus, M. G. Dominant negative mutator mutations in the mutL gene of Escherichia coli. Nucleic Acids Res 24, 2498-2504, doi: 10.1093/nar/24.13.2498 (1996).
30 Weigele, P. & Raleigh, E. A. Biosynthesis and Function of Modified Bases in Bacteria and Their Viruses. Chemical reviews 116, 12655-12687, doi: 10.1021/acs.chemrev.6b00114 (2016)
31 Weigel, C. & Seitz, H. Bacteriophage replication modules. FEMS microbiology reviews 30, 321-381, doi: 10.1111/j. 1574-6976.2006.00015.x (2006).
32 Wolfson, J., Dressler, D. & Magazin, M. Bacteriophage T7 DNA replication: a linear replicating intermediate (gradient centrifugation-electron microscopy-E. coli-DNA partial denaturation). Proceedings of the National Academy of Sciences of the United States of America 69, 499-504, doi: 10.1073/pnas.69.2.499 (1972).
33 Bourguignon, G. J., Sweeney, T. K. & Delius, H. Multiple origins and circular structures in replicating T5 bacteriophage DNA. Journal of virology 18, 245-259, doi: 10.1128/jvi.18.1.245-259.1976 (1976).
34 Hochschild, A. & Lewis, M. The bacteriophage lambda CI protein finds an asymmetric solution. Current opinion in structural biology 19, 79-86, doi: 10.1016/j.sbi.2008.12.008 (2009).
35 Tal, A., et al. Location of the unique integration site on an Escherichia coli chromosome by bacteriophage lambda DNA in vivo. Proceedings of the National Academy of Sciences of the United States of America 111, 7308-7312, doi: 10.1073/pnas. 1324066111 (2014).
36 Filsinger, G. T. et al. Characterizing the portability of phage-encoded homologous recombination proteins. Nat Chem Biol 17, 394-402, doi: 10.1038/s41589-020-00710-5 (2021).
37 Hernandez, A. J. & Richardson, C. C. Gp2.5, the multifunctional bacteriophage T7 single-stranded DNA binding protein. Seminars in cell & developmental biology 86, 92-101, doi: 10.1016/j.semcdb.2018.03.018 (2019).
38 Werten, S. Identification of the ssDNA-binding protein of bacteriophage T5: Implications for T5 replication. Bacteriophage 3, e27304, doi: 10.4161/bact.27304 (2013).
39 Marinelli, L. J. et al. BRED: a simple and powerful tool for constructing mutant and recombinant bacteriophage genomes. PLOS ONE 3, e3957, doi: 10.1371/journal.pone.0003957 (2008).
40 Mosberg, J. A., et al. Improving Lambda Red Genome Engineering in Escherichia coli via Rational Removal of Endogenous Nucleases. PLOS ONE 7, e44638, doi: 10.1371/journal.pone. 0044638 (2012).
41 Datsenko, K. A. & Wanner, B. L. One-step inactivation of chromosomal genes in Escherichia coli K-12 using PCR products. PNAS 97, 6640-6645, doi: 10.1073/pnas. 120163297 (2000).
42 Jeong, H., Kim, H. J. & Lee, S. J. Complete Genome Sequence of Escherichia coli Strain BL21. Genome announcements 3, doi: 10.1128/genomeA.00134-15 (2015).
43 Doron, S. et al. Systematic discovery of antiphage defense systems in the microbial pangenome. Science 359, doi: 10.1126/science aar4120 (2018).
44 Fortier, L. C. & Moineau, S. Phage production and maintenance of stocks, including expected stock lifetimes. Methods in molecular biology (Clifton, N.J.) 501, 203-219, doi: 10.1007/978-1-60327-164-6 19 (2009).
45 Kropinski, A. M., et al. Enumeration of bacteriophages by double agar overlay plaque assay. Methods in molecular biology (Clifton, N.J.) 501, 69-76, doi: 10.1007/978-1-60327-164-6_7 (2009).
46 Rajagopala, S. V., Casjens, S. & Uetz, P. The protein interaction map of bacteriophage lambda. BMC microbiology 11, 213, doi: 10.1186/1471-2180-11-213 (2011).
47 Epp, C., Pearson, M. L. & Enquist, L. Downstream regulation of int gene expression by the b2 region in phage lambda. Gene 13, 327-337, doi: 10.1016/0378-1119 (81) 90012-3 (1981).
48 Mazzocco, A., et al. Enumeration of bacteriophages using the small drop plaque assay system. Methods in molecular biology (Clifton, N.J.) 501, 81-85, doi: 10.1007/978-1-60327-164-6 9 (2009).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A method comprising incubating at least one population of bacteriophages (phages) with a population of modified bacterial host cells, wherein the modified bacterial host cells comprise one or more types of reverse transcriptases and at least one expression cassette comprising a promoter operably linked to a segment encoding a retron non-coding RNA (ncRNA) encoding one or more donor DNAs adapted for editing phage genomes, to thereby generate a population of phages comprising genomically edited phages.

2. The method of claim 1, wherein the population of modified bacterial host cells is a homogenous population of modified bacterial host cells, each comprising an expression cassette comprising a promoter operably linked to a segment encoding one type of retron non-coding RNA (ncRNA) encoding one type of donor DNAs adapted for editing phage genomes.

3. The method of claim 1, wherein the population of modified bacterial host cells is a heterogenous population of modified bacterial host cells, wherein one or more types of the host cells comprise different expression cassettes adapted for expressing different types of retron non-coding RNA (ncRNA) encoding different types of donor DNAs for editing phage genomes.

4. The method of claim 1, further comprising incubating the population of phages with a second population of modified bacterial host cells to generate a second population of phages comprising additional genomic edited phages.

5. The method of claim 4, further comprising incubating the population of phages or the second population of phages sequentially with a third or more populations of modified bacterial host cells to generate one or more subsequent populations of phages each comprising additional genomic edited phages.

6. The method of claim 5, wherein the population of modified bacterial host cells, the homogenous population of modified bacterial host cells, second population of modified bacterial host cells, the heterogenous population of modified bacterial host cells, the third or more populations of modified bacterial host cells were without bacteriophages prior to the incubating step(s).

7. The method of claim 1, wherein the bacterial host cells further comprise one or more expression cassettes, each expression cassette comprising a promoter operably linked to one or more segments encoding one or more types of reverse transcriptases, one or more types of single strand annealing proteins (SSAPs), one or more types of single-stranded DNA binding proteins (SSBs), one or more mutant mismatch repair proteins, or combinations thereof.

8. (canceled)

9. The method of claim 7, wherein the one or more single strand annealing proteins (SSAPs) comprise one or more types of RecT recombinases.

10. The method of claim 7, wherein the bacterial host cells further comprise one or more single-stranded DNA binding proteins (SSBs).

11. The method of claim 1, wherein each of the modified bacterial host cells comprise one or more types of reverse transcriptases.

12. The method of claim 11, wherein one or more types of the reverse transcriptases comprise retron reverse transcriptases.

13. The method of claim 7, wherein the one or more types of one or more single strand annealing proteins (SSAPs) comprise one or more bacterial species single strand annealing proteins (SSAPs).

14. The method of claim 7, wherein the one or more types of single strand annealing proteins (SSAPs) comprise bacteriophage SSAPs.

15. The method of claim 7, wherein the one or more types of single single-stranded DNA binding proteins (SSBs) comprise bacterial SSBs.

16. The method of claim 7, wherein at least one or more mutant mismatch repair proteins is a dominant-negative mutant mutL.

17. The method of claim 7, wherein at least one or more mutant mismatch repair proteins is a E32K mutant mutL protein, or a mutL homolog with a lysine substituted for a glutamic acid in an amino acid position homologous to an E. coli mutL E32K mutation.

18. The method of claim 5, wherein the population of phages, second population of phages, third population of phages, or subsequent populations of phages comprise one genomically edited site.

19. The method of claim 5, wherein the population of phages, second population of phages, third population of phages, or subsequent populations of phages comprise more than one genomically edited site.

20. A bacterial host cell comprising one or more reverse transcriptases and one or more recombinantly expressed types of retron non-coding RNAs (ncRNAs), each ncRNA encoding one or more donor DNAs adapted for editing phage genomes.

21. The bacterial host cell of claim 20, further comprising at least one endogenously or recombinantly expressed single strand annealing protein (SSAP), single-stranded DNA binding protein (SSB), mutant mismatch repair protein, or a combination thereof.

22. The bacterial host cell of claim 20, wherein at least one single strand annealing protein (SSAP) is a RecT recombinase.


SEQUENCE LISTING (49 sequences)

SEQ ID NO: 1 - protein, 261 aa, Bacteriophage lambda
MSTALATLAGKLAERVGMDSVDPQELITTLRQTAFKGDASDAQFIALLIVANQYGLNPWTKEIYAFPDKQNGIVPVVGVDGWSRIINENQQFDGMDFEQDNESCTCRIYRKDRNHPICVTEWMDECRREPFKTREGREITGPWQSHPKRMLRHKAMIQCARLAFGFAGIYDKDEAERIVENTAYTAERQPERDITPVNDETMQEINTLLIALDKTWDDDLLPLCSQIFRRDIRASSELTQAEAVKALGFLKQKAAEQKVAA

SEQ ID NO: 2 - protein, 261 aa, Shigella dysenteriae
MSTALATLAGKLAERVGMDSVDPQELITTLRQTAFKGDASDAQFIALLIVANQYGLNPWTKEIYAFPDKQNGIVPVVGVDGWSRIINENQQFDGMDFEQDNESCTCRIYRKDRNHPICVTEWMDECRRAPFKTREGREITGPWQSHPKRMLRHKAMIQCARLAFGFAGIYDKDEAERIVENTAYTTERQPERDITPVNEETMSEINALLTFMEKTWDDDLLPLCSQIFRRNIYTSSELTQAEAVKVLGFLKQKVTEQKVAA

SEQ ID NO: 3 - protein, 261 aa, Escherichia phage
MSTALATLAGKLAERVGMDSVDPQELITTLRQTAFKGDASDAQFIALLIVANQYGLNPWTKEIYAFPDKQNGIVPVVGVDGWSRIINENQQFDGMDFEQDNESCTCRIYRKDRNHPICVTEWMDECRREPFKTREGREITGPWQSHPKRMLRHKAMIQCARLAFGFAGIYDKDEAERIVENTAYTAERQPERDITPVNDETMQEINTLLIALDKTWDDDLLPLCSQIFRRDIRASSELTQAEAVKVLGFLKQKASEQKVAA

SEQ ID NO: 4 - protein, 261 aa, Enterobacteria phage; identical to SEQ ID NO: 3

SEQ ID NO: 5 - protein, 261 aa, Escherichia phage
MSTALATLAGKLAERVGMDSVDPQELITTLRQTAFKGDASDAQFIALLIVANQYGLNPWTKEIYAFPDKQNGIVPVVGVDGWSRIINENQQFDGMDFEQDNESCTCRIYRKDRNHPICVTEWMDECRREPFKTREGREITGPWQSHPKRMLRHKAMIQCARLAFGFAGIYDKDEAERIVENTAYTAERQPERDITPVNDETMQEINTLLIALDKTWDDDLLPLCSQIFRRDIRASSELTQAEAVKALGFLKQKATEQKVAA

SEQ ID NO: 6 - protein, 279 aa, Serratia phage
MSTALAAIAQSSGVSVDDVTDVLKGMIISAKNQHGAQVSNAELAVVSGVCAKYDLNPMVKECAAFISGGKLQVVLMIDGWYRIVNRQPNFDGVEFDDHIDDKSVLTAITCRMYIKGRTRPVVVTEYMSECRDPKSSVWQKWPARMLRHKAYIQCARMTFGISDMIDNDEASRITQGEKNITQQASSVSTVDYQAIDQAMGECEDHDALNKLCAEIRAEMEKRGTWNSEKVTLADMKSRHKARIDAAVVTDEFEVVEDDNDGAVKSDVEDSATDDDVPFE

SEQ ID NO: 7 - protein, 269 aa, Escherichia coli
MTKQPPIAKADLQKTQGNRAPAAVKNSDVISFINQPSMKEQLAAALPRHMTAERMIRIATTEIRKVPALGNCDTMSFVSAIVQCSQLGLEPGSALGHAYLLPFGNKNEKSGKKNVQLIIGYRGMIDLARRSGQIASLSARVVREGDEFSFEFGLDEKLIHRPGENEDAPVTHVYAVARLKDGGTQFEVMTRKQIELVRSLSKAGNNGPWVTHWEEMAKKTAIRRLFKYLPVSIEIQRAVSMDEKEPLTIDPADSSVLTGEYSVIDNSEE

SEQ ID NO: 8 - DNA, 810 bp, Escherichia coli
  source 1..810: mol_type = other DNA
atgactaagcaaccaccaatcgcaaaagccgatctgcaaaaaactcagggaaaccgtgcaccagcagcagttaaaaatagcgacgtgattagttttattaaccagccatcaatgaaagagcaactggcagcagctcttccacgccatatgacggctgaacgtatgatccgtatcgccaccacagaaattcgtaaagttccggcgttaggaaactgtgacactatgagttttgtcagtgcgatcgtacagtgttcacagctcggacttgagccaggtagcgccctcggtcatgcatatttactgccttttggtaataaaaacgaaaagagcggtaaaaagaacgttcagctaatcattggctatcgcggcatgattgatctggctcgccgttctggtcaaatcgccagcctgtcagcccgtgttgtccgtgaaggtgacgagtttagcttcgaatttggccttgatgaaaagttaatacaccgcccgggagaaaacgaagatgccccggttacccacgtctatgctgtcgcaagactgaaagacggaggtactcagtttgaagttatgacgcgcaaacagattgagctggtgcgcagcctgagtaaagctggtaataacgggccgtgggtaactcactgggaagaaatggcaaagaaaacggctattcgtcgcctgttcaaatatttgcccgtatcaattgagatccagcgtgcagtatcaatggatgaaaaggaaccactgacaatcgatcctgcagattcctctgtattaaccggggaatacagtgtaatcgataattcagaggaataa

SEQ ID NO: 9 - protein, 269 aa, Escherichia coli; identical to SEQ ID NO: 7

SEQ ID NO: 10 - protein, 269 aa, Escherichia ruysiae
MTKQPPIAKADLQKTQGNRAPAAVKNSDVISFINQPSMKEQLAAALPRHMTAERMIRIATTEIRKVPALGNCDTMSFVSAIVQCSQLGLEPGSALGHAYLLPFGNKNEKSGKKNVQLIIGYRGMIDLARRSGQIASLSARIVREGDEFSFEFGLDEKLIHRPGENEDAPVTHVYAVARLKDGGTQFEVMTRKQIELVRSLSKAGNNGPWVTHWEEMAKKTAIRRLFKYLPVSIEIQRAVSMDEKEPLTIDPADSSVLTGEYSVIDNSEE

SEQ ID NO: 11 - protein, 276 aa, Salmonella enterica
MPKQPPIAKADLQKTQGARPPTAVKNNNDVISFINQPSMKEQLAAALPRHMTAERMIRIATTEIRKVPALGDCDTMSFVSAIVQCSQLGLEPGGALGHAYLLPFGNRNEKSGKKNVQLIIGYRGMIDLARRSGQIASLSARVVREGDDFSFEFGLEEKLVHRPGENEDAPVTHVYAVARLKDGGTQFEVMTRKQIELVRAQSKAGNNGPWVTHWEEMAKKTAIRRLFKYLPVSIEIQRAVSMDEKETLTIDPADASVITGEYSVVENAGVEENVTA

SEQ ID NO: 12 - protein, 266 aa, Mycobacterium tuberculosis
MSNPPIAQADLQKAQGTAVKEKTKDQQLIQFINQPGMKAQLSAALPRHITPDRMIRIVTTEIRKTPSLATCDMQSFIGAVVQCSQLGLEPGNALGHAYLLPFGNGKATSGQPNVQLIIGYRGMIDLARRSGQIISISARSVREGDSFHFEYGLNEDLTHVPGENDSGPITHVYAVARLKEGGVQFEVMSFSQIEKVRDSSKAGKNGPWVSHWEEMAKKTVIRRLFKYLPVSIEMQRAVILDEKAEANVDQEHASIFEGEYETVSPE

SEQ ID NO: 13 - protein, 338 aa, Bacillus anthracis
MSNDLTQITQRSLDEQVIGNLNRLQEQGLEMPPGYSPQNALKSAFFELTNNSGGNLLQLAANNPETKTSISNALLDMVIQGLSPAKKQCYFIKYGNKVQLMRSYFGTMAVLDRVTGGAEITPVVVREGDVFEIAMDGPDLVVAKHETSFENLDNDIKAAYVVIKLANGKEVTTVMTKKQIDKSWSKAKTKNVQNDFPEEMAKRTVINRAAKYLINTSNDNDLFVQAAKDTLENEFERKDVTPEREEQTAVLEEKIFTNNKKVIEQENDIERITRVADVPEQPDIEQAKQIEKEDLTKVADQILEEPVQETLDVMAGYETNQKESEADVSTIEEDDYPF

SEQ ID NO: 14 - protein, 268 aa, Clostridium tetani
MATNESLKNQLTTKKETGLGSAGNTIKGLMNSPAIKKRFEEVLKQRAPQYMSSIVNLVNSDINLKKCDQMSVVASCMVAATLDLPVDKNLGYAWVVPYGNKAQFQLGYKGYVQLALRTGQYKSINVIEIHEGELIDWNPLTEELKIDFSKKESDAVIGYAGYFELLNGFKKSTYWTKEQITKHKNKFSKSDFGWKKDFDAMARKTVLRNMLSKWGILSIEMQNAYTADQGIIKNEIMETGEVKENIEYIEADFESYEDNSIEEGGANE

SEQ ID NO: 15 - protein, 277 aa, Clostridium difficile
MASEKAKGALEKKVSGANTVKVSPSKGMEQLMNKMASQIKKALPSMVSSERFQRVALTAFSNNPRLQSCEPMSFIAAMMESAQLGLEPNTPLGQAYLIPYGNKVQFQIGYKGLLELAQRSGKIKTIYAHKIRENDKFEIKYGLHQDLVHEPKLNGDRGEIIGYYAVYHLDTGGHSFSFMTKEEIIEFAKSKSKSYSSGPWQTDFDSMAKKTVIKQLLKYAPLSIELQKAMVGDETIKSEIDEDMSMVVDESESLEVDFEVKENMDGKVSVEEAINVD

SEQ ID NO: 16 - protein, 303 aa, Haemophilus influenzae
MTNQVQHQQNKQPPALKTFFESANVQNKIKELVGKNAATFATSVMQIANSNSMLKTADPMSIFNAACMAATLNLPLQNGLGFAYIVPFRNNKEKKTEAQFQIGYKGFIQLAQRSGQFKRLVALPVYKKQLIKKDFINGFEFDWEQEPEQNENPIGYYAYFKLVNDFSAELYMSHDDIVKHAQRYSQTFKKGYGVWHDNFEAMALKTVTKLLLSKQAPLSVEMQQAVLADQTVVKDVENQEFNYTDNIQEAEFLAVVDEATFEQCKQSIANGETTLQELCDSGAYEFSQEQLTKLEELENQKAE

SEQ ID NO: 17 - protein, 306 aa, Staphylococcus aureus
MTENNKLQTIEQQLVQEKNVSDNVLNKVRVLESQGNLELPNDYSPSNAMKQAWLQISQDNKLMSCNDTSKANALLDMVTQGLNPAKNQCYFIPYGNKMQLQRSYHGNVMMLKRDAGAQDVVAQVIYKGDTFKQEMGGTGRIKAIKHEQDFFNIDKENIIGAYCTIVFNDGRDNYIEVMTIEQIKQAWMQSSMIKDEKALQNSKTHNNFKEEMAKKTVINRAAKRYINTSTDSNLFKYAQESEQRQRKEVLDAEVEENANQEQLDFEQPVLEEAQYTELENDKPIDVSDFEEIKEPATEKESEEEPF

SEQ ID NO: 18 - protein, 309 aa, Streptococcus pneumoniae
MANEIAKFDTLTPQQAFKSPAALEKFKSVLDGSETQFVASLLSIINNNSYLAQATNTSIMNAAMKAATLKLPIEPSLGMAYVVPYNRSEKRGNTWVKINEAQFQMGYKGFIQLAQRSGQIRNINCDVVYKEEFLRYDKVYGTLHLTDEQVDSGEVEGYFASLELINGFRKMIFWKKEKVIAHAQKYSKTYDKKTGDFKPGTPWKTEFDAMAQKTLIKELLSKYAPLSIELQKAILADNEDSNVNEVKRAKDVTPQEPENLSDLLSAPEEEQAKDVTPLEDDAQNSAADSVPDFVDPENGQMDMLEGEDF

SEQ ID NO: 19 - protein, 270 aa, Collinsella stercoris
MNQIVKFTDDSGLAVQVTPDDVRRYICENATEKEVGLFLQLCQTQRLNPFVKDAYLVKYGGAPASMITSYQVFNRRACRDANYDGIKSGVVVLRDGDVVHKRGAACYKKAGEELIGGWAEVRFKDGRETAYAEVALDDYSTGKSNWAKMPGVMIEKCAKAAAWRLAFPDTFQGMYAAEEMDQAQQPEQVRAQAEQPVDLQPIRELFKPYCEHFGITPAEGMTAVCGAVGAEGMHSMTEQQARRARAWMEEEMAAPAVEAEYEVVDEGEVF

SEQ ID NO: 20 - protein, 615 aa, Escherichia coli
MPIQVLPPQLANQIAAGEVVERPASVVKELVENSLDAGATRIDIDIERGGAKLIRIRDNGCGIKKDELALALARHATSKIASLDDLEAIISLGFRGEALASISSVSRLTLTSRTAEQQEAWQAYAEGRDMNVTVKPAAHPVGTTLEVLDLFYNTPARRKFLRTEKTEFNHIDEIIRRIALARFDVTINLSHNGKIVRQYRAVPEGGQKERRLGAICGTAFLEQALAIEWQHGDLTLRGWVADPNHTTPALAEIQYCYVNGRMMRDRLINHAIRQACEDKLGADQQPAFVLYLEIDPHQVDVNVHPAKHEVRFHQSRLVHDFIYQGVLSVLQQQLETPLPLDDEPQPAPRSIPENRVAAGRNHFAEPAAREPVAPRYTPAPASGSRPAAPWPNAQPGYQKQQGEVYRQLLQTPAPMQKLKAPEPQEPALAANSQSFGRVLTIVHSDCALLERDGNISLLSLPVAERWLRQAQLTPGEAPVCAQPLLIPLRLKVSAEEKSALEKAQSALAELGIDFQSDAQHVTIRAVPLPLRQQNLQILIPELIGYLAKQSVFEPGNIAQWIARNLMSEHAQWSMAQAITLLADVERLCPQLVKTPPGGLLQSVDLHPAIKALKDE

SEQ ID NO: 21 - DNA, 2311 bp, Escherichia coli
  source 1..2311: mol_type = other DNA
atgtagcgaccagtatgatcagtcagttgcaacgcattggcgaaatacataaacgccgaccagaacacgcgagtctcggcgttctgcgttcgccggatatcccgtcagtactggtcgaaaccggttttatcagcaacaacagcgaagaacgtttgctggcgagcgacgattaccaacaacagctggcagaagccatttacaaaggcctgcgcaattatttccttgcgcatccgatgcaatctgcgccgcagggggcaacggcacaaactgccagtacggtgacgacgccagatcgcacgctgccaaactaaggacgattgatgccaattcaggtcttaccgccacaactggcgaaccagattgccgcaggtgaggtggtcgagcgacctgcgtcggtagtcaaagaactggtggaaaacagcctcgatgcaggtgcgacacgtatcgatattgatatcgaacgcggtggggcgaaacttatccgcattcgtgataacggctgcggtatcaaaaaagacgagctggcgctggcgctggcgcgtcatgccaccagtaaaatcgcctctctggacgatctcgaagccattatcagcctgggctttcgcggtgaggcgctggcgagtatcagttcggtttcccgcctgacgctcacttcacgcaccgcagaacagcaggaagcctggcaggcctatgccgaagggcgcgatatgaacgtgacggtaaaaccggcggcgcatcctgtggggacgacgctggaggtgctggatctgttctacaacaccccggcgcggcgcaaattcctgcgcaccgagaaaaccgaatttaaccacattgatgagatcatccgccgcattgcgctggcgcgtttcgacgtcacgatcaacctgtcgcataacggtaaaattgtgcgtcagtaccgcgcagtgccggaaggcgggcaaaaagaacggcgcttaggcgcgatttgcggcaccgcttttcttgaacaagcgctggcgattgaatggcaacacggcgatctcacgctacgcggctgggtggccgatccaaatcacaccacgcccgcactggcagaaattcagtattgctacgtgaacggtcgcatgatgcgcgatcgcctgatcaatcacgcgatccgccaggcctgcgaagacaaactgggggccgatcagcaaccggcatttgtgctgtatctggagatcgatccgcatcaggtggacgtcaacgtgcaccccgccaaacacgaagtgcgtttccatcagtcgcgtctggtgcatgattttatctatcagggcgtgctgagcgtgctacaacagcaactggaaacgccgctaccgctggacgatgaaccccaacctgcaccgcgttccattccggaaaaccgcgtggcggcggggcgcaatcactttgcagaaccggcagctcgtgagccggtagctccgcgctacactcctgcgccagcatcaggcagtcgtccggctgccccctggccgaatgcgcagccaggctaccagaaacagcaaggtgaagtgtatcgccagcttttgcaaacgcccgcgccgatgcaaaaattaaaagcgccggaaccgcaggaacctgcacttgcggcgaacagtcagagttttggtcgggtactgactatcgtccattccgactgtgcgttgctggagcgcgacggcaacatttcacttttatccttgccagtggcagaacgttggctgcgtcaggcacaattgacgccgggtgaagcgcccgtttgcgcccagccgctgctgattccgttgcggctaaaagtttctgccgaagaaaaatcggcattagaaaaagcgcagtctgccctggcggaattgggtattgatttccagtcagatgcacagcatgtgaccatcagggcagtgcctttacccttacgccaacaaaatttacaaatcttgattcctgaactgataggctacctggcgaagcagtccgtattcgaacctggcaatattgcgcagtggattgcacgaaatctgatgagcgaacatgcgcagtggtcaatggcacaggccataaccctgctggcggacgtggaacggttatgtccgcaacttgtgaaaacgccgccgggtggtctgttacaatctgttgatttacatccggcgataaaagccctgaaagatgagtgatatcagtaaggcgagcctgcctaaggcgatttttttgatggggccgacggcctccggtaaaacggcgttagccattgagctgcgtaaaattttaccagtagagttgataagcgttgattctgcccttatttacaaagggatgg

SEQ ID NO: 22 - protein, 632 aa, Mycobacterium tuberculosis
MAIRILPPQLANQIAAGEVVERPASVVKELVENSLDAGATRIDIDIERGGEKLIRIRDNGCGIAKDELALALARHATSKIATLDDLEAIMSMGFRGEALASISSVSRLTFTSRTRDQNEAWQAYAEGREMAVTLKPAAHPAGSTVEVLDLFFNTPARRKFLRTEKTEFGHIDEVVRRIALSRFDVAINLTHNGKLMRQYRPVKAEDQQERRLGAICGTAFMQQALAVTWSHEELEIRGWVASPAEYNGPADLQYCYVNGRMMRDRLINHAIRQAYEDRLSGDQQPAYVLYLTIDPRQVDVNVHPAKHEVRFHQARLVHDFIYQAVLSVLKQHEQPVSLFQPDPSAPQTQHVPENRAAAGKNIFEREEALTPPPHRETTGGGASHSGHSSGKPAYSADKPVYSPKEAGIYQSLMQTPAETRPQLFPEKSTFLSENRISAASEPVTEKSERVSFGKILTLYPPCYALIETDAGIALFSLSKASHYLRCQQLIPGEQGLKSQPLMIPLQMALNAQECETFTQFAGVLRTFGIEGSVSRGKATIRTVSLPLRQQNLPHLIPELLRFLADNPEGDEKAIASRLAEMLVSEPAAQSKAQAVQLLADVERLCPQLVRRPPADLLQLIDLTEVVAALRHE

SEQ ID NO: 23 - protein, 618 aa, Salmonella enterica
MPIQVLPPQLANQIAAGEVVERPASVVKELVENSLDAGATRVDIDIERGGAKLIRIRDNGCGIKKEELALALARHATSKIASLDDLEAIISLGFRGEALASISSVSRLTLTSRTAEQAEAWQAYAEGRDMDVTVKPAAHPVGTTLEVLDLFYNTPARRKFMRTEKTEFNHIDEIIRRIALARFDVTLNLSHNGKLVRQYRAVAKDGQKERRLGAICGTPFLEQALAIEWQHGDLTLRGWVADPNHTTTALTEIQYCHVNGRMMRDRLINHAIRQACEDKLGADQQPAFVLYLEIDPHQVDVNVHPAKHEVRFHQSRLVHDFIYQGVLSVLQQQTETTLPLEDIAPAPRHVPENRIAAGRNHFAVPAEPTAAREPATPRYSGGASGGNGGRQSAGGWPHAQPGYQKQQGEVYRALLQTPTTSPAPEAVAPALDGHSQSFGRVLTIVCGDCALLEHAGTIQLLSLPVAERWLRQAQLTPGQSPVCAQPLLIPLRLKVSADEKAALQKAQSLLGELGIEFQSDAQHVTIRAVPLPLRQQNLQILIPELIGYLAQQTTFATVNIAQWIARNVQSEHPQWSMAQAISLLADVERLCPQLVKAPPGGLLQPVDLHSAMNALKHE

SEQ ID NO: 24 - protein, 626 aa, Bacillus anthracis
MGKIRKLDDQLSNLIAAGEVVERPASVVKELVENSIDANSTSIEIHLEEAGLSKIRIIDNGDGIAEEDCIVAFERHATSKIKDENDLFRIRTLGFRGEALPSIASVSELELITSTGDAPGTHLIIKGGDIIKQEKTASRKGTDITVQNLFFNTPARLKYMKTIHTELGNITDIVYRIAMSHPEVSLKLFHNEKKLLHTSGNGDVRQVLASIYSIQVAKKLVPIEAESLDFTIKGYVTLPEVTRASRNYMSTIVNGRYVRNFVLMKAIQQGYHTLLPVGRYPIGFLSIEMDPMLVDVNVHPAKLEVRFSKEQELLKLIEETLQAAFKKIQLIPDAGVTTKKKEKDESVQEQFQFEHAKPKEPSMPEIVLPTGMDEKQEEPQAVKQPTQLWQPSTKPIIEEPIQEEKSWDSNEEGFELEELEEVREIKEIEMNGNDLPPLYPIGQMHGTYIFAQNDKGLYMIDQHAAQERINYEYFRDKVGRVAQEVQELLVPYRIDLSLTEFLRVEEQLEELKKVGLFLEQFGHQSFIVRSHPTWFPKGQETEIIDEMMEQVVKLKKVDIKKLREEAAIMMSCKASIKANQYLTNDQIFALLEELRTTTNPYTCPHGRPILVHHSTYELEKMFKRVM

SEQ ID NO: 25 - protein, 620 aa, Clostridium tetani
MKRINILDECTFNKIAAGEVVERPFSVVKELVENSIDAEAKNITVEVKNGGQDLIKVSDDGAGIYADDIQKAFLTHATSKILNIDDIFSLNTMGFRGEALPSIASISKILLKSKPLSETSGKEIYMEGGNFISFNDVGMNTGTTIKVTDLFYNVPARLKFLKSSSRESSLISDIIQRLSLANPDIAFKLINNGKTVLNTYGSGNLEDAIRVIYGKKTLENISYFESHSDIISVYGYIGNAELSRGSRNNQSIFVNKRYIKSGLITAAVENAFKSFLTINKFPFFVIFIDIFPEYIDVNVHPTKTEIKFKEDKIVFSFVFKTVHESIKKSLYKEFNEQIKEDVKEDNKEIIKENPSLFQNVEKVQIPIDLKSASMDIERKSLVNSVLCNENNIVKDNINKNIYIDTKENLSENKLKNILKENTEDMVSKLPDMKIIGQFDNTYILAESVKNLYIIDQHAAHEKILFETYRDKIKKDEVKSQLLLQPIVLELDSEDFSYYVDNKELFYKTGFNIEVFGENTINIREVPFIMGKPDINNLFMDIINNIKAMGSGETIEVKYDSIAMLACKSAVKAHDKLSKEEMEALINDLRFAKDPFNCPHGRPTIIKITSLELEKKFKRIQ

SEQ ID NO: 26 - protein, 655 aa, Clostridium difficile
MKNIINILDDLTINKIAAGEVVERPSSVVKELIENSIDAGANKISIDIIDGGKSLIKITDNGIGIPSSEVEKSFLRHATSKIKKIDDLYDLYSLGFRGEALASISAVSKLEMTTKTKDEIIGTKIYVEGGKIISKEPIGSTNGTTIIIKDIFFNTPARQKFLKSTHAETINISDLINKLAIGNPNIQFKYTNNNKQMLNTPGDGKLVNTIRSIYGKEITENIIDVEFKCNHFKMNGYIGNNNIYRSNKNLQHIYINKRFVKSKIIIDAITESYKSIIPIGKHVVCFLNIEVDPSCIDVNIHPNKLEIKFEKEQEVYIELRDFLKVKLIHSNLIGKYATYSDKKTQPRIAINSREKSTDYKLRNNDLLESTHKNSNITKGKDEVIEVVTLSSEKPINEFQSVSEVLNASVEDDVKNINYLSEDSVNDNIQEEFQVDGIKNEGNYYLGDSIKDSEEEYSCSSKRKFSLYGYSVIGVVFNTYIILSKDDSMYLLDQHAAHERILYERYMEKFYRQDINMQILLDPVVIEVSNVDMLQIENNLELFMKFGFELEIFGNNHIMVRCVPTIFGVPETEKFILQIIDNIEEITSNYDLKGERFASMACRSAIKANDKIYDIEIKSLLEQLEKCENPFTCPHGRPIMVEISKTEIEKMFKRIM

SEQ ID NO: 27 - protein, 629 aa, Haemophilus influenzae
MPIRILSPQLANQIAAGEVVERPASVVKELVENSLDAGANKIQIDIENGGANLIRIRDNGCGIPKEELSLALARHATSKIADLDDLEAILSLGFRGEALASISSVSRLTLTSRTEEQTEAWQVYAQGRDMETTIKPASHPVGTTVEVANLFFNTPARRKFLRTDKTEFAHIDEVIRRIALTKFNTAFTLTHNGKIVRQYRPAFDLNQQLKRVAVICGDDFVKNALRIDWKHDDLHLSGWVATPNFSRTQNDLSYCYINGRMVRDKVISHAIRQAYAQYLPTDAYPAFVLFIDLNPHDVDVNVHPTKHEVRFHQQRLIHDFIYEGISYALNNQEQLNWHTEQSAVENHEENTVREPQPNYSIRPNRAAAGQNSFAPQYHEKPQQNQPHFSNTPVLPNHVSTGYRDYRSDAPSKTEQRLYAELLRTLPPTAQKDISNTAQQNISDTAKIISTEIIECSSHLRALSLIENRALLLQQNQDFFLLSLEKLQRLQWQLALQQIQIEQQPLLIPIVFRLTEAQFQAWQQYSDNFKKIGFEFIENQAQLRLTLNKVPNVLRTQNLQKCVMAMLTRDENSSPFLTALCAQLECKTFDALADALNLLSETERLLTQTNRTAFTQLLKPVNWQPLLDEI

SEQ ID NO: 28 - protein, 669 aa, Staphylococcus aureus
MGKIKELQTSLANKIAAGEVVERPSSVVKELLENAIDAGATEISIEVEESGVQSIRVVDNGSGIEAEDLGLVFHRHATSKLDQDEDLFHIRTLGFRGEALASISSVAKVTLKTCTDNANGNEIYVENGEILNHKPAKAKKGTDILVESLFYNTPARLKYIKSLYTELGKITDIVNRMAMSHPDIRIALISDGKTMLSTNGSGRTNEVMAEIYGMKVARDLVHISGDTSDYHIEGFVAKPEHSRSNKHYISIFINGRYIKNFMLNKAILEGYHTLLTIGRFPICYINIEMDPILVDVNVHPTKLEVRLSKEEQLYQLIVSKIQEAFKDRILIPKNNLDYVPKKNKVLHSFEQQKIEFEQRQNTENNQEKTFSSEESNSKPFMEENQNDEIVIKEDSYNPFVTKTSESLIADDESSGYNNTREKDEDYFKKQQEILQEMDQTFDSNDGTTVQNYENKASDDYYDVNDIKGTKSKDPKRRIPYMEIVGQVHGTYIIAQNEFGMYMIDQHAAQERIKYEYFRDKIGEVTNEVQDLLIPLTFHFSKDEQLVIDQYKNELQQVGIMLEHFGGHDYIVSSYPVWFPKDEVEEIIKDMIELILEEKKVDIKKLREDVAIMMSCKKSIKANHYLQKHEMSDLIDQLREAEDPFTCPHGRPIIINFSKYELEKLFKRVM

SEQ ID NO: 29 - protein, 649 aa, Streptococcus pneumoniae
MSHIIELPEMLANQIAAGEVIERPASVVKELVENAIDAGSSQIIIEIEEAGLKKVQITDNGHGIAHDEVELALRRHATSKIKNQADLFRIRTLGFRGEALPSIASVSVLTLLTAVDGASHGTKLVARGGEVEEVIPATSPVGTKVCVEDLFFNTPARLKYMKSQQAELSHIIDIVNRLGLAHPEISFSLISDGKEMTRTAGTGQLRQAIAGIYGLASAKKMIEIENSDLDFEISGFVSLPELTRANRNYISLFINGRYIKNFLLNRAILDGFGSKLMVGRFPLAVIHIHIDPYLADVNVHPTKQEVRISKEKELMTLVSEAIANSLKEQTLIPDALENLAKSTVRNREKVEQTILPLKENTLYYEKTEPSRPSQTEVADYQVELTDEGQDLTLFAKETLDRLTKPAKLHFAERKPANYDQLDHPELDLASIDKAYDKLEREEASSFPELEFFGQMHGTYLFAQGRDGLYIIDQHAAQERVKYEEYRESIGNVDQSQQQLLVPYIFEFPADDALRLKERMPLLEEVGVFLAEYGENQFILREHPIWMAEEEIESGIYEMCDMLLLTKEVSIKKYRAELAIMMSCKRSIKANHRIDDHSARQLLYQLSQCDNPYNCPHGRPVLVHFTKSDMEKMFRRIQENHTSLRELGKY

SEQ ID NO: 30 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 31 - DNA, 537 bp, Escherichia coli
  source 1..537: mol_type = other DNA
atggccagcagaggcgtaaacaaggttattctcgttggtaatctgggtcaggacccggaagtacgctacatgccaaatggtggcgcagttgccaacattacgctggctacttccgaatcctggcgtgataaagcgaccggcgagatgaaagaacagactgaatggcaccgcgttgtgctgttcggcaaactggcagaagtggcgagcgaatatctgcgtaaaggttctcaggtttatatcgaaggtcagctgcgtacccgtaaatggaccgatcaatccggtcaggatcgctacaccacagaagtcgtggtgaacgttggcggcaccatgcagatgctgggtggtcgtcagggtggtggcgctccggcaggtggcaatatcggtggtggtcagccgcagggcggttggggtcagcctcagcagccgcagggtggcaatcagttcagcggcggcgcgcagtctcgcccgcagcagtccgctccggcagcgccgtctaacgagccgccgatggactttgatgatgacattccgttctga

SEQ ID NO: 32 - protein, 144 aa, Escherichia coli
MWKRGENKVILMGRAGKDAEVRYTPNGTAIASLTLATEISYNDNEGKEQKETEWHDIVIFGKKAEAAGKYFKKGMMLYFVGRIRNNKWQGTDGKMRSNKEIVIDNNGEMQMLPGAGPRNTSAEGQGGIENHDEPPFPDMNDYPQ

SEQ ID NO: 33 - protein, 154 aa, Klebsiella pneumoniae
MINIKYMRCIMWKRGENKVILMGRSGKDAELRYTPNGTAIASLTLATEISYSDNEGKEQKETEWHDIVIFGKKAEAAGKYFKKGMMLYFVGRIRNNKWQGTDGKMRSNKEIVIDNNGEMQMLPGAGPRNTSAEGQGGIENHDEPPFPDMNDYPQ

SEQ ID NO: 34 - protein, 144 aa, Klebsiella pneumoniae
MWKRGENKVILMGRAGKDAEVRYTPNGTAIASLTLATEISYSDNEGKEQKETEWHDIVIFGKKAEAAGKYFKKGMMLYFVGRIRNNKWQGTDGKMRSNKEIVIDNNGEMQMLPGAGPRNTSAEGQGGFENHDEPPFPDMNDYPQ

SEQ ID NO: 35 - protein, 144 aa, Entero bacterales
MWKRGENKVILMGRAGKDAEVRYTPNGTAIASLTLATEISYSDNEGKEQKETEWHDIVIFGKKAEAAGKYFKKGMMLYFVGRIRNNKWQGTDGKMRSNKEIVIDNNGEMQMLPGAGPRNTSAEGLGGTENHDEPPFPDMNDYPQ

SEQ ID NO: 36 - protein, 144 aa, Entero bacterales
MWKRGENKVILMGRAGKDAEVRYTPNGTAIASLTLATEISYSDNEGKEQKETEWHDIVIFGKKAEAAGKYFKKGMMLYFVGRIRNNKWQGTDGKMRSNKEIVIDNNGEMQMLPGAGPRNTSAEGQGGIENHDEPPFPDMNDYPQ

SEQ ID NO: 37 - protein, 144 aa, Salmonella enterica
MWKRGENKVILMGRAGKDAEVRYTPNGTAIASLTLATEISYSDNEGKEQKETEWHDIVIFGKKAEAAGKYFKKGMMLYFVGRIRNNKWQGTDGKMRSNKEIVIDNNGEMQMLPGAGPRNTSTEGLGGTENHDEPPFPDMNDYPQ

SEQ ID NO: 38 - protein, 144 aa, Salmonella enterica
MWKRGENKVILMGRAGKDAEVRYTPNGTAIASLTLATEISYSDNEGKEQKETEWHDIVIFGKKAEAAGKYFKKGMMLYFVGRIRNNKWQGTDGKMRSNKEIVIDNNGEMQMLPGAGPRNTSAEGHGGIENHDEPPFPDMNDYPQ

SEQ ID NO: 39 - protein, 142 aa, Enterobacter hormaechei
KRGENKVILMGRAGKDAEVRYTPNRTAIASLTLATEISYSDNEGKEQKETEWHDIVIFGKKAEAAGKYFKKGMMLYFVGRIRNNKWQGTDGKMRSNKEIVIDNNGEMQMLPGAGPRNTSAEGQGGIENHDEPPFPDMNDYPQ

SEQ ID NO: 40 - protein, 136 aa, Citrobacter freundii
VILMGRSGKDAELRYTPNGTAIASLTLATEISYSDNEGKEQKETEWHDIVIFGKKAEAAGKYFKKGMMLYFVGRIRNNKWQGTDGKMRSNKEIVIDNNGEMQMLPGAGPRNTSAEGQGGIENHDEPPFPDMNDYPQ

SEQ ID NO: 41 - DNA, 169 bp, synthetic construct
  source 1..169: mol_type = other DNA
tgcgcacccttagcgagaggtttatcattaaggtcaacctctggatgttgtttcggcatcctgcattgaatctgagttactgtctgttttccttgttggaacggagagcatcgcctgatgctctccgagccaaccaggaaacccgttttttctgacgtaagggtgcgca

SEQ ID NO: 42 - DNA, 963 bp, synthetic construct
  source 1..963: mol_type = other DNA
atgaaatctgcagagtatctgaatacgttccgccttaggaatttgggcctccccgtgatgaacaatctccacgatatgagcaaggcgactcgaatatccgtggaaacgctgagactgctcatctatacagcagactttcggtacaggatctacacggtcgaaaagaaggggcctgagaaacgcatgcgaacaatttatcaacctagccgagagctcaaggcgttgcagggctgggttcttcgaaacatccttgacaaactctcatcatcaccctttagtattgggtttgaaaagcaccaaagcatccttaacaacgcgacgccacacataggtgccaatttcatattgaacatcgacttggaggatttttttccgagcctcacagccaataaagtgttcggtgtttttcacagtcttgggtacaatcgccttattagttccgttcttaccaagatttgttgttacaagaatctcttgccccagggagcacccagcagtccgaaattggcgaatttgatttgttccaagctcgattatcgaatacaagggtacgcgggcagccggggactcatctatacccgctacgcagacgatcttacgctgtctgcccaatcaatgaagaaggtcgtaaaggcgcgggatttcttgttttctatcatcccgtccgagggcttggtaattaattccaaaaagacttgtatctcaggaccacgatctcagcgaaaagtgacaggactcgtcatttctcaagaaaaagtcggtatagggagagagaagtataaggaaatccgcgcgaagatccaccacatattctgtggcaagagcagcgagatagaacacgtccgaggctggttgtccttcatactgagcgtggactcaaaaagccaccgccggttgatcacctatatttcaaaactggaaaagaaatatggaaagaacccactcaacaaagctaaaacatag

SEQ ID NO: 43 - DNA, 1761 bp, synthetic construct
  source 1..1761: mol_type = other DNA
atgacaaaaacttcaaagctggatgcgctgcgggcggctactagtagggaagatttggcgaagattctcgacataaagttggtgtttctgacaaacgtgttgtaccgcataggatccgacaaccagtatacgcaattcacaatacccaaaaagggtaaaggtgtccgcaccatcagcgcaccaacggaccgacttaaggatatacagaggaggatttgtgatcttcttagtgactgtagggatgaaatctttgcgattaggaagatctctaataattactcattcggcttcgaaagaggaaaatcaattatactcaatgcttacaagcatcgagggaagcaaattatattgaacatcgaccttaaggacttctttgagagctttaactttgggagagtccgggggtactttctctccaaccaggacttcttgttgaacccagttgtggcaacaacgttggcgaaggccgcctgctacaacgggactctgcctcaggggtccccatgttcccctattataagtaaccttatctgtaacattatggacatgcggctcgcaaagctcgccaagaagtacggctgcacttatagtcgatatgcggatgacattacgatcagcaccaataaaaataccttcccgttggagatggcgactgtgcagcctgaaggggttgtgctgggcaaagtgctcgtaaaggagattgaaaattcaggtttcgagattaacgattctaagactagattgacctacaaaacaagtaggcaagaagtcaccgggctgacggttaatcggattgtaaacattgatcggtgctactacaaaaagacgagggcgctggctcacgcattgtatcggacaggagaatataaggtcccagacgagaacggtgttctggtatctggagggcttgacaagttggagggtatgtttgggtttatcgaccaggtggataaattcaacaacattaaaaaaaagttgaataagcaacccgacagatatgttctgacaaatgccactttgcacggatttaagctcaaattgaacgccagggagaaagcctatagcaaattcatctactacaaattcttccacggtaatacttgtcccacgatcataacagagggtaagacggataggatttaccttaaagctgccctccatagcctcgagacaagttatcctgaactgtttcgggagaaaacagatagtaagaagaaggagataaatctgaatatttttaaaagcaatgagaagaccaagtatttcctggatctcagcggcggcacagcagacctcaagaaattcgtggaacgctacaaaaataactacgcttcctattacggcagcgtaccgaaacaaccggtgataatggtgcttgataacgacacaggcccgtcagacctgttgaactttttgagaaacaaagttaagagttgtccagatgatgtaacagaaatgcgcaagatgaagtacatacatgtgttttacaatctgtacatagttctgactcccctgtctccatctggagagcaaacgtctatggaggacctctttcctaaagatatattggacattaagatagatggcaagaaattcaataaaaacaatgacggtgactccaaaacagagtatgggaagcacatattctcaatgcgcgttgtacgagataaaaagaggaagatagatttcaaggcattttgctgtatcttcgatgctattaaggatattaaagaacattacaaactgatgttgaattcctag

SEQ ID NO: 44 - protein, 319 aa, synthetic construct
KSAEYLNTFRLRNLGLPVMNNLHDMSKATRISVETLRLLIYTADFRYRIYTVEKKGPEKRMRTIYQPSRELKALQGWVLRNILDKLSSSPFSIGFEKHQSILNNATPHIGANFILNIDLEDFFPSLTANKVFGVFHSLGYNRLISSVLTKICCYKNLLPQGAPSSPKLANLICSKLDYRIQGYAGSRGLIYTRYADDLTLSAQSMKKVVKARDFLFSIIPSEGLVINSKKTCISGPRSQRKVTGLVISQEKVGIGREKYKEIRAKIHHIFCGKSSEIEHVRGWLSFILSVDSKSHRRLITYISKLEKKYGKNPLNKAKT

SEQ ID NO: 45 - protein, 586 aa, synthetic construct
MTKTSKLDALRAATSREDLAKILDIKLVFLTNVLYRIGSDNQYTQFTIPKKGKGVRTISAPTDRLKDIQRRICDLLSDCRDEIFAIRKISNNYSFGFERGKSIILNAYKHRGKQIILNIDLKDFFESFNFGRVRGYFLSNQDFLLNPVVATTLAKAACYNGTLPQGSPCSPIISNLICNIMDMRLAKLAKKYGCTYSRYADDITISTNKNTFPLEMATVQPEGVVLGKVLVKEIENSGFEINDSKTRLTYKTSRQEVTGLTVNRIVNIDRCYYKKTRALAHALYRTGEYKVPDENGVLVSGGLDKLEGMFGFIDQVDKFNNIKKKLNKQPDRYVLTNATLHGFKLKLNAREKAYSKFIYYKFFHGNTCPTIITEGKTDRIYLKAALHSLETSYPELFREKTDSKKKEINLNIFKSNEKTKYFLDLSGGTADLKKFVERYKNNYASYYGSVPKQPVIMVLDNDTGPSDLLNFLRNKVKSCPDDVTEMRKMKYIHVFYNLYIVLTPLSPSGEQTSMEDLFPKDILDIKIDGKKFNKNNDGDSKTEYGKHIFSMRVVRDKKRKIDFKAFCCIFDAIKDIKEHYKLMLNS

SEQ ID NO: 46 - protein, 312 aa, synthetic construct
MSIDIETTLQKAYPDFDVLLKSRPATHYKVYKIPKRTIGYRIIAQPTPRVKAIQRDIIEILKQHTHIHDAATAYVDGKNILDNAKIHQSSVYLLKLDLVNFFNKITPELLFKALARQKVDISDTNKNLLKQFCFWNRTKRKNGALVLSVGAPSSPFISNIVMSSFDEEISSFCKENKISYSRYADDLTFSTNERDVLGLAHQKVKTTLIRFFGTRIIINNNKIVYSSKAHNRHVTGVTLTNNNKLSLGRERKRYITSLVFKFKEGKLSNVDINHLRGLIGFAYNIEPAFIERLEKKYGESTIKSIKKYSEGG

SEQ ID NO: 47 - protein, 311 aa, synthetic construct
MDILQHISDLLLTKKSEIISFSLTAPYRYKIYKIAKRNSDKKRTIAHPSKELKFIQREITEYLTDKLPVHECAFAYKKGSSIKTNAQVHLHTKYLLKMDFENFFPSITPRLFFSKLRLANIDLTADDKVLLENILFFKSKRNSNLRLSIGAPSSPLISNFVMYFWDIEVQEICSKIGVNYTRYADDLTFSTNNKDVLFDIPDMLENVLPKYSLGRIRINHEKTVFSSKGHNRHVTGITLTNDNKLSIGRERKRKISAMIHHFINGKLSTDECNKLVGLLAFAKNIEPSFYKSMVIKYGSDNIYKLQKQKDK

SEQ ID NO: 48 - DNA, 169 bp, synthetic construct; identical to SEQ ID NO: 41
  source 1..169: mol_type = other DNA

SEQ ID NO: 49 - DNA, 199 bp, synthetic construct
  source 1..199: mol_type = other DNA
tgataagattccgtatgcgcacccttagcgagaggtttatcattaaggtcaacctctggatgttgtttcggcatcctgcattgaatctgagttactgtctgttttccttgttggaacggagagcatcgcctgatgctctccgagccaaccaggaaacccgttttttctgacgtaagggtgcgcatacggaatcttatca